(19) United States
(12) Patent Application Publication
(10) Pub. No.: US 2024/0255524 A1
(43) Pub. Date: Aug. 1, 2024

(54) Renal Insufficiency Prediction and Uses Thereof

(71) Applicant: SomaLogic Operating Co., Inc., Boulder, CO (US)

(72) Inventors: Laura Sampson, Boulder, CO (US); Missy Simpson, Boulder, CO (US); Yolanda Hagar, Boulder, CO (US); Rachel Marie Ostroff, Westminster, CO (US)

(73) Assignee: SomaLogic Operating Co., Inc., Boulder, CO (US)

(21) Appl. No.: 18/566,223

(22) PCT Filed: Jun. 14, 2022
(86) PCT No.: PCT/US2022/033333

§ 371 Date: Dec. 1, 2023

Related U.S. Application Data
(60) Provisional application No. 63/210,600, filed on Jun. 15, 2021.

(51) Int. Cl. G01N 33/68

(52) U.S. Cl. CPC G01N 33/6887; G01N 33/6893; G01N 2333/78; G01N 2800/347

(57) ABSTRACT

The present disclosure includes biomarkers, methods, devices, reagents, systems, and kits for the evaluation of risk of renal insufficiency within a specified timeframe, for example 4 years. In one aspect, the disclosure provides biomarkers that can be used alone or in various combinations to evaluate risk of renal insufficiency within 4 years. In another aspect, methods are provided for evaluating risk of renal insufficiency within 4 years in an individual, where the methods include detecting, in a biological sample from an individual, at least one biomarker value corresponding to at least one biomarker selected from the group of biomarkers provided in Table 8.

### CROSS-REFERENCE TO RELATED APPLICATIONS

The present application claims the benefit of priority of U.S. Provisional Application No. 63/210,600, filed Jun. 15, 2021, which is incorporated by reference herein in its entirety for any purpose.

### FIELD OF THE INVENTION

The present application relates generally to the detection of biomarkers and a method of evaluating the risk of a future renal insufficiency in an individual and, more specifically, to one or more biomarkers, methods, devices, reagents, systems, and kits used to assess an individual for the prediction of risk of developing a renal insufficiency within a 4 year period.

### BACKGROUND

The following description provides a summary of information relevant to the present application and is not an admission that any of the information provided or publications referenced herein is prior art to the present application.

Chronic kidney disease (CKD) is defined as having abnormalities of kidney structure or function present for >3 months (Table 1) and affects approximately 13% of adults in the US; risk factors for the disease are heterogeneous and include genetic and demographic predisposition and diabetes. Kidneys serve three primary functions: they filter metabolic byproducts from the blood, produce urine and, in so doing, help to regulate blood pressure and fluid and electrolyte balance, and secrete hormones. Depending on the stage of kidney disease (FIG. 1), a range of symptoms and conditions can result including hypertension, peripheral vascular disease, atherosclerosis, chronic anemia, chronic fatigue, uremia, and cardiovascular disease. Chronic kidney disease is often asymptomatic in the earlier stages of disease (stages 1 and 2) and, because this is the time in the progression of disease when kidney function can still be preserved, identification of patients early in disease may help to slow or prevent progression of disease. Stages 3a to stage 4 are considered moderate to severe kidney disease, stage 5 is end stage renal disease. Progression through stages of kidney disease depends on the underlying cause of disease, presence of comorbid conditions, treatment, genetics, socioeconomic factors, and other factors.

TABLE 1
Criteria for CKD (either of the following present for >3 months)

| | |
|---|---|
| Markers of kidney | Albuminuria (albumin creatinine ratio ≥ 30 mg/g) |
| damage (one or | Urine sediment abnormalities |
| more) | Electrolyte and other abnormalities due to tubular |
| | disorders |
| | Abnormalities detected by histology |
| | Structural abnormalities detected by imaging |
| | History of kidney transplantation |
| Decreased GFR | GFR < 60 ml/min/1.73 m² |

Currently the standard of care for kidney disease prognosis is based on current clinical laboratory parameters (e.g., eGFR, albuminuria, packed cell volume) and comorbidities or by using the kidney failure risk equation (KFRE, Eq 1). (Tangri N, Stevens L A, Griffith J, et al. A predictive model for progression of chronic kidney disease to kidney failure. JAMA 2011; 305:1553-1559. doi: 1510.1001/jama.2011.1451. Epub 211 April 1511.) The KFRE was developed in patients with moderate to severe kidney disease (stage 3a-stage 4) and did not include earlier stages of kidney disease when there is more kidney function to preserve.

Using current clinical parameters as a prognostic tool is imprecise and may not identify all patients who would benefit from more aggressive medical treatment to prevent progression of disease. Recommendations for management of kidney disease include those shown in Table 2. (Chapter 2: Definition, identification, and prediction of CKD progression. 2011) 2013; 3:63-72. doi: 10.1038/kisup.2012.1065.)

TABLE 2
Recommendations for management of kidney disease

1. Prevention of CKD progression
1.1 Individualize blood pressure targets and agents according to age,
cardiovascular disease, and other comorbidities
1.1a Adults with CKD and urinary albumin < 30 mg/24 hours and
blood pressure consistently > 140/90 mm Hg be treated with BP-lowering
medications to maintain BP ≤ 140/90
1.1b Adults with CKD and urinary albumin ≥ 30 mg/24 hours and
blood pressure consistently > 130/80 mm Hg be treated with BP-lowering
medications to maintain BP ≤ 130-80
1.1b Adults with CKD and urinary albumin ≥ 30 mg/24 hours should
be on an ACE-inhibitor or angiotensin receptor blocker
1.2 Manage the risk for acute kidney injury
1.3 Decrease protein intake
1.4 Maintain glycemic control, target HbA1c~7.0%
1.5 Decrease salt intake
1.6 Engage in physical activity compatible with cardiovascular health and
tolerance
2. Manage complications associated with loss of kidney function
2.1 Diagnose anemia when haemoglobin concentration is <13.0 g/dl
2.2 It is not recommended that bone mineral density testing be performed
routinely
2.3 Monitor serum calcium, phosphate, PTH, and alkaline phosphate
levels
3. CKD and CVD
3.1 All people with CKD are considered at increased risk for CVD
3.2 Patients with CKD at risk for atherosclerotic events should be offered
treatment with antiplatelet agents unless there is an increased bleeding
risk.
3.3 Level of care for heart failure offered to people with CKD is the same
as is offered to those without CKD
3.4 People with CKD presenting for symptoms of acute cardiac injury
(chest pain, shortness of breath) should be investigated for underlying
cardiac disease
3.5 People with CKD shoud be regularly monitored for signs of peripheral
arterial disease and be considered for usual approaches to therapy.
3.6 in people with eGFR < 60 ml/min/1.73 m², NT-proBNP and Troponin
should be interpreted with caution
4. Medication management and patient safety
4.1 Take GFR into account when dosing drugs
4.2 Use Cystatin C or direct measurement of GFR when calculating
dosages for medications that require a high level of precision.

Chronic kidney disease may be prevented by aggressive treatment if the propensity for such disease can be accurately determined. Existing multi-marker tests either require the collection of multiple samples from an individual or require that a sample be partitioned between multiple assays. Optimally, an improved test would require only a single blood, urine or other sample, and a single assay. Accordingly, a need exists for biomarkers, methods, devices, reagents, systems, and kits that enable the prediction of the development of renal disease within a specified timeframe, such as a 4 year period.

### SUMMARY OF THE INVENTION

The present application includes biomarkers, methods, reagents, devices, systems, and kits for the prediction of risk of developing renal insufficiency within a specified timeframe, such as a 4 year period. In certain aspects, a kidney disease progression test is disclosed that predicts the development of at least one of the following within 4 years: a 50% decline in estimated glomerular filtration rate (eGFR), a diagnosis that kidney dialysis is needed, development of eGFR<15 ml/min/1.73 m², development of end stage renal disease (ESRD), or a diagnosis that a kidney transplantation is needed.

In one aspect, the kidney disease progression test disclosed herein is intended to provide a four year prognosis for Progressive Chronic Renal Insufficiency (PCRI) and includes patients who have earlier stages of kidney disease (stage 1-stage 2) compared to the population used to develop the KFRE, who are candidates for aggressive medical treatment to prevent disease progression (Table 2). In a further aspect, the presently disclosed test does not require the calculation of eGFR, measurement of proteinuria, or reliance on patient characteristics such as age or sex.

Benefits of the kidney disease progression tests disclosed herein include: convenience of a prognostic test for people with diagnosed chronic kidney disease that does not require estimating current kidney function (via eGFR), measurement of proteinuria, or input of age or sex; identification of patients at high risk for PCRI early in the disease process, identification of patients who may benefit from more aggressive medical management of kidney disease, and the metric (relative risk) delivered to patients provides a context to the reported value so that a person can understand their risk for severe kidney decline relative to an “average” or “typical” person with the same disease process as them.

The following numbered paragraphs [0011]-[00122] contain statements of broad combinations of the inventive technical features herein disclosed:
1. A method comprising:
a) measuring the level of COL28A1 protein, and the level of at least one, two, three, four, five, six, seven, eight, or nine proteins selected from the group consisting of HAVCR1, FSTL3, RGMB, UBE2G2, REG1A, REG1B, COL6A3, CST3, and TNFRSF1A in a sample from a human subject; and
b) identifying the human subject as being at relative risk for developing progressive chronic renal insufficiency within a 4 year period based on the level of COL28A1 and the level of the at least one, two, three, four, five, six, seven, eight, or nine proteins.
2. A method comprising:
a) measuring the level of UBE2G2 protein, and the level of at least one, two, three, four, five, six, seven, eight, or nine proteins selected from the group consisting of HAVCR1, FSTL3, RGMB, COL28A1, REG1A, REG1B, COL6A3, CST3, and TNFRSF1A in a sample from a human subject; and
b) identifying the human subject as being at relative risk for developing progressive chronic renal insufficiency within a 4 year period based on the level of UBE2G2 and the level of the at least one, two, three, four, five, six, seven, eight, or nine proteins.
3. A method comprising:
a) measuring the level of REG1B protein, and the level of at least one, two, three, four, five, six, seven, eight, or nine proteins selected from the group consisting of HAVCR1, FSTL3, RGMB, COL28A1, UBE2G2, REG1A, COL6A3, CST3, and TNFRSF1A in a sample from a human subject; and
b) identifying the human subject as being at relative risk for developing progressive chronic renal insufficiency within a 4 year period based on the level of REG1B and the level of the at least one, two, three, four, five, six, seven, eight, or nine proteins.
4. The method of aspect 1, wherein the method comprises measuring COL28A1 and HAVCR1, COL28A1 and FSTL3, COL28A1 and RGMB, COL28A1 and UBE2G2, COL28A1 and REG1A, COL28A1 and REG1B, COL28A1 and COL6A3, COL28A1 and CST3, or COL28A1 and TNFRSF1A.
5. The method of aspect 1, wherein the method comprises measuring COL28A1, HAVCR1, and FSTL3; COL28A1, HAVCR1, and RGMB; COL28A1, HAVCR1, and UBE2G2; COL28A1, HAVCR1, and REG1A; COL28A1, HAVCR1, and REG1B; COL28A1, HAVCR1, and COL6A3; COL28A1, HAVCR1, and CST3; COL28A1, HAVCR1, and TNFRSF1A; COL28A1, FSTL3, and RGMB; COL28A1, FSTL3, and UBE2G2; COL28A1, FSTL3, and REG1A; COL28A1, FSTL3, and REG1B; COL28A1, FSTL3, and COL6A3; COL28A1, FSTL3, and CST3; COL28A1, FSTL3, and TNFRSF1A; COL28A1, RGMB, and UBE2G2; COL28A1, RGMB, and REG1A; COL28A1, RGMB, and REG1B; COL28A1, RGMB, and COL6A3; COL28A1, RGMB, and CST3; COL28A1, RGMB, and TNFRSF1A; COL28A1, UBE2G2, and REG1A; COL28A1, UBE2G2, and REG1B; COL28A1, UBE2G2, and COL6A3; COL28A1, UBE2G2, and CST3; COL28A1, UBE2G2, and TNFRSF1A; COL28A1, REG1A, and REG1B; COL28A1, REG1A, and COL6A3; COL28A1, REG1A, and CST3; COL28A1, REG1A, and TNFRSF1A; COL28A1, REG1B, and COL6A3; COL28A1, REG1B, and CST3; COL28A1, REG1B, and TNFRSF1A; COL28A1, COL6A3, and CST3; COL28A1, COL6A3, and TNFRSF1A; or COL28A1, CST3, and TNFRSF1A.
6. The method of aspect 2, wherein the method comprises measuring UBE2G2 and HAVCR1, UBE2G2 and FSTL3, UBE2G2 and RGMB, UBE2G2 and COL28A1, UBE2G2 and REG1A, UBE2G2 and REG1B, UBE2G2 and COL6A3, UBE2G2 and CST3, or UBE2G2 and TNFRSF1A.
7. The method of aspect 2, wherein the method comprises measuring UBE2G2, HAVCR1, and FSTL3; UBE2G2, HAVCR1, and RGMB; UBE2G2, HAVCR1, and COL28A1; UBE2G2, HAVCR1, and REG1A; UBE2G2, HAVCR1, and REG1B; UBE2G2, HAVCR1, and COL6A3; UBE2G2, HAVCR1, and CST3; UBE2G2, HAVCR1, and TNFRSF1A; UBE2G2, FSTL3, and RGMB; UBE2G2, FSTL3, and COL28A1; UBE2G2, FSTL3, and REG1A; UBE2G2, FSTL3, and REG1B; UBE2G2, FSTL3, and COL6A3; UBE2G2, FSTL3, and CST3; UBE2G2, FSTL3, and TNFRSF1A; UBE2G2, RGMB, and COL28A1; UBE2G2, RGMB, and REG1A; UBE2G2, RGMB, and REG1B; UBE2G2, RGMB, and COL6A3; UBE2G2, RGMB, and CST3; UBE2G2, RGMB, and TNFRSF1A; UBE2G2, COL28A1, and REG1A; UBE2G2, COL28A1, and REG1B; UBE2G2, COL28A1, and COL6A3; UBE2G2, COL28A1, and CST3; UBE2G2, COL28A1, and TNFRSF1A; UBE2G2, REG1A, and REG1B; UBE2G2, REG1A, and COL6A3; UBE2G2, REG1A, and CST3; UBE2G2, REG1A, and TNFRSF1A; UBE2G2, REG1B, and COL6A3; UBE2G2, REG1B, and CST3; UBE2G2, REG1B, and TNFRSF1A; UBE2G2, COL6A3, and CST3; UBE2G2, COL6A3, and TNFRSF1A; or UBE2G2, CST3, and TNFRSF1A.
8. The method of aspect 3, wherein the method comprises measuring REG1B and HAVCR1, REG1B and FSTL3, REG1B and RGMB, REG1B and COL28A1, REG1B and UBE2G2, REG1B and REG1A, REG1B and COL6A3, REG1B and CST3, or REG1B and TNFRSF1A.
9. The method of aspect 3, wherein the method comprises measuring REG1B, HAVCR1, and FSTL3; REG1B, HAVCR1, and RGMB; REG1B, HAVCR1, and COL28A1; REG1B, HAVCR1, and UBE2G2; REG1B, HAVCR1, and REG1A; REG1B, HAVCR1, and COL6A3; REG1B, HAVCR1, and CST3; REG1B, HAVCR1, and TNFRSF1A; REG1B, FSTL3, and RGMB; REG1B, FSTL3, and COL28A1; REG1B, FSTL3, and UBE2G2; REG1B, FSTL3, and REG1A; REG1B, FSTL3, and COL6A3; REG1B, FSTL3, and CST3; REG1B, FSTL3, and TNFRSF1A; REG1B, RGMB, and COL28A1; REG1B, RGMB, and UBE2G2; REG1B, RGMB, and REG1A; REG1B, RGMB, and COL6A3; REG1B, RGMB, and CST3; REG1B, RGMB, and TNFRSF1A; REG1B, COL28A1, and UBE2G2; REG1B, COL28A1, and REG1A; REG1B, COL28A1, and COL6A3; REG1B, COL28A1, and CST3; REG1B, COL28A1, and TNFRSF1A; REG1B, UBE2G2, and REG1A; REG1B, UBE2G2, and COL6A3; REG1B, UBE2G2, and CST3; REG1B, UBE2G2, and TNFRSF1A; REG1B, REG1A, and COL6A3; REG1B, REG1A, and CST3; REG1B, REG1A, and TNFRSF1A; REG1B, COL6A3, and CST3; REG1B, COL6A3, and TNFRSF1A; or REG1B, CST3, and TNFRSF1A.
10. The method of aspect 1, wherein the method comprises measuring COL28A1 and UBE2G2, and at least one of the following proteins selected from HAVCR1, FSTL3, RGMB, REG1A, REG1B, COL6A3, CST3, and TNFRSF1A.
11. The method of aspect 1, wherein the method comprises measuring COL28A1 and REG1B, and at least one of the following proteins selected from HAVCR1, FSTL3, RGMB, UBE2G2, REG1A, COL6A3, CST3, and TNFRSF1A.
12. The method of aspect 2, wherein the method comprises measuring UBE2G2 and REG1B, and at least one of the following proteins selected from HAVCR1, FSTL3, RGMB, COL28A1, REG1A, COL6A3, CST3, and TNFRSF1A.
13. The method of any one of aspects 1 to 12, wherein progressive chronic renal insufficiency within a 4 year period indicates the development of one or more of a 50% decline in estimated glomerular filtration rate (eGFR), a diagnosis that kidney dialysis is needed, development of eGFR<15 ml/min/1.73 m2, development of end stage renal disease (ESRD), or a diagnosis that a kidney transplantation is needed.
14. The method of any one of aspects 1 to 13, wherein the measuring is performed using mass spectrometry, an aptamer based assay and/or an antibody based assay.
15. The method of any one of aspects 1 to 14, wherein the sample is selected from blood, plasma, serum or urine.
16. A method comprising:
a) contacting a sample from a human subject with a set of capture reagents, wherein each capture reagent has affinity for a different protein of the set of proteins comprising COL28A1 protein, and at least one, two, three, four, five, six, seven, eight, or nine proteins selected from the group consisting of HAVCR1, FSTL3, RGMB, UBE2G2, REG1A, REG1B, COL6A3, CST3, and TNFRSF1A; and
b) measuring the level of each protein of the set of proteins with the set of capture reagents.
17. A method comprising:
a) contacting a sample from a human subject with a set of capture reagents, wherein each capture reagent has affinity for a different protein of the set of proteins comprising UBE2G2 protein, and at least one, two, three, four, five, six, seven, eight, or nine proteins selected from the group consisting of HAVCR1, FSTL3, RGMB, COL28A1, REG1A, REG1B, COL6A3, CST3, and TNFRSF1A; and
b) measuring the level of each protein of the set of proteins with the set of capture reagents.
18. A method comprising:
a) contacting a sample from a human subject with a set of capture reagents, wherein each capture reagent has affinity for a different protein of the set of proteins comprising REG1B protein, and at least one, two, three, four, five, six, seven, eight, or nine proteins selected from the group consisting of HAVCR1, FSTL3, RGMB, COL28A1, UBE2G2, REG1A, COL6A3, CST3, and TNFRSF1A; and
b) measuring the level of each protein of the set of proteins with the set of capture reagents.
19. The method of aspect 16, wherein the method comprises measuring COL28A1 and HAVCR1, COL28A1 and FSTL3, COL28A1 and RGMB, COL28A1 and UBE2G2, COL28A1 and REG1A, COL28A1 and REG1B, COL28A1 and COL6A3, COL28A1 and CST3, or COL28A1 and TNFRSF1A.
20. The method of aspect 16, wherein the method comprises measuring COL28A1, HAVCR1, and FSTL3; COL28A1, HAVCR1, and RGMB; COL28A1, HAVCR1, and UBE2G2; COL28A1, HAVCR1, and REG1A; COL28A1, HAVCR1, and REG1B; COL28A1, HAVCR1, and COL6A3; COL28A1, HAVCR1, and CST3; COL28A1, HAVCR1, and TNFRSF1A; COL28A1, FSTL3, and RGMB; COL28A1, FSTL3, and UBE2G2; COL28A1, FSTL3, and REG1A; COL28A1, FSTL3, and REG1B; COL28A1, FSTL3, and COL6A3; COL28A1, FSTL3, and CST3; COL28A1, FSTL3, and TNFRSF1A; COL28A1, RGMB, and UBE2G2; COL28A1, RGMB, and REG1A; COL28A1, RGMB, and REG1B; COL28A1, RGMB, and COL6A3; COL28A1, RGMB, and CST3; COL28A1, RGMB, and TNFRSF1A; COL28A1, UBE2G2, and REG1A; COL28A1, UBE2G2, and REG1B; COL28A1, UBE2G2, and COL6A3; COL28A1, UBE2G2, and CST3; COL28A1, UBE2G2, and TNFRSF1A; COL28A1, REG1A, and REG1B; COL28A1, REG1A, and COL6A3; COL28A1, REG1A, and CST3; COL28A1, REG1A, and TNFRSF1A; COL28A1, REG1B, and COL6A3; COL28A1, REG1B, and CST3; COL28A1, REG1B, and TNFRSF1A; COL28A1, COL6A3, and CST3; COL28A1, COL6A3, and TNFRSF1A; or COL28A1, CST3, and TNFRSF1A.
21. The method of aspect 17, wherein the method comprises measuring UBE2G2 and HAVCR1, UBE2G2 and FSTL3, UBE2G2 and RGMB, UBE2G2 and COL28A1, UBE2G2 and REG1A, UBE2G2 and REG1B, UBE2G2 and COL6A3, UBE2G2 and CST3, or UBE2G2 and TNFRSF1A.
22. The method of aspect 17, wherein the method comprises measuring UBE2G2, HAVCR1, and FSTL3; UBE2G2, HAVCR1, and RGMB; UBE2G2, HAVCR1, and COL28A1; UBE2G2, HAVCR1, and REG1A; UBE2G2, HAVCR1, and REG1B; UBE2G2, HAVCR1, and COL6A3; UBE2G2, HAVCR1, and CST3; UBE2G2, HAVCR1, and TNFRSF1A; UBE2G2, FSTL3, and RGMB; UBE2G2, FSTL3, and COL28A1; UBE2G2, FSTL3, and REG1A; UBE2G2, FSTL3, and REG1B; UBE2G2, FSTL3, and COL6A3; UBE2G2, FSTL3, and CST3; UBE2G2, FSTL3, and TNFRSF1A; UBE2G2, RGMB, and COL28A1; UBE2G2, RGMB, and REG1A; UBE2G2, RGMB, and REG1B; UBE2G2, RGMB, and COL6A3; UBE2G2, RGMB, and CST3; UBE2G2, RGMB, and TNFRSF1A; UBE2G2, COL28A1, and REG1A; UBE2G2, COL28A1, and REG1B; UBE2G2, COL28A1, and COL6A3; UBE2G2, COL28A1, and CST3; UBE2G2, COL28A1, and TNFRSF1A; UBE2G2, REG1A, and REG1B; UBE2G2, REG1A, and COL6A3; UBE2G2, REG1A, and CST3; UBE2G2, REG1A, and TNFRSF1A; UBE2G2, REG1B, and COL6A3; UBE2G2, REG1B, and CST3; UBE2G2, REG1B, and TNFRSF1A; UBE2G2, COL6A3, and CST3; UBE2G2, COL6A3, and TNFRSF1A; or UBE2G2, CST3, and TNFRSF1A.
23. The method of aspect 18, wherein the method comprises measuring REG1B and HAVCR1, REG1B and FSTL3, REG1B and RGMB, REG1B and COL28A1, REG1B and UBE2G2, REG1B and REG1A, REG1B and COL6A3, REG1B and CST3, or REG1B and TNFRSF1A.
24. The method of aspect 18, wherein the method comprises measuring REG1B, HAVCR1, and FSTL3; REG1B, HAVCR1, and RGMB; REG1B, HAVCR1, and COL28A1; REG1B, HAVCR1, and UBE2G2; REG1B, HAVCR1, and REG1A; REG1B, HAVCR1, and COL6A3; REG1B, HAVCR1, and CST3; REG1B, HAVCR1, and TNFRSF1A; REG1B, FSTL3, and RGMB; REG1B, FSTL3, and COL28A1; REG1B, FSTL3, and UBE2G2; REG1B, FSTL3, and REG1A; REG1B, FSTL3, and COL6A3; REG1B, FSTL3, and CST3; REG1B, FSTL3, and TNFRSF1A; REG1B, RGMB, and COL28A1; REG1B, RGMB, and UBE2G2; REG1B, RGMB, and REG1A; REG1B, RGMB, and COL6A3; REG1B, RGMB, and CST3; REG1B, RGMB, and TNFRSF1A; REG1B, COL28A1, and UBE2G2; REG1B, COL28A1, and REG1A; REG1B, COL28A1, and COL6A3; REG1B, COL28A1, and CST3; REG1B, COL28A1, and TNFRSF1A; REG1B, UBE2G2, and REG1A; REG1B, UBE2G2, and COL6A3; REG1B, UBE2G2, and CST3; REG1B, UBE2G2, and TNFRSF1A; REG1B, REG1A, and COL6A3; REG1B, REG1A, and CST3; REG1B, REG1A, and TNFRSF1A; REG1B, COL6A3, and CST3; REG1B, COL6A3, and TNFRSF1A; or REG1B, CST3, and TNFRSF1A.
25. The method of aspect 16, wherein the method comprises measuring COL28A1 and UBE2G2, and at least one of the following proteins selected from HAVCR1, FSTL3, RGMB, REG1A, REG1B, COL6A3, CST3, and TNFRSF1A.
26. The method of aspect 16, wherein the method comprises measuring COL28A1 and REG1B, and at least one of the following proteins selected from HAVCR1, FSTL3, RGMB, UBE2G2, REG1A, COL6A3, CST3, and TNFRSF1A.
27. The method of aspect 17, wherein the method comprises measuring UBE2G2 and REG1B, and at least one of the following proteins selected from HAVCR1, FSTL3, RGMB, COL28A1, REG1A, COL6A3, CST3, and TNFRSF1A.
28. The method of any one of aspects 16 to 27, wherein the protein levels are used to identify a human subject as being at relative risk for developing progressive chronic renal insufficiency within a 4 year period.
29. The method of aspect 28, wherein progressive chronic renal insufficiency within a 4 year period indicates the development of one or more of a 50% decline in estimated glomerular filtration rate (eGFR), a diagnosis that kidney dialysis is needed, development of eGFR<15 ml/min/1.73 m2, development of end stage renal disease (ESRD), or a diagnosis that a kidney transplantation is needed.
30. The method of any one of aspects 16 to 29, wherein the set of capture reagents is selected from aptamers, antibodies and a combinations of aptamers and antibodies.
31. The method of any one of aspects 16 to 30, wherein the sample is selected from blood, plasma, serum or urine.
32. A method comprising:
a) contacting a sample from a human subject with two capture reagents, wherein one capture reagent has affinity for a COL28A1 protein and the second capture reagent has affinity for a UBE2G2 protein; and
b) measuring the level of each protein with the two capture reagents.
33. A method comprising:
a) contacting a sample from a human subject with two capture reagents, wherein one capture reagent has affinity for a COL28A1 protein and the second capture reagent has affinity for a REG1B protein; and
b) measuring the level of each protein with the two capture reagents.
34. A method comprising:
a) contacting a sample from a human subject with two capture reagents, wherein one capture reagent has affinity for a UBE2G2 protein and the second capture reagent has affinity for a REG1B protein; and
b) measuring the level of each protein with the two capture reagents.
35. The method of any one of aspects 32 to 34, further comprising measuring the level of a HAVCR1 protein with a capture reagent having affinity for the HAVCR1 protein.
36. The method of any one of aspects 32 to 35, further comprising measuring the level of a FSTL3 protein with a capture reagent having affinity for the FSTL3 protein.
37. The method of any one of aspects 32 to 36, further comprising measuring the level of a RGMB protein with a capture reagent having affinity for the RGMB protein.
38. The method of any one of aspects 32 to 37, further comprising measuring the level of a REG1A protein with a capture reagent having affinity for the REG1A protein.
39. The method of any one of aspects 32 to 38, further comprising measuring the level of a COL6A3 protein with a capture reagent having affinity for the COL6A3 protein.
40. The method of any one of aspects 32 to 39, further comprising measuring the level of a CST3 protein with a capture reagent having affinity for the CST3 protein.
41. The method of any one of aspects 32 to 40, further comprising measuring the level of a TNFRSF1A protein with a capture reagent having affinity for the TNFRSF1A protein.
42. A method comprising:
a) measuring the level of COL28A1 and UBE2G2 in a sample from a human subject; and
b) identifying the human subject as being at relative risk for developing progressive chronic renal insufficiency within a 4 year period based on the level of COL28A1 and UBE2G2.
43. A method comprising:
a) measuring the level of COL28A1 and REG1B in a sample from a human subject; and
b) identifying the human subject as being at relative risk for developing progressive chronic renal insufficiency within a 4 year period based on the level of COL28A1 and REG1B.
44. A method comprising:
a) measuring the level of UBE2G2 and REG1B in a sample from a human subject; and
b) identifying the human subject as being at relative risk for developing progressive chronic renal insufficiency within a 4 year period based on the level of UBE2G2 and REG1B.
45. The method of any one of aspects 42 to 44, further comprising measuring the level of a HAVCR1 protein.
46. The method of any one of aspects 42 to 45, further comprising measuring the level of a FSTL3 protein.
47. The method of any one of aspects 42 to 46, further comprising measuring the level of a RGMB protein.
48. The method of any one of aspects 42 to 47, further comprising measuring the level of a REG1A protein.
49. The method of any one of aspects 42 to 48, further comprising measuring the level of a COL6A3 protein.
50. The method of any one of aspects 42 to 49, further comprising measuring the level of a CST3 protein.
51. The method of any one of aspects 42 to 50, further comprising measuring the level of a TNFRSF1A protein.
52. The method of any one of aspects 42 to 51, wherein the measuring is performed using mass spectrometry, an aptamer based assay and/or an antibody based assay.
53. A method comprising:
a) contacting a sample from a human subject with three capture reagents, wherein each of the three capture reagents has affinity for a protein selected from COL28A1, UBE2G2 and REG1B; and
b) measuring the level of each protein with the three capture reagents.
54. The method of aspect 53, further comprising measuring the level of a HAVCR1 protein with a capture reagent having affinity for the HAVCR1 protein.
55. The method of aspect 53 or 54, further comprising measuring the level of a FSTL3 protein with a capture reagent having affinity for the FSTL3 protein.
56. The method of any one of aspects 53 to 55, further comprising measuring the level of a RGMB protein with a capture reagent having affinity for the RGMB protein.
57. The method of any one of aspects 53 to 56, further comprising measuring the level of a REG1A protein with a capture reagent having affinity for the REG1A protein.
58. The method of any one of aspects 53 to 57, further comprising measuring the level of a COL6A3 protein with a capture reagent having affinity for the COL6A3 protein.
59. The method of any one of aspects 53 to 58, further comprising measuring the level of a CST3 protein with a capture reagent having affinity for the CST3 protein.
60. The method of any one of aspects 53 to 59, further comprising measuring the level of a TNFRSF1A protein with a capture reagent having affinity for the TNFRSF1A protein.
61. A method comprising:
a) measuring the level of COL28A1, UBE2G2 and REG1B in a sample from a human subject; and
b) identifying the human subject as being at relative risk for developing progressive chronic renal insufficiency within a 4 year period based on the level of COL28A1, UBE2G2 and REG1B.
62. The method of aspect 61, further comprising measuring the level of a HAVCR1 protein.
63. The method of aspect 61 or 62, further comprising measuring the level of a FSTL3 protein.
64. The method of any one of aspects 61 to 63, further comprising measuring the level of a RGMB protein.
65. The method of any one of aspects 61 to 64, further comprising measuring the level of a REG1A protein.
66. The method of any one of aspects 61 to 65, further comprising measuring the level of a COL6A3 protein.
67. The method of any one of aspects 61 to 66, further comprising measuring the level of a CST3 protein.
68. The method of any one of aspects 61 to 67, further comprising measuring the level of a TNFRSF1A protein.
69. The method of any one of aspects 61 to 68, wherein the measuring is performed using mass spectrometry, an aptamer based assay and/or an antibody based assay.
70. A method comprising:
a) measuring the level of at least three, four, five, six, seven, eight, nine or ten proteins selected from the group consisting of HAVCR1, FSTL3, RGMB, COL28A1, UBE2G2, REG1A, REG1B, COL6A3, CST3, and TNFRSF1A in a sample from a human subject; and
b) identifying the human subject as being at relative risk for developing progressive chronic renal insufficiency within a 4 year period based on the level of the at least three, four, five, six, seven, eight, nine or ten proteins.
71. The method of aspect 70, wherein the measuring is performed using mass spectrometry, an aptamer based assay and/or an antibody based assay.
72. The method of aspect 70 or 71, wherein the sample is selected from blood, plasma, serum or urine.
73. The method of any one of aspects 70 to 72, wherein the method comprises measuring HAVCR1, FSTL3, and RGMB; HAVCR1, FSTL3, and COL28A1; HAVCR1, FSTL3, and UBE2G2; HAVCR1, FSTL3, and REG1A; HAVCR1, FSTL3, and REG1B; HAVCR1, FSTL3, and COL6A3; HAVCR1, FSTL3, and CST3; HAVCR1, FSTL3, and TNFRSF1; HAVCR1, RGMB, and COL28A1; HAVCR1, RGMB, and UBE2G2; HAVCR1, RGMB, and REG1A; HAVCR1, RGMB, and REG1B; HAVCR1, RGMB, and COL6A3; HAVCR1, RGMB, and CST3; HAVCR1, RGMB, and TNFRSF1A; HAVCR1, COL28A1, and UBE2G2; HAVCR1, COL28A1, and REG1A; HAVCR1, COL28A1, and REG1B; HAVCR1, COL28A1, and COL6A3; HAVCR1, COL28A1, and CST3; HAVCR1, COL28A1, and TNFRSF1A; HAVCR1, UBE2G2, and REG1A; HAVCR1, UBE2G2, and REG1B; HAVCR1, UBE2G2, and COL6A3; HAVCR1, UBE2G2, and CST3; HAVCR1, UBE2G2, and TNFRSF1A; HAVCR1, REG1A, and REG1B; HAVCR1, REG1A, and COL6A3; HAVCR1, REG1A, and CST3; HAVCR1, REG1A, and TNFRSF1A; HAVCR1, REG1B, and COL6A3; HAVCR1, REG1B, and CST3; HAVCR1, REG1B, and TNFRSF1A; HAVCR1, COL6A3, and CST3; HAVCR1, COL6A3, and TNFRSF1A; HAVCR1, CST3, and TNFRSF1A; FSTL3, RGMB, and COL28A1; FSTL3, RGMB, and UBE2G2; FSTL3, RGMB, and REG1A; FSTL3, RGMB, and REG1B; FSTL3, RGMB, and COL6A3; FSTL3, RGMB, and CST3; FSTL3, RGMB, and TNFRSF1A; FSTL3, COL28A1, and UBE2G2; FSTL3, COL28A1, and REG1A; FSTL3, COL28A1, and REG1B; FSTL3, COL28A1, and COL6A3; FSTL3, COL28A1, and CST3; FSTL3, COL28A1, and TNFRSF1A; FSTL3, UBE2G2, and REG1A; FSTL3, UBE2G2, and REG1B; FSTL3, UBE2G2, and COL6A3; FSTL3, UBE2G2, and CST3; FSTL3, UBE2G2, and TNFRSF1A; FSTL3, REG1A, and REG1B; FSTL3, REG1A, and COL6A3; FSTL3, REG1A, and CST3; FSTL3, REG1A, and TNFRSF1A; FSTL3, REG1B, and COL6A3; FSTL3, REG1B, and CST3; FSTL3, REG1B, and TNFRSF1A; FSTL3, COL6A3, and CST3; FSTL3, COL6A3, and TNFRSF1A; FSTL3, CST3, and TNFRSF1A; RGMB, COL28A1, and UBE2G2; RGMB, COL28A1, and REG1A; RGMB, COL28A1, and REG1B; RGMB, COL28A1, and COL6A3; RGMB, COL28A1, and CST3; RGMB, COL28A1, and TNFRSF1A; RGMB, UBE2G2, and REG1A; RGMB, UBE2G2, and REG1B; RGMB, UBE2G2, and COL6A3; RGMB, UBE2G2, and CST3; RGMB, UBE2G2, and TNFRSF1A; RGMB, REG1A, and REG1B; RGMB, REG1A, and COL6A3; RGMB, REG1A, and CST3; RGMB, REG1A, and TNFRSF1A; RGMB, REG1B, and COL6A3; RGMB, REG1B, and CST3; RGMB, REG1B, and TNFRSF1A; RGMB, COL6A3, and CST3; RGMB, COL6A3, and TNFRSF1A; RGMB, CST3, and TNFRSF1A; COL28A1, UBE2G2, and REG1A; COL28A1, UBE2G2, and REG1B; COL28A1, UBE2G2, and COL6A3; COL28A1, UBE2G2, and CST3; COL28A1, UBE2G2, and TNFRSF1A; COL28A1, REG1A, and REG1B; COL28A1, REG1A, and COL6A3; COL28A1, REG1A, and CST3; COL28A1, REG1A, and TNFRSF1A; COL28A1, REG1B, and COL6A3; COL28A1, REG1B, and CST3; COL28A1, REG1B, and TNFRSF1A; COL28A1, COL6A3, and CST3; COL28A1, COL6A3, and TNFRSF1A; COL28A1, CST3, and TNFRSF1A; UBE2G2, REG1A, and REG1B; UBE2G2, REG1A, and COL6A3; UBE2G2, REG1A, and CST3; UBE2G2, REG1A, and TNFRSF1A; UBE2G2, REG1B, and COL6A3; UBE2G2, REG1B, and CST3; UBE2G2, REG1B, and TNFRSF1A; UBE2G2, COL6A3, and CST3; UBE2G2, COL6A3, and TNFRSF1A; UBE2G2, CST3, and TNFRSF1A; REG1A, REG1B, and COL6A3; REG1A, REG1B, and CST3; REG1A, REG1B, and TNFRSF1A; REG1A, COL6A3, and CST3; REG1A, COL6A3, and TNFRSF1A; REG1A, CST3, and TNFRSF1A; REG1B, COL6A3, and CST3; REG1B, COL6A3, and TNFRSF1A; REG1B, CST3, and TNFRSF1A; or COL6A3, CST3, and TNFRSF1A.
74. The method of any one of aspects 70 to 73, further comprising measuring one or more of COL28A1, UBE2G2, and REG1B.
75. A method comprising:
a) contacting a sample from a human subject with a set of capture reagents, wherein each capture reagent has affinity for a different protein of the set of proteins comprising three, four, five, six, seven, eight, nine or ten proteins selected from the group consisting of HAVCR1, FSTL3, RGMB, COL28A1, UBE2G2, REG1A, REG1B, COL6A3, CST3, and TNFRSF1A in a sample from a subject; and
b) measuring the level of each protein of the set of proteins with the set of capture reagents.
76. The method of aspect 75, wherein the set of capture reagents are selected from aptamers, antibodies and a combinations of aptamers and antibodies.
77. The method of aspect 75, wherein the sample is selected from blood, plasma, serum or urine.
78. The method of any one of aspects 75 to 77, wherein the method comprises measuring HAVCR1, FSTL3, and RGMB; HAVCR1, FSTL3, and COL28A1; HAVCR1, FSTL3, and UBE2G2; HAVCR1, FSTL3, and REG1A; HAVCR1, FSTL3, and REG1B; HAVCR1, FSTL3, and COL6A3; HAVCR1, FSTL3, and CST3; HAVCR1, FSTL3, and TNFRSF1; HAVCR1, RGMB, and COL28A1; HAVCR1, RGMB, and UBE2G2; HAVCR1, RGMB, and REG1A; HAVCR1, RGMB, and REG1B; HAVCR1, RGMB, and COL6A3; HAVCR1, RGMB, and CST3; HAVCR1, RGMB, and TNFRSF1A; HAVCR1, COL28A1, and UBE2G2; HAVCR1, COL28A1, and REG1A; HAVCR1, COL28A1, and REG1B; HAVCR1, COL28A1, and COL6A3; HAVCR1, COL28A1, and CST3; HAVCR1, COL28A1, and TNFRSF1A; HAVCR1, UBE2G2, and REG1A; HAVCR1, UBE2G2, and REG1B; HAVCR1, UBE2G2, and COL6A3; HAVCR1, UBE2G2, and CST3; HAVCR1, UBE2G2, and TNFRSF1A; HAVCR1, REG1A, and REG1B; HAVCR1, REG1A, and COL6A3; HAVCR1, REG1A, and CST3; HAVCR1, REG1A, and TNFRSF1A; HAVCR1, REG1B, and COL6A3; HAVCR1, REG1B, and CST3; HAVCR1, REG1B, and TNFRSF1A; HAVCR1, COL6A3, and CST3; HAVCR1, COL6A3, and TNFRSF1A; HAVCR1, CST3, and TNFRSF1A; FSTL3, RGMB, and COL28A1; FSTL3, RGMB, and UBE2G2; FSTL3, RGMB, and REG1A; FSTL3, RGMB, and REG1B; FSTL3, RGMB, and COL6A3; FSTL3, RGMB, and CST3; FSTL3, RGMB, and TNFRSF1A; FSTL3, COL28A1, and UBE2G2; FSTL3, COL28A1, and REG1A; FSTL3, COL28A1, and REG1B; FSTL3, COL28A1, and COL6A3; FSTL3, COL28A1, and CST3; FSTL3, COL28A1, and TNFRSF1A; FSTL3, UBE2G2, and REG1A; FSTL3, UBE2G2, and REG1B; FSTL3, UBE2G2, and COL6A3; FSTL3, UBE2G2, and CST3; FSTL3, UBE2G2, and TNFRSF1A; FSTL3, REG1A, and REG1B; FSTL3, REG1A, and COL6A3; FSTL3, REG1A, and CST3; FSTL3, REG1A, and TNFRSF1A; FSTL3, REG1B, and COL6A3; FSTL3, REG1B, and CST3; FSTL3, REG1B, and TNFRSF1A; FSTL3, COL6A3, and CST3; FSTL3, COL6A3, and TNFRSF1A; FSTL3, CST3, and TNFRSF1A; RGMB, COL28A1, and UBE2G2; RGMB, COL28A1, and REG1A; RGMB, COL28A1, and REG1B; RGMB, COL28A1, and COL6A3; RGMB, COL28A1, and CST3; RGMB, COL28A1, and TNFRSF1A; RGMB, UBE2G2, and REG1A; RGMB, UBE2G2, and REG1B; RGMB, UBE2G2, and COL6A3; RGMB, UBE2G2, and CST3; RGMB, UBE2G2, and TNFRSF1A; RGMB, REG1A, and REG1B; RGMB, REG1A, and COL6A3; RGMB, REG1A, and CST3; RGMB, REG1A, and TNFRSF1A; RGMB, REG1B, and COL6A3; RGMB, REG1B, and CST3; RGMB, REG1B, and TNFRSF1A; RGMB, COL6A3, and CST3; RGMB, COL6A3, and TNFRSF1A; RGMB, CST3, and TNFRSF1A; COL28A1, UBE2G2, and REG1A; COL28A1, UBE2G2, and REG1B; COL28A1, UBE2G2, and COL6A3; COL28A1, UBE2G2, and CST3; COL28A1, UBE2G2, and TNFRSF1A; COL28A1, REG1A, and REG1B; COL28A1, REG1A, and COL6A3; COL28A1, REG1A, and CST3; COL28A1, REG1A, and TNFRSF1A; COL28A1, REG1B, and COL6A3; COL28A1, REG1B, and CST3; COL28A1, REG1B, and TNFRSF1A; COL28A1, COL6A3, and CST3; COL28A1, COL6A3, and TNFRSF1A; COL28A1, CST3, and TNFRSF1A; UBE2G2, REG1A, and REG1B; UBE2G2, REG1A, and COL6A3; UBE2G2, REG1A, and CST3; UBE2G2, REG1A, and TNFRSF1A; UBE2G2, REG1B, and COL6A3; UBE2G2, REG1B, and CST3; UBE2G2, REG1B, and TNFRSF1A; UBE2G2, COL6A3, and CST3; UBE2G2, COL6A3, and TNFRSF1A; UBE2G2, CST3, and TNFRSF1A; REG1A, REG1B, and COL6A3; REG1A, REG1B, and CST3; REG1A, REG1B, and TNFRSF1A; REG1A, COL6A3, and CST3; REG1A, COL6A3, and TNFRSF1A; REG1A, CST3, and TNFRSF1A; REG1B, COL6A3, and CST3; REG1B, COL6A3, and TNFRSF1A; REG1B, CST3, and TNFRSF1A; or COL6A3, CST3, and TNFRSF1A.
79. The method of any one of aspects 75 to 78, further comprising measuring one or more of COL28A1, UBE2G2, and REG1B.
80. A method comprising:
a) measuring the level of HAVCR1 protein, and the level of at least one, two, three, four, five, six, seven, eight, or nine proteins selected from the group consisting of FSTL3, RGMB, COL28A1, UBE2G2, REG1A, REG1B, COL6A3, CST3, and TNFRSF1A in a sample from a human subject; and
b) identifying the human subject as being at relative risk for developing progressive chronic renal insufficiency within a 4 year period based on the level of HAVCR1 and the level of the at least one, two, three, four, five, six, seven, eight, or nine proteins.
81. A method comprising:
a) measuring the level of FSTL3 protein, and the level of at least one, two, three, four, five, six, seven, eight, or nine proteins selected from the group consisting of HAVCR1, RGMB, COL28A1, UBE2G2, REG1A, REG1B, COL6A3, CST3, and TNFRSF1A in a sample from a human subject; and
b) identifying the human subject as being at relative risk for developing progressive chronic renal insufficiency within a 4 year period based on the level of FSTL3 and the level of the at least one, two, three, four, five, six, seven, eight, or nine proteins.
82. A method comprising:
a) measuring the level of RGMB protein, and the level of at least one, two, three, four, five, six, seven, eight, or nine proteins selected from the group consisting of HAVCR1, FSTL3, COL28A1, UBE2G2, REG1A, REG1B, COL6A3, CST3, and TNFRSF1A in a sample from a human subject; and
b) identifying the human subject as being at relative risk for developing progressive chronic renal insufficiency within a 4 year period based on the level of RGMB and the level of the at least one, two, three, four, five, six, seven, eight, or nine proteins.
83. A method comprising:
a) measuring the level of REG1A protein, and the level of at least one, two, three, four, five, six, seven, eight, or nine proteins selected from the group consisting of HAVCR1, FSTL3, RGMB, COL28A1, UBE2G2, REG1B, COL6A3, CST3, and TNFRSF1A in a sample from a human subject; and
b) identifying the human subject as being at relative risk for developing progressive chronic renal insufficiency within a 4 year period based on the level of REG1A and the level of the at least one, two, three, four, five, six, seven, eight, or nine proteins.
84. A method comprising:
a) measuring the level of COL6A3 protein, and the level of at least one, two, three, four, five, six, seven, eight, or nine proteins selected from the group consisting of HAVCR1, FSTL3, RGMB, COL28A1, UBE2G2, REG1A, REG1B, CST3, and TNFRSF1A in a sample from a human subject; and
b) identifying the human subject as being at relative risk for developing progressive chronic renal insufficiency within a 4 year period based on the level of COL6A3 and the level of the at least one, two, three, four, five, six, seven, eight, or nine proteins.
85. A method comprising:
a) measuring the level of CST3 protein, and the level of at least one, two, three, four, five, six, seven, eight, or nine proteins selected from the group consisting of HAVCR1, FSTL3, RGMB, COL28A1, UBE2G2, REG1A, REG1B, COL6A3, and TNFRSF1A in a sample from a human subject; and
b) identifying the human subject as being at relative risk for developing progressive chronic renal insufficiency within a 4 year period based on the level of CST3 and the level of the at least one, two, three, four, five, six, seven, eight, or nine proteins.
86. A method comprising:
a) measuring the level of TNFRSF1A protein, and the level of at least one, two, three, four, five, six, seven, eight, or nine proteins selected from the group consisting of HAVCR1, FSTL3, RGMB, COL28A1, UBE2G2, REG1A, REG1B, COL6A3, and CST3 in a sample from a human subject; and
b) identifying the human subject as being at relative risk for developing progressive chronic renal insufficiency within a 4 year period based on the level of TNFRSF1A and the level of the at least one, two, three, four, five, six, seven, eight, or nine proteins.
87. The method of any one of aspects 80 to 86, wherein the measuring is performed using mass spectrometry, an aptamer based assay and/or an antibody based assay.
88. The method of any one of aspects 80 to 87, wherein the sample is selected from blood, plasma, serum or urine.
89. A method comprising:
a) contacting a sample from a human subject with a set of capture reagents, wherein each capture reagent has affinity for a different protein of the set of proteins comprising HAVCR1 protein, and at least one, two, three, four, five, six, seven, eight, or nine proteins selected from the group consisting of FSTL3, RGMB, COL28A1, UBE2G2, REG1A, REG1B, COL6A3, CST3, and TNFRSF1A; and
b) measuring the level of each protein of the set of proteins with the set of capture reagents.
90. A method comprising:
a) contacting a sample from a human subject with a set of capture reagents, wherein each capture reagent has affinity for a different protein of the set of proteins comprising FSTL3 protein, and at least one, two, three, four, five, six, seven, eight, or nine proteins selected from the group consisting of HAVCR1, RGMB, COL28A1, UBE2G2, REG1A, REG1B, COL6A3, CST3, and TNFRSF1A; and
b) measuring the level of each protein of the set of proteins with the set of capture reagents.
91. A method comprising:
a) contacting a sample from a human subject with a set of capture reagents, wherein each capture reagent has affinity for a different protein of the set of proteins comprising RGMB protein, and at least one, two, three, four, five, six, seven, eight, or nine proteins selected from the group consisting of HAVCR1, FSTL3, COL28A1, UBE2G2, REG1A, REG1B, COL6A3, CST3, and TNFRSF1A; and
b) measuring the level of each protein of the set of proteins with the set of capture reagents.
92. A method comprising:
a) contacting a sample from a human subject with a set of capture reagents, wherein each capture reagent has affinity for a different protein of the set of proteins comprising REG1A protein, and at least one, two, three, four, five, six, seven, eight, or nine proteins selected from the group consisting of HAVCR1, FSTL3, RGMB, COL28A1, UBE2G2, REG1B, COL6A3, CST3, and TNFRSF1A; and
b) measuring the level of each protein of the set of proteins with the set of capture reagents.
93. A method comprising:
a) contacting a sample from a human subject with a set of capture reagents, wherein each capture reagent has affinity for a different protein of the set of proteins comprising COL6A3 protein, and at least one, two, three, four, five, six, seven, eight, or nine proteins selected from the group consisting of HAVCR1, FSTL3, RGMB, COL28A1, UBE2G2, REG1A, REG1B, CST3, and TNFRSF1A; and
b) measuring the level of each protein of the set of proteins with the set of capture reagents.
94. A method comprising:
a) contacting a sample from a human subject with a set of capture reagents, wherein each capture reagent has affinity for a different protein of the set of proteins comprising CST3 protein, and at least one, two, three, four, five, six, seven, eight, or nine proteins selected from the group consisting of HAVCR1, FSTL3, RGMB, COL28A1, UBE2G2, REG1A, REG1B, COL6A3, and TNFRSF1A; and
b) measuring the level of each protein of the set of proteins with the set of capture reagents.
95. A method comprising:
a) contacting a sample from a human subject with a set of capture reagents, wherein each capture reagent has affinity for a different protein of the set of proteins comprising TNFRSF1A protein, and at least one, two, three, four, five, six, seven, eight, or nine proteins selected from the group consisting of HAVCR1, FSTL3, RGMB, COL28A1, UBE2G2, REG1A, REG1B, COL6A3, and CST3; and
b) measuring the level of each protein of the set of proteins with the set of capture reagents
96. The method of any one of aspects 89 to 95, wherein the set of capture reagents is selected from aptamers, antibodies and a combinations of aptamers and antibodies.
97. The method of any one of aspects 89 to 96, wherein the sample is selected from blood, plasma, serum or urine.
98. The method of any one of aspects 16 to 27, 32 to 41, 53 to 60, 75 to 79, and 89 to 97, further comprising identifying the human subject as being at relative risk for developing progressive chronic renal insufficiency within a 4 year period based on the level of each protein measured.
99. The method of any one of aspects 1 to 15, 28 to 31, 42 to 52, 61 to 74, 80 to 88, and 98, wherein the relative risk for developing progressive chronic renal insufficiency within a 4 year period is based on input of the level of each protein measured in a statistical model.
100. The method of aspect 99, wherein the model is a linear regression model.
101. The method of aspect 99 or 100, wherein the model has an area under the curve (AUC) selected from 0.65, 0.7, 0.75, 0.77, or greater.
102. The method of any one of aspects 99 to 101, where the model provides a binary prediction and/or a relative risk prediction for developing progressive chronic renal insufficiency within a 4 year period.

103. The method of any one of aspects 99 to 102, wherein the model is based on the level of each of the proteins selected from HAVCR1, FSTL3, RGMB, COL28A1, UBE2G2, REG1A, REG1B, COL6A3, CST3, and TNFRSF1A.
104. The method of aspect 103, wherein the model provides a binary prediction with a probability cut point X, wherein X<0.3, 0.31, 0.32, 0.32, 0.33, 0.34, 0.35, or 0.3533 predicts no risk for developing progressive chronic renal insufficiency within a 4 year period and X≥0.3, 0.31, 0.32, 0.33, 0.34, 0.35, or 0.3533 predicts risk for developing progressive chronic renal insufficiency within a 4 year period.
105. The method of aspect 104, wherein the risk of developing progressive chronic renal insufficiency indicates a risk of an event selected from a 50% decline in estimated glomerular filtration rate (eGFR); a diagnosis that kidney dialysis is needed; a development of eGFR<15 ml/min/1.73 m2; a development of end stage renal disease (ESRD); and a diagnosis that a kidney transplantation is needed.
106. The method of aspect 103, wherein the model provides the relative risk prediction for developing progressive chronic renal insufficiency within a 4 year period based on the level of each of the proteins selected from HAVCR1, FSTL3, RGMB, COL28A1, UBE2G2, REG1A, REG1B, COL6A3, CST3, and TNFRSF1A.
107. The method of aspect 98, wherein the level of each protein measured is determined from a relative florescence unit (RFU) or a protein concentration.
108. The method of aspect 103, wherein the model provides for a relative risk for developing progressive chronic renal insufficiency within a 4 year period.
109. The method of aspect 103, wherein the relative risk is selective from mild and severe.
110. The method of aspect 103, wherein the relative risk is a probability calculation.
111. The method of aspect 103, wherein the relative risk is a value range used to predict the development of progressive chronic renal insufficiency within a 4 year period.

### BRIEF DESCRIPTION OF THE DRAWINGS

FIG. 1 shows prognosis for developing end stage renal disease among CKD patients based on clinical parameters. G1-G5 corresponds to CKD stages 1-5.

FIG. 2 shows 95% CI of observed event rate by relative risk quintile in training data.

FIG. 3 shows a box plot of the predicted relative risk sorted by true PCRI class. The risk bin boundaries are shown as dashed lines.

FIG. 4 illustrates an exemplary computer system for use with various computer-implemented methods described herein.

FIG. 5 is a flowchart for a method of indicating evaluating risk of renal insufficiency in accordance with one embodiment.

### DETAILED DESCRIPTION

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

Unless defined otherwise, technical and scientific terms used herein have the same meaning as commonly understood by one of ordinary skill in the art to which this invention belongs. Although any methods, devices, and materials similar or equivalent to those described herein can be used in the practice or testing of the invention, the preferred methods, devices and materials are now described.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

As used in this application, including the appended claims, the singular forms “a,” “an,” and “the” include plural references, unless the content clearly dictates otherwise, and are used interchangeably with “at least one” and “one or more.” Thus, reference to “a SOMAmer” includes mixtures of SOMAmers, reference to “a probe” includes mixtures of probes, and the like.

As used herein, the term “about” represents an insignificant modification or variation of the numerical value such that the basic function of the item to which the numerical value relates is unchanged.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

The present application includes biomarkers, methods, devices, reagents, systems, and kits for the prediction of risk of renal insufficiency within a defined period of time, such as 4 years.

“Progressive Chronic Renal Insufficiency” or “PCRI” or “renal insufficiency” means a composite endpoint which is treated as a classification endpoint (yes/no within a given time frame) as defined by the development of at least one of the following within the time frame from test results:
a 50% decline in estimated glomerular filtration rate (eGFR),
a diagnosis that kidney dialysis is needed,
development of eGFR<15 ml/min/1.73 m²,
development of end stage renal disease (ESRD), or
a diagnosis that a kidney transplantation is needed.

“End Stage Renal Disease” or “ESRD” means that at least one of the following conditions are met: glomerular filtration rate is less than 15 ml/min/1.73 m2, chronic renal dialysis is needed, or kidney transplantation is needed.

“Relative risk” means the risk for developing PCRI in a given time frame as compared to the average risk in a reference population. The range for relative risk is 0.01-3.24. In one aspect, relative risk can be calculated

$$R R = \frac{p *}{q}$$

wherein p* is the probability that an individual develops PCRI within 4 years and q is the probability for the baseline individual in a training cohort.

“Biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), dried blood spots (e.g., obtained from infants), sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, bronchial brushing, synovial fluid, joint aspirate, organ secretions, cells, a cellular extract, and cerebrospinal fluid. This also includes experimentally separated fractions of all of the preceding. For example, a blood sample can be fractionated into serum, plasma or into fractions containing particular types of blood cells, such as red blood cells or white blood cells (leukocytes). If desired, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

Further, it should be realized that a biological sample can be derived by taking biological samples from a number of individuals and pooling them or pooling an aliquot of each individual's biological sample. The pooled sample can be treated as a sample from a single individual and if an increased or decreased risk of a renal insufficiency is established in the pooled sample, then each individual biological sample can be re-tested to determine which individual/s have an increased or decreased risk of a renal insufficiency.

As mentioned above, the biological sample can be urine. Urine samples provide certain advantages over blood or serum samples. Collecting blood or plasma samples through venipuncture is more complex than is desirable, can deliver variable volumes, can be worrisome for the patient, and involves some (small) risk of infection. Also, phlebotomy requires skilled personnel. The simplicity of collecting urine samples can lead to more widespread application of the subject methods.

For purposes of this specification, the phrase “data attributed to a biological sample from an individual” is intended to mean that the data in some form derived from, or were generated using, the biological sample of the individual. The data may have been reformatted, revised, or mathematically altered to some degree after having been generated, such as by conversion from units in one measurement system to units in another measurement system; but, the data are understood to have been derived from, or were generated using, the biological sample.

“Target”, “target molecule”, and “analyte” are used interchangeably herein to refer to any molecule of interest that may be present in a biological sample. A “molecule of interest” includes any minor variation of a particular molecule, such as, in the case of a protein, for example, minor variations in amino acid sequence, disulfide bond formation, glycosylation, lipidation, acetylation, phosphorylation, or any other manipulation or modification, such as conjugation with a labeling component, which does not substantially alter the identity of the molecule. A “target molecule”, “target”, or “analyte” is a set of copies of one type or species of molecule or multi-molecular structure. “Target molecules”, “targets”, and “analytes” refer to more than one such set of molecules. Exemplary target molecules include proteins, polypeptides, nucleic acids, carbohydrates, lipids, polysaccharides, glycoproteins, hormones, receptors, antigens, antibodies, affybodies, antibody mimics, viruses, pathogens, toxic substances, substrates, metabolites, transition state analogs, cofactors, inhibitors, drugs, dyes, nutrients, growth factors, cells, tissues, and any fragment or portion of any of the foregoing.

As used herein, “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can be single chains or associated chains. Also included within the definition are preproteins and intact mature proteins; peptides or polypeptides derived from a mature protein; fragments of a protein; splice variants; recombinant forms of a protein; protein variants with amino acid modifications, deletions, or substitutions; digests; and post-translational modifications, such as glycosylation, acetylation, phosphorylation, and the like.

As used herein, “marker” and “biomarker” and “feature” are used interchangeably to refer to a target molecule that indicates or is a sign of a normal or abnormal process in an individual or of a disease or other condition in an individual. More specifically, a “marker” or “biomarker” or “feature” is an anatomic, physiologic, biochemical, or molecular parameter associated with the presence of a specific physiological state or process, whether normal or abnormal, and, if abnormal, whether chronic or acute. Biomarkers are detectable and measurable by a variety of methods including laboratory assays and medical imaging. When a biomarker is a protein, it is also possible to use the expression of the corresponding gene as a surrogate measure of the amount or presence or absence of the corresponding protein biomarker in a biological sample or methylation state of the gene encoding the biomarker or proteins that control expression of the biomarker. In certain aspects, a feature is an analyte/SOMAmer reagent of other predictors in a statistical model.

As used herein, “biomarker value”, “value”, “biomarker level”, “feature level” and “level” are used interchangeably to refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The exact nature of the “value” or “level” depends on the specific design and components of the particular analytical method employed to detect the biomarker.

When a biomarker indicates or is a sign of an abnormal process or a disease or other condition in an individual, that biomarker is generally described as being either over-expressed or under-expressed as compared to an expression level or value of the biomarker that indicates or is a sign of a normal process or an absence of a disease or other condition in an individual. “Up-regulation”, “up-regulated”, “over-expression”, “over-expressed”, and any variations thereof are used interchangeably to refer to a value or level of a biomarker in a biological sample that is greater than a value or level (or range of values or levels) of the biomarker that is typically detected in similar biological samples from healthy or normal individuals. The terms may also refer to a value or level of a biomarker in a biological sample that is greater than a value or level (or range of values or levels) of the biomarker that may be detected at a different stage of a particular disease.

“Down-regulation”, “down-regulated”, “under-expression”, “under-expressed”, and any variations thereof are used interchangeably to refer to a value or level of a biomarker in a biological sample that is less than a value or level (or range of values or levels) of the biomarker that is typically detected in similar biological samples from healthy or normal individuals. The terms may also refer to a value or level of a biomarker in a biological sample that is less than a value or level (or range of values or levels) of the biomarker that may be detected at a different stage of a particular disease.

Further, a biomarker that is either over-expressed or under-expressed can also be referred to as being “differentially expressed” or as having a “differential level” or “differential value” as compared to a “normal” expression level or value of the biomarker that indicates or is a sign of a normal process or an absence of a disease or other condition in an individual. Thus, “differential expression” of a biomarker can also be referred to as a variation from a “normal” expression level of the biomarker.

The term “differential gene expression” and “differential expression” are used interchangeably to refer to a gene (or its corresponding protein expression product) whose expression is activated to a higher or lower level in a subject suffering from a specific disease or condition, relative to its expression in a normal or control subject. The terms also include genes (or the corresponding protein expression products) whose expression is activated to a higher or lower level at different stages of the same disease or condition. It is also understood that a differentially expressed gene may be either activated or inhibited at the nucleic acid level or protein level, or may be subject to alternative splicing to result in a different polypeptide product. Such differences may be evidenced by a variety of changes including mRNA levels, surface expression, secretion or other partitioning of a polypeptide. Differential gene expression may include a comparison of expression between two or more genes or their gene products; or a comparison of the ratios of the expression between two or more genes or their gene products; or even a comparison of two differently processed products of the same gene, which differ between normal subjects and subjects suffering from a disease; or between various stages of the same disease. Differential expression includes both quantitative, as well as qualitative, differences in the temporal or cellular expression pattern in a gene or its expression products among, for example, normal and diseased cells, or among cells which have undergone different disease events or disease stages.

As used herein, “individual” refers to a test subject or patient. The individual can be a mammal or a non-mammal. In various embodiments, the individual is a mammal. A mammalian individual can be a human or non-human. In various embodiments, the individual is a human. A healthy or normal individual is an individual in which the disease or condition of interest (including, for example, renal insufficiency) is not detectable by conventional diagnostic methods.

“Diagnose”, “diagnosing”, “diagnosis”, and variations thereof refer to the detection, determination, or recognition of a health status or condition of an individual on the basis of one or more signs, symptoms, data, or other information pertaining to that individual. The health status of an individual can be diagnosed as healthy/normal (i.e., a diagnosis of the absence of a disease or condition) or diagnosed as ill/abnormal (i.e., a diagnosis of the presence, or an assessment of the characteristics, of a disease or condition). The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the individual. The prediction of risk of a renal insufficiency includes distinguishing individuals who have an increased risk of renal insufficiency from individuals who do not.

“Prognose”, “prognosing”, “prognosis”, and variations thereof refer to the prediction of a future course of a disease or condition in an individual who has the disease or condition (e.g., predicting patient survival), and such terms encompass the evaluation of disease or condition response after the administration of a treatment or therapy to the individual.

“Evaluate”, “evaluating”, “evaluation”, and variations thereof encompass both “diagnose” and “prognose” and also encompass determinations or predictions about the future course of a disease or condition in an individual who does not have the disease as well as determinations or predictions regarding the risk that a disease or condition will recur in an individual who apparently has been cured of the disease or has had the condition resolved. The term “evaluate” also encompasses assessing an individual's response to a therapy, such as, for example, predicting whether an individual is likely to respond favorably to a therapeutic agent or is unlikely to respond to a therapeutic agent (or will experience toxic or other undesirable side effects, for example), selecting a therapeutic agent for administration to an individual, or monitoring or determining an individual's response to a therapy that has been administered to the individual. Thus, “evaluating” risk of renal insufficiency can include, for example, any of the following: predicting the future risk of renal insufficiency in an individual; predicting the risk of renal insufficiency in an individual who apparently has no renal insufficiency issues; or determining or predicting an individual's response to a renal insufficiency treatment or selecting a renal insufficiency treatment to administer to an individual based upon a determination of the biomarker values derived from the individual's biological sample. Evaluation of risk of renal insufficiency can include embodiments such as the assessment of risk of renal insufficiency on a continuous scale, or classification of risk of renal insufficiency in escalating classifications. Classification of risk includes, for example, classification into two or more classifications such as “No Elevated Risk of renal insufficiency” and “Elevated Risk of renal insufficiency.” The evaluation of risk of renal insufficiency is for a defined period; such period can be, for example, 4 years.

As used herein, “additional biomedical information” refers to one or more evaluations of an individual, other than using any of the biomarkers described herein, that are associated with renal insufficiency risk. “Additional biomedical information” includes any of the following: physical descriptors of an individual, including the height and/or weight of an individual; the age of an individual; the gender of an individual; change in weight; the ethnicity of an individual; occupational history; family history of renal insufficiency; the presence of a genetic marker(s) correlating with a higher risk of renal insufficiency in the individual; clinical symptoms such as abdominal pain, weight gain or loss gene expression values; physical descriptors of an individual, including physical descriptors observed by radiologic imaging; smoking status; alcohol use history; occupational history; dietary habits—salt, saturated fat and cholesterol intake; caffeine consumption; and imaging information. Testing of biomarker levels in combination with an evaluation of any additional biomedical information, including other laboratory tests, may, for example, improve sensitivity, specificity, and/or AUC for prediction of renal insufficiency as compared to biomarker testing alone or evaluating any particular item of additional biomedical information alone (e.g., carotid intima thickness imaging alone). Additional biomedical information can be obtained from an individual using routine techniques known in the art, such as from the individual themselves by use of a routine patient questionnaire or health history questionnaire, etc., or from a medical practitioner, etc. Testing of biomarker levels in combination with an evaluation of any additional biomedical information may, for example, improve sensitivity, specificity, and/or thresholds for prediction of renal insufficiency as compared to biomarker testing alone or evaluating any particular item of additional biomedical information alone (e.g., CT imaging alone).

As used herein, “detecting” or “determining” with respect to a biomarker value includes the use of both the instrument required to observe and record a signal corresponding to a biomarker value and the material/s required to generate that signal. In various embodiments, the biomarker value is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

“Solid support” refers herein to any substrate having a surface to which molecules may be attached, directly or indirectly, through either covalent or non-covalent bonds. A “solid support” can have a variety of physical formats, which can include, for example, a membrane; a chip (e.g., a protein chip); a slide (e.g., a glass slide or coverslip); a column; a hollow, solid, semi-solid, pore- or cavity-containing particle, such as, for example, a bead; a gel; a fiber, including a fiber optic material; a matrix; and a sample receptacle. Exemplary sample receptacles include sample wells, tubes, capillaries, vials, and any other vessel, groove or indentation capable of holding a sample. A sample receptacle can be contained on a multi-sample platform, such as a microtiter plate, slide, microfluidics device, and the like. A support can be composed of a natural or synthetic material, an organic or inorganic material. The composition of the solid support on which capture reagents are attached generally depends on the method of attachment (e.g., covalent attachment). Other exemplary receptacles include microdroplets and microfluidic controlled or bulk oil/aqueous emulsions within which assays and related manipulations can occur. Suitable solid supports include, for example, plastics, resins, polysaccharides, silica or silica-based materials, functionalized glass, modified silicon, carbon, metals, inorganic glasses, membranes, nylon, natural fibers (such as, for example, silk, wool and cotton), polymers, and the like. The material composing the solid support can include reactive groups such as, for example, carboxy, amino, or hydroxyl groups, which are used for attachment of the capture reagents. Polymeric solid supports can include, e.g., polystyrene, polyethylene glycol tetraphthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly)tetrafluoroethylene, (poly)vinylidenefluoride, polycarbonate, and polymethylpentene. Suitable solid support particles that can be used include, e.g., encoded particles, such as Luminex®-type encoded particles, magnetic particles, and glass particles.

As used herein, “adaptive normalization by maximum likelihood” means a process for normalizing the analytes to mitigate site bias.

As used herein, “analyte” is the protein target of a capture reagent. In certain aspects, the capture reagent is an aptamer. In certain further aspects, the capture reagent is a SOMAmer.

As used herein, “Lin's CCC” means concordance correlation coefficient which measures the concordance between a new test and an existing test that is considered the gold standard.

As used herein, “study”, means a set of samples and clinical data that are analyzed to derive the test.

As used herein, “training dataset”, means a subset of data from a study used to fit a model.

As used herein, “validation dataset”, means a final subset of data used to assess the performance of a final model developed on a verification dataset.

As used herein, “verification dataset”, means a separate subset of data used to provide an unbiased evaluation of a model fit on the training dataset while tuning model parameters.

As used herein, the term “need” or “needed” refers to a judgement made by a health care provider regarding treatment of a patient which is considered by the health care provider to be beneficial to the health status of the patient.

In one aspect, an objective kidney disease progression test is disclosed herein providing a model that predicts the development of at least one of the following within 4 years:
(1) a 50% decline in estimated glomerular filtration rate (eGFR),
(2) a diagnosis that kidney dialysis is needed,
(3) development of eGFR<15 ml/min/1.73 m2,
(4) development of end stage renal disease (ESRD), or
(5) a diagnosis that a kidney transplantation is needed.

In certain aspects, the composite endpoint provided by conditions (1)-(5) is treated as a classification endpoint (yes/no within a specified time frame) and is referred to herein as Progressive Chronic Renal Insufficiency (PCRI). In certain aspects, the specified time fame is 4 years.

In certain aspects, a test was developed using the Chronic Renal Insufficiency Cohort (CRIC) which was split into training (70%), verification (15%), and validation (15%) datasets.

TABLE 3
AUC and 95% CI for training, verification, and validation data

| Dataset | n | KFRE AUC | Model AUC | AUC (95% CI) |
|---|---|---|---|---|
| Training | 2315 | 0.77 | 0.82 | (0.80, 0.84) |
| Verification | 496 | (95% CI: | 0.82 | (0.78, 0.86) |
| Validation | 494 | 0.75, 0.78) | 0.79 | (0.74, 0.82) |

Results from CRIC have resulted in more than 200 peer-reviewed publications and significant contributions to understanding CKD progression. (Hannan M, Ansari S, Meza N, et al. Risk Factors for CKD Progression: Overview of Findings from the CRIC Study. Clin J Am Soc Nephrol 2020; 11:07830520). The components of the PCRI composite endpoint include the most relevant clinical characteristics that describe progression of chronic kidney disease.

Kidney disease progression tests disclosed herein are effective for testing adults diagnosed with mild to severe chronic kidney disease as defined in Table 1.

In certain aspects, a logistic regression model is disclosed with 10 features and an optimal probability cut point of 0.3533 for PCRI (≥0.3533 is PCRI=yes, <0.3533 is PCRI=no). The model output may be reported as Relative Risk (RR) for developing PCRI in 4 years, as compared to the average risk in the reference population. The range of RR is 0.01-3.24.

In certain aspects, the minimum performance requirement for a kidney disease progression test is an area under the curve (AUC) at least equivalent to the Kidney Failure Risk Equation (KFRE) as applied to the CRIC data set (AUC=0.77, 95% CI: 0.75, 0.78). Validation exceeds the performance metric of an AUC≥0.77.

In certain aspects, the kidney disease progression test is intended for use based on medical necessity for an individual patient with diagnosed chronic kidney disease of any stage. In certain further aspects, results are reported as a relative risk in relation to a reference population of patients with chronic kidney disease that experience the composite endpoint at an average rate of 27% within 4 years. In certain aspects, the reference population age ranged from 23-75 years, CKD Stages I-V (80% Stage III-IV) and an eGFR range of 10.6-86.4 ml/min/1.73 m² and relative risks reported can range from 0.01 to 3.24.

In certain aspects, the kidney disease progression test is applied in a research context to predict the development of PCRI within four years. In certain further aspects, in the research context, benefits and risks pertain to decision making in research studies for participant monitoring, stratification, and enrichment.

In certain aspects, validation was performed using EDTA plasma, samples from individuals with diagnosed chronic kidney disease and an eGFR range of 10.6-86.4 ml/min/1.73 m², and samples from individuals from multiple races/ethnicities living in North America, aged 23-75 years. In certain aspects, the sample matrix is human EDTA plasma.

The risk analysis profile may be described as in Table 4.

TABLE 4
Risk analysis

| | | | | Need for |
|---|---|---|---|---|
| | | Likelihood | | Mitigating |
| Hazard | Hazardous Situation(s) | relative to SoC | Harm(s) | Measures |
| False Positive | HCP recommends | [ ] Lower | Common side effects from | [✓] Low |
| Result | unneeded medical | [✓] Equivalent | drugs including headache, | [ ] Med |
| (erroneously | management for kidney | [ ] Higher | nausea, etc. | [ ] High |
| predict PCRI) | disease | | | |
| | HCP refers patient for | | Unneeded health care | [✓] Low |
| | specialist consultation | | visit | [ ] Med |
| | | | | [ ] High |
| | HCP recommends further | | Unneeded diagnostic | [✓] Low |
| | work up to rule in/out | | tests | [ ] Med |
| | PCRI | | | [ ] High |
| False Negative | Delay in identification of | [ ] Lower | Delayed additional | [ ] Low |
| Result | PCRI risk until later in the | [✓] Equivalent | diagnostic tests | [✓] Med |
| (erroneously do | disease | [ ] Higher | | [ ] High |
| not predict | | | Delayed initiation of renal | [ ] Low |
| PCRI) | | | protective therapies, | [✓] Med |
| | | | including blood pressure | [ ] High |
| | | | management and SGLT2i | |
| HCP | Patient thinks risk for | [ ] Lower | Delay in pursuing | [ ] Low |
| misinterprets | PCRI is lower than their | [ ] Equivalent | necessary additional | [✓] Med |
| relative risk as | actual risk | [✓] Higher | diagnostic tests | [ ] High |
| lower than | | | Delay in initiation of | |
| actual risk | | | kidney-sparing | |
| | | | medications | |
| HCP | Patient thinks risk for | [ ] Lower | Unneeded additional | [ ] Low |
| misinterprets | PCRI is higher than their | [ ] Equivalent | diagnostic tests | [✓] Med |
| relative risk as | actual risk | [✓] Higher | Unneeded initiation of | [ ] High |
| higher than | | | kidney-sparing | |
| actual risk | | | medications | |
| Off label use | HCP uses test to | [ ] Lower | Unneeded treatment for | [✓] Low |
| | diagnose PCRI | [✓] Equivalent | ESRD | [ ] Med |
| | | [ ] Higher | | [ ] High |
| | HCP uses test in a | | Unneeded worry and | [✓] Low |
| | patient who is not part of | | diagnostics tests for a | [ ] Med |
| | the intended use | | patient who does not | [ ] High |
| | population | | have/is not at risk for | |
| | | | kidney disease | |

The testing methods disclosed herein provide convenience for health care providers in assessment and monitoring of the risk for PCRI and help to identify patients who are at risk for developing PCRI earlier in the process, when interventions may delay or prevent the progression to end stage renal disease (ESRD).

The Chronic Renal Insufficiency Cohort (CRIC) is enriched with later stages of kidney disease (85% stage 3a or later) compared to the distribution of kidney disease stages in the US population of CKD patients (˜50% stage 3a or later). For this reason, the incidence of ESRD is higher in CRIC when compared to the US population of CKD patients. The tests disclosed herein can be supplemented for improved accuracy by, additional assessments include but are not limited to health status, including comorbid conditions such as diabetes, clinical pathology, clinical laboratory tests (e.g., eGFR and albuminuria) renal imaging, and histology to assess risk of ESRD and to mitigate the risk of a false test result.

TABLE 5
Model Performance Requirement

| Validation Metric(s) | Performance Requirement |
|---|---|
| Area Under the Curve (AUC) | Model AUC ≥ 0.77 |

The performance threshold was established based on the performance of the KFRE. Accuracy, sensitivity and specificity are calculated but are not part of the performance requirement threshold. Sensitivity and specificity depend on where the cut point is placed on the receiver operating curve.

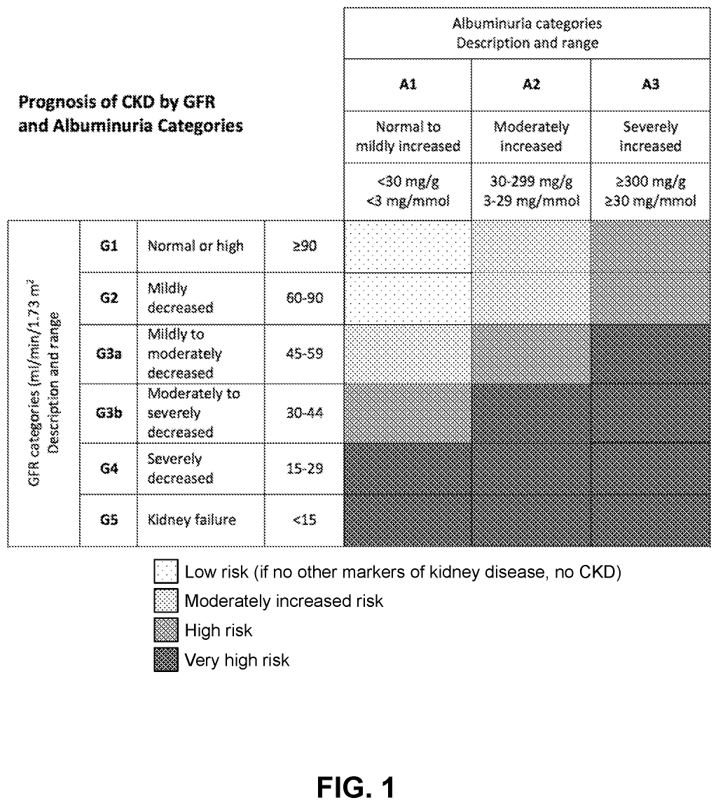

The KFRE is an equation commonly used in clinical practice to predict the risk of CKD progression to ESRD. This equation has been validated in a cohort of patients with all stages of CKD (Stages 1-5) (Major R W, Shepherd D, Medcalf J F, et al. The Kidney Failure Risk Equation for prediction of end stage renal disease in UK primary care: An external validation and clinical impact projection cohort study. PLoS Med 2019; 16:e1002955. doi: 1002910.1001371/journal.pmed.1002955. eCollection 1002019 November) so it was applied to an entire cohort to ensure a valid comparison. In addition, the measured components of the KFRE (eGFR and proteinuria) were used to evaluate kidney function at all stages of CKD (FIG. 1).

When the KFRE was applied to the subset of the CRIC population that is comparable to the one used for development of the KFRE (i.e., stage 3a or worse and ESRD as the outcome) an AUC of 0.83 was obtained, which agrees with the published validation AUC from the initial development of the KFRE, which was 0.83. This result suggests that the performance of this equation in the tested cohort is comparable to its published performance (in other cohorts).

The model performance requirement (AUC≥0.77) is based on the performance of the KFRE (Eq 1) in the full CRIC dataset, which includes individuals earlier in the disease process and an expanded endpoint definition in order to identify individuals at risk for progression at a point when medical intervention may slow the loss of kidney function.

To demonstrate test comparability to the KFRE equation as developed, the AUC of the proteomic model was analyzed and KFRE in the subset of the population and outcome that is comparable to the population initially used to develop the KFRE (Table 6). While this analysis is not linked to a performance requirement, it is presented here to demonstrate that the Kidney Failure Prognosis Test is comparable to the KFRE in the population used to derive the KFRE equation as well as the expanded intended use population for this test.

TABLE 6
Comparison of AUC's using different cohort subsets and endpoints

| | Portion of cohort | Endpoint | KFRE | Proteomic |
|---|---|---|---|---|
| Dataset | analyzed | analyzed | AUC | model AUC |
| Training | Entire cohort | PCRI | 0.77 | 0.82 |
| | CKD Stages 3-5 | ESRD only | 0.83 | 0.84 |
| Verification | Entire cohort | PCRI | 0.77 | 0.82 |
| | CKD Stages 3-5 | ESRD only | 0.84 | 0.81 |

Eq 1: The kidney failure risk equation

$$Pr ( E S R D ) = 100 * ( 1 - {0.924}^{exp [ ( 0.2694 ( male ) - 0.2167 ( age / 10 ) - 0.55418 ( eGFR / 5 ) + 0.45608 ( ln ) 24 hour proeinuria in mg / g ) ) ) + 2.96774 ]} )$$

$$Where :$$

$$Pr ( E S R D ) = probability of end renal disease$$

$$e G F R = estimated glomerular filtration rate ( in ml / min / 1.73 {m}^{2} )$$

In one aspect, one or more biomarkers are provided for use either alone or in various combinations to evaluate the risk of a renal insufficiency within a 4 year time period. As described in detail below, exemplary embodiments include the biomarkers provided in Table 8, which were identified using a multiplex SOMAmer-based assay.

In a preferred embodiment, the model has 10 features (Table 8) and predicts PCRI in four years. The model output is a relative risk for PCRI compared to an average person with CKD. The range of RR's is 0.01-3.24. Validation exceeds the performance metric of an AUC>0.77.

In one embodiment, the number of biomarkers useful for a biomarker subset or panel is based on the sensitivity and specificity value for the particular combination of biomarker values. The terms “sensitivity” and “specificity” are used herein with respect to the ability to correctly classify an individual, based on one or more biomarker values detected in their biological sample, as having an increased risk of having a renal insufficiency within 4 years or not having increased relative risk of having renal insufficiency within the same time period. “Sensitivity” indicates the performance of the biomarker(s) with respect to correctly classifying individuals that have increased risk of renal insufficiency. “Specificity” indicates the performance of the biomarker(s) with respect to correctly classifying individuals who do not have increased relative risk of renal insufficiency.

In an alternate method, scores may be reported on a continuous range, with a threshold of high, intermediate or low risk of renal insufficiency, with thresholds determined based on clinical findings.

Another factor that can affect the number of biomarkers to be used in a subset or panel of biomarkers is the procedures used to obtain biological samples from individuals who are being assessed for risk of renal insufficiency. In a carefully controlled sample procurement environment, the number of biomarkers necessary to meet desired sensitivity and specificity and/or threshold values will be lower than in a situation where there can be more variation in sample collection, handling and storage.

### Exemplary Uses of Biomarkers

In various exemplary embodiments, methods are provided for evaluating risk of renal insufficiency in an individual by detecting one or more biomarker values corresponding to one or more biomarkers that are present in the circulation of an individual, such as in serum or plasma, by any number of analytical methods, including any of the analytical methods described herein. These biomarkers are, for example, differentially expressed in individuals with increased risk of renal insufficiency as compared to individuals without increased risk of renal insufficiency. Detection of the differential expression of a biomarker in an individual can be used, for example, to permit the prediction of risk of renal insufficiency within 4 year time frame.

In addition to testing biomarker levels as a stand-alone diagnostic test, biomarker levels can also be done in conjunction with determination of SNPs or other genetic lesions or variability that are indicative of increased risk of susceptibility of disease or condition. (See, e.g., Amos et al., Nature Genetics 40, 616-622 (2009)).

In addition to testing biomarker levels as a stand-alone diagnostic test, biomarker levels can also be used in conjunction with radiologic screening. Biomarker levels can also be used in conjunction with relevant symptoms or genetic testing. Detection of any of the biomarkers described herein may be useful after the risk of renal insufficiency has been evaluated to guide appropriate clinical care of the individual, including increasing to more aggressive levels of care in high risk individuals after the renal insufficiency risk has been determined. In addition to testing biomarker levels in conjunction with relevant symptoms or risk factors, information regarding the biomarkers can also be evaluated in conjunction with other types of data, particularly data that indicates an individual's risk for renal insufficiency (e.g., patient clinical history, symptoms, family history, history of smoking or alcohol use, risk factors such as the presence of a genetic marker(s), and/or status of other biomarkers, etc.). These various data can be assessed by automated methods, such as a computer program/software, which can be embodied in a computer or other apparatus/device.

In addition to testing biomarker levels in conjunction with radiologic screening in high risk individuals (e.g., assessing biomarker levels in conjunction with blockage detected in a coronary angiogram), information regarding the biomarkers can also be evaluated in conjunction with other types of data, particularly data that indicates an individual's risk for developing renal insufficiency (e.g., patient clinical history, symptoms, family history of renal disease, risk factors such as whether or not the individual is a smoker, heavy alcohol user and/or status of other biomarkers, etc.). These various data can be assessed by automated methods, such as a computer program/software, which can be embodied in a computer or other apparatus/device.

Any of the described biomarkers may also be used in imaging tests. For example, an imaging agent can be coupled to any of the described biomarkers, which can be used to aid in prediction of risk of renal insufficiency, to monitor response to therapeutic interventions, to select for target populations in a clinical trial among other uses.

### Detection and Determination of Biomarkers and Biomarker Values

A biomarker value for the biomarkers described herein can be detected using any of a variety of known analytical methods. In one embodiment, a biomarker value is detected using a capture reagent. As used herein, a “capture agent” or “capture reagent” refers to a molecule that is capable of binding specifically to a biomarker. In various embodiments, the capture reagent can be exposed to the biomarker in solution or can be exposed to the biomarker while the capture reagent is immobilized on a solid support. In other embodiments, the capture reagent contains a feature that is reactive with a secondary feature on a solid support. In these embodiments, the capture reagent can be exposed to the biomarker in solution, and then the feature on the capture reagent can be used in conjunction with the secondary feature on the solid support to immobilize the biomarker on the solid support. The capture reagent is selected based on the type of analysis to be conducted. Capture reagents include but are not limited to SOMAmers, antibodies, adnectins, ankyrins, other antibody mimetics and other protein scaffolds, autoantibodies, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, imprinted polymers, avimers, peptidomimetics, a hormone receptor, a cytokine receptor, and synthetic receptors, and modifications and fragments of these.

In some embodiments, a biomarker value is detected using a biomarker/capture reagent complex.

In other embodiments, the biomarker value is derived from the biomarker/capture reagent complex and is detected indirectly, such as, for example, as a result of a reaction that is subsequent to the biomarker/capture reagent interaction, but is dependent on the formation of the biomarker/capture reagent complex.

In some embodiments, the biomarker value is detected directly from the biomarker in a biological sample.

In one embodiment, the biomarkers are detected using a multiplexed format that allows for the simultaneous detection of two or more biomarkers in a biological sample. In one embodiment of the multiplexed format, capture reagents are immobilized, directly or indirectly, covalently or non-covalently, in discrete locations on a solid support. In another embodiment, a multiplexed format uses discrete solid supports where each solid support has a unique capture reagent associated with that solid support, such as, for example quantum dots. In another embodiment, an individual device is used for the detection of each one of multiple biomarkers to be detected in a biological sample. Individual devices can be configured to permit each biomarker in the biological sample to be processed simultaneously. For example, a microtiter plate can be used such that each well in the plate is used to uniquely analyze one of multiple biomarkers to be detected in a biological sample.

In one or more of the foregoing embodiments, a fluorescent tag can be used to label a component of the biomarker/capture complex to enable the detection of the biomarker value. In various embodiments, the fluorescent label can be conjugated to a capture reagent specific to any of the biomarkers described herein using known techniques, and the fluorescent label can then be used to detect the corresponding biomarker value. Suitable fluorescent labels include rare earth chelates, fluorescein and its derivatives, rhodamine and its derivatives, dansyl, allophycocyanin, PBXL-3, Qdot 605, Lissamine, phycoerythrin, Texas Red, and other such compounds.

In one embodiment, the fluorescent label is a fluorescent dye molecule. In some embodiments, the fluorescent dye molecule includes at least one substituted indolium ring system in which the substituent on the 3-carbon of the indolium ring contains a chemically reactive group or a conjugated substance. In some embodiments, the dye molecule includes an AlexFluor molecule, such as, for example, AlexaFluor 488, AlexaFluor 532, AlexaFluor 647, AlexaFluor 680, or AlexaFluor 700. In other embodiments, the dye molecule includes a first type and a second type of dye molecule, such as, e.g., two different AlexaFluor molecules. In other embodiments, the dye molecule includes a first type and a second type of dye molecule, and the two dye molecules have different emission spectra.

Fluorescence can be measured with a variety of instrumentation compatible with a wide range of assay formats. For example, spectrofluorimeters have been designed to analyze microtiter plates, microscope slides, printed arrays, cuvettes, etc. See Principles of Fluorescence Spectroscopy, by J. R. Lakowicz, Springer Science+Business Media, Inc., 2004. See Bioluminescence & Chemiluminescence: Progress & Current Applications; Philip E. Stanley and Larry J. Kricka editors, World Scientific Publishing Company, January 2002.

In one or more of the foregoing embodiments, a chemiluminescence tag can optionally be used to label a component of the biomarker/capture complex to enable the detection of a biomarker value. Suitable chemiluminescent materials include any of oxalyl chloride, Rodamin 6G, Ru(bipy)32+, TMAE (tetrakis(dimethylamino)ethylene), Pyrogallol (1,2,3-trihydroxibenzene), Lucigenin, peroxyoxalates, Aryl oxalates, Acridinium esters, dioxetanes, and others.

In yet other embodiments, the detection method includes an enzyme/substrate combination that generates a detectable signal that corresponds to the biomarker value. Generally, the enzyme catalyzes a chemical alteration of the chromogenic substrate which can be measured using various techniques, including spectrophotometry, fluorescence, and chemiluminescence. Suitable enzymes include, for example, luciferases, luciferin, malate dehydrogenase, urease, horseradish peroxidase (HRPO), alkaline phosphatase, beta-galactosidase, glucoamylase, lysozyme, glucose oxidase, galactose oxidase, and glucose-6-phosphate dehydrogenase, uricase, xanthine oxidase, lactoperoxidase, microperoxidase, and the like.

In yet other embodiments, the detection method can be a combination of fluorescence, chemiluminescence, radionuclide or enzyme/substrate combinations that generate a measurable signal. Multimodal signaling could have unique and advantageous characteristics in biomarker assay formats.

More specifically, the biomarker values for the biomarkers described herein can be detected using known analytical methods including, singleplex SOMAmer assays, multiplexed SOMAmer assays, singleplex or multiplexed immunoassays, mRNA expression profiling, miRNA expression profiling, mass spectrometric analysis, histological/cytological methods, etc. as detailed below.

### Determination of Biomarker Values Using SOMAmer-Based Assays

Assays directed to the detection and quantification of physiologically significant molecules in biological samples and other samples are important tools in scientific research and in the health care field. One class of such assays involves the use of a microarray that includes one or more aptamers immobilized on a solid support. The aptamers are each capable of binding to a target molecule in a highly specific manner and with very high affinity. See, e.g., U.S. Pat. No. 5,475,096 entitled “Nucleic Acid Ligands”; see also, e.g., U.S. Pat. Nos. 6,242,246, 6,458,543, and 6,503,715, each of which is entitled “Nucleic Acid Ligand Diagnostic Biochip”. Once the microarray is contacted with a sample, the aptamers bind to their respective target molecules present in the sample and thereby enable a determination of a biomarker value corresponding to a biomarker.

As used herein, an “aptamer” refers to a nucleic acid that has a specific binding affinity for a target molecule. It is recognized that affinity interactions are a matter of degree; however, in this context, the “specific binding affinity” of an aptamer for its target means that the aptamer binds to its target generally with a much higher degree of affinity than it binds to other components in a test sample. An “aptamer” is a set of copies of one type or species of nucleic acid molecule that has a particular nucleotide sequence. An aptamer can include any suitable number of nucleotides, including any number of chemically modified nucleotides. “Aptamers” refers to more than one such set of molecules. Different aptamers can have either the same or different numbers of nucleotides. Aptamers can be DNA or RNA or chemically modified nucleic acids and can be single stranded, double stranded, or contain double stranded regions, and can include higher ordered structures. An aptamer can also be a photoaptamer, where a photoreactive or chemically reactive functional group is included in the aptamer to allow it to be covalently linked to its corresponding target. Any of the aptamer methods disclosed herein can include the use of two or more aptamers that specifically bind the same target molecule. As further described below, an aptamer may include a tag. If an aptamer includes a tag, all copies of the aptamer need not have the same tag. Moreover, if different aptamers each include a tag, these different aptamers can have either the same tag or a different tag.

An aptamer can be identified using any known method, including the SELEX process. Once identified, an aptamer can be prepared or synthesized in accordance with any known method, including chemical synthetic methods and enzymatic synthetic methods.

As used herein, a “SOMAmer” or Slow Off-Rate Modified Aptamer refers to an aptamer having improved off-rate characteristics. SOMAmers can be generated using the improved SELEX methods described in U.S. Publication No. 2009/0004667, entitled “Method for Generating Aptamers with Improved Off-Rates.”

The terms “SELEX” and “SELEX process” are used interchangeably herein to refer generally to a combination of (1) the selection of aptamers that interact with a target molecule in a desirable manner, for example binding with high affinity to a protein, with (2) the amplification of those selected nucleic acids. The SELEX process can be used to identify aptamers with high affinity to a specific target or biomarker.

SELEX generally includes preparing a candidate mixture of nucleic acids, binding of the candidate mixture to the desired target molecule to form an affinity complex, separating the affinity complexes from the unbound candidate nucleic acids, separating and isolating the nucleic acid from the affinity complex, purifying the nucleic acid, and identifying a specific aptamer sequence. The process may include multiple rounds to further refine the affinity of the selected aptamer. The process can include amplification steps at one or more points in the process. See, e.g., U.S. Pat. No. 5,475,096, entitled “Nucleic Acid Ligands”. The SELEX process can be used to generate an aptamer that covalently binds its target as well as an aptamer that non-covalently binds its target. See, e.g., U.S. Pat. No. 5,705,337 entitled “Systematic Evolution of Nucleic Acid Ligands by Exponential Enrichment: Chemi-SELEX.”

The SELEX process can be used to identify high-affinity aptamers containing modified nucleotides that confer improved characteristics on the aptamer, such as, for example, improved in vivo stability or improved delivery characteristics. Examples of such modifications include chemical substitutions at the ribose and/or phosphate and/or base positions. SELEX process-identified aptamers containing modified nucleotides are described in U.S. Pat. No. 5,660,985, entitled “High Affinity Nucleic Acid Ligands Containing Modified Nucleotides”, which describes oligonucleotides containing nucleotide derivatives chemically modified at the 5′- and 2′-positions of pyrimidines. U.S. Pat. No. 5,580,737, see supra, describes highly specific aptamers containing one or more nucleotides modified with 2′-amino (2′-NH2), 2′-fluoro (2′-F), and/or 2′-O-methyl (2′-OMe). See also, U.S. Patent Application Publication 20090098549, entitled “SELEX and PHOTOSELEX”, which describes nucleic acid libraries having expanded physical and chemical properties and their use in SELEX and photoSELEX.

SELEX can also be used to identify aptamers that have desirable off-rate characteristics. See U.S. Patent Application Publication 20090004667, entitled “Method for Generating Aptamers with Improved Off-Rates”, which describes improved SELEX methods for generating aptamers that can bind to target molecules. As mentioned above, these slow off-rate aptamers are known as “SOMAmers.” Methods for producing aptamers or SOMAmers and photoaptamers or SOMAmers having slower rates of dissociation from their respective target molecules are described. The methods involve contacting the candidate mixture with the target molecule, allowing the formation of nucleic acid-target complexes to occur, and performing a slow off-rate enrichment process wherein nucleic acid-target complexes with fast dissociation rates will dissociate and not reform, while complexes with slow dissociation rates will remain intact. Additionally, the methods include the use of modified nucleotides in the production of candidate nucleic acid mixtures to generate aptamers or SOMAmers with improved off-rate performance.

A variation of this assay employs aptamers that include photoreactive functional groups that enable the aptamers to covalently bind or “photocrosslink” their target molecules. See, e.g., U.S. Pat. No. 6,544,776 entitled “Nucleic Acid Ligand Diagnostic Biochip”. These photoreactive aptamers are also referred to as photoaptamers. See, e.g., U.S. Pat. Nos. 5,763,177, 6,001,577, and 6,291,184, each of which is entitled “Systematic Evolution of Nucleic Acid Ligands by Exponential Enrichment: Photoselection of Nucleic Acid Ligands and Solution SELEX”; see also, e.g., U.S. Pat. No. 6,458,539, entitled “Photoselection of Nucleic Acid Ligands”. After the microarray is contacted with the sample and the photoaptamers have had an opportunity to bind to their target molecules, the photoaptamers are photoactivated, and the solid support is washed to remove any non-specifically bound molecules. Harsh wash conditions may be used, since target molecules that are bound to the photoaptamers are generally not removed, due to the covalent bonds created by the photoactivated functional group(s) on the photoaptamers. In this manner, the assay enables the detection of a biomarker value corresponding to a biomarker in the test sample.

In both of these assay formats, the aptamers or SOMAmers are immobilized on the solid support prior to being contacted with the sample. Under certain circumstances, however, immobilization of the aptamers or SOMAmers prior to contact with the sample may not provide an optimal assay. For example, pre-immobilization of the aptamers or SOMAmers may result in inefficient mixing of the aptamers or SOMAmers with the target molecules on the surface of the solid support, perhaps leading to lengthy reaction times and, therefore, extended incubation periods to permit efficient binding of the aptamers or SOMAmers to their target molecules. Further, when photoaptamers or photoSOMAmers are employed in the assay and depending upon the material utilized as a solid support, the solid support may tend to scatter or absorb the light used to effect the formation of covalent bonds between the photoaptamers or photoSOMAmers and their target molecules. Moreover, depending upon the method employed, detection of target molecules bound to their aptamers or photoSOMAmers can be subject to imprecision, since the surface of the solid support may also be exposed to and affected by any labeling agents that are used. Finally, immobilization of the aptamers or SOMAmers on the solid support generally involves an aptamer or SOMAmer-preparation step (i.e., the immobilization) prior to exposure of the aptamers or SOMAmers to the sample, and this preparation step may affect the activity or functionality of the aptamers or SOMAmers.

SOMAmer assays that permit a SOMAmer to capture its target in solution and then employ separation steps that are designed to remove specific components of the SOMAmer-target mixture prior to detection have also been described (see U.S. Patent Application Publication 20090042206, entitled “Multiplexed Analyses of Test Samples”). The described SOMAmer assay methods enable the detection and quantification of a non-nucleic acid target (e.g., a protein target) in a test sample by detecting and quantifying a nucleic acid (i.e., a SOMAmer). The described methods create a nucleic acid surrogate (i.e, the SOMAmer) for detecting and quantifying a non-nucleic acid target, thus allowing the wide variety of nucleic acid technologies, including amplification, to be applied to a broader range of desired targets, including protein targets.

SOMAmers can be constructed to facilitate the separation of the assay components from a SOMAmer biomarker complex (or photoSOMAmer biomarker covalent complex) and permit isolation of the SOMAmer for detection and/or quantification. In one embodiment, these constructs can include a cleavable or releasable element within the SOMAmer sequence. In other embodiments, additional functionality can be introduced into the SOMAmer, for example, a labeled or detectable component, a spacer component, or a specific binding tag or immobilization element. For example, the SOMAmer can include a tag connected to the SOMAmer via a cleavable moiety, a label, a spacer component separating the label, and the cleavable moiety. In one embodiment, a cleavable element is a photocleavable linker. The photocleavable linker can be attached to a biotin moiety and a spacer section, can include an NHS group for derivatization of amines, and can be used to introduce a biotin group to a SOMAmer, thereby allowing for the release of the SOMAmer later in an assay method.

Homogenous assays, done with all assay components in solution, do not require separation of sample and reagents prior to the detection of signal. These methods are rapid and easy to use. These methods generate signal based on a molecular capture or binding reagent that reacts with its specific target. For prediction of renal insufficiency, the molecular capture reagents would be a SOMAmer or an antibody or the like and the specific target would be a renal insufficiency biomarker as in Table 8.

In one embodiment, a method for signal generation takes advantage of anisotropy signal change due to the interaction of a fluorophore-labeled capture reagent with its specific biomarker target. When the labeled capture reagent reacts with its target, the increased molecular weight causes the rotational motion of the fluorophore attached to the complex to become much slower changing the anisotropy value. By monitoring the anisotropy change, binding events may be used to quantitatively measure the biomarkers in solutions. Other methods include fluorescence polarization assays, molecular beacon methods, time resolved fluorescence quenching, chemiluminescence, fluorescence resonance energy transfer, and the like.

An exemplary solution-based SOMAmer assay that can be used to detect a biomarker value corresponding to a biomarker in a biological sample includes the following: (a) preparing a mixture by contacting the biological sample with a SOMAmer that includes a first tag and has a specific affinity for the biomarker, wherein a SOMAmer affinity complex is formed when the biomarker is present in the sample; (b) exposing the mixture to a first solid support including a first capture element, and allowing the first tag to associate with the first capture element; (c) removing any components of the mixture not associated with the first solid support; (d) attaching a second tag to the biomarker component of the SOMAmer affinity complex; (e) releasing the SOMAmer affinity complex from the first solid support; (f) exposing the released SOMAmer affinity complex to a second solid support that includes a second capture element and allowing the second tag to associate with the second capture element; (g) removing any non-complexed SOMAmer from the mixture by partitioning the non-complexed SOMAmer from the SOMAmer affinity complex; (h) eluting the SOMAmer from the solid support; and (i) detecting the biomarker by detecting the SOMAmer component of the SOMAmer affinity complex.

Any means known in the art can be used to detect a biomarker value by detecting the SOMAmer component of a SOMAmer affinity complex. A number of different detection methods can be used to detect the SOMAmer component of an affinity complex, such as, for example, hybridization assays, mass spectroscopy, or QPCR. In some embodiments, nucleic acid sequencing methods can be used to detect the SOMAmer component of a SOMAmer affinity complex and thereby detect a biomarker value. Briefly, a test sample can be subjected to any kind of nucleic acid sequencing method to identify and quantify the sequence or sequences of one or more SOMAmers present in the test sample. In some embodiments, the sequence includes the entire SOMAmer molecule or any portion of the molecule that may be used to uniquely identify the molecule. In other embodiments, the identifying sequencing is a specific sequence added to the SOMAmer; such sequences are often referred to as “tags,” “barcodes,” or “zipcodes.” In some embodiments, the sequencing method includes enzymatic steps to amplify the SOMAmer sequence or to convert any kind of nucleic acid, including RNA and DNA that contain chemical modifications to any position, to any other kind of nucleic acid appropriate for sequencing.

In some embodiments, the sequencing method includes one or more cloning steps. In other embodiments the sequencing method includes a direct sequencing method without cloning.

In some embodiments, the sequencing method includes a directed approach with specific primers that target one or more SOMAmers in the test sample. In other embodiments, the sequencing method includes a shotgun approach that targets all SOMAmers in the test sample.

In some embodiments, the sequencing method includes enzymatic steps to amplify the molecule targeted for sequencing. In other embodiments, the sequencing method directly sequences single molecules. An exemplary nucleic acid sequencing-based method that can be used to detect a biomarker value corresponding to a biomarker in a biological sample includes the following: (a) converting a mixture of SOMAmers that contain chemically modified nucleotides to unmodified nucleic acids with an enzymatic step; (b) shotgun sequencing the resulting unmodified nucleic acids with a massively parallel sequencing platform such as, for example, the 454 Sequencing System (454 Life Sciences/Roche), the Illumina Sequencing System (Illumina), the ABI SOLiD Sequencing System (Applied Biosystems), the HeliScope Single Molecule Sequencer (Helicos Biosciences), or the Pacific Biosciences Real Time Single-Molecule Sequencing System (Pacific BioSciences) or the Polonator G Sequencing System (Dover Systems); and (c) identifying and quantifying the SOMAmers present in the mixture by specific sequence and sequence count.

### Determination of Biomarker Values Using Immunoassays

Immunoassay methods are based on the reaction of an antibody to its corresponding target or analyte and can detect the analyte in a sample depending on the specific assay format. To improve specificity and sensitivity of an assay method based on immuno-reactivity, monoclonal antibodies are often used because of their specific epitope recognition. Polyclonal antibodies have also been successfully used in various immunoassays because of their increased affinity for the target as compared to monoclonal antibodies. Immunoassays have been designed for use with a wide range of biological sample matrices. Immunoassay formats have been designed to provide qualitative, semi-quantitative, and quantitative results.

Quantitative results are generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I125) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Determination of Biomarker Values Using Gene Expression Profiling

Measuring mRNA in a biological sample may be used as a surrogate for detection of the level of the corresponding protein in the biological sample. Thus, any of the biomarkers or biomarker panels described herein can also be detected by detecting the appropriate RNA.

mRNA expression levels are measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

miRNA molecules are small RNAs that are non-coding but may regulate gene expression. Any of the methods suited to the measurement of mRNA expression levels can also be used for the corresponding miRNA. Recently many laboratories have investigated the use of miRNAs as biomarkers for disease. Many diseases involve wide-spread transcriptional regulation, and it is not surprising that miRNAs might find a role as biomarkers. The connection between miRNA concentrations and disease is often even less clear than the connections between protein levels and disease, yet the value of miRNA biomarkers might be substantial. Of course, as with any RNA expressed differentially during disease, the problems facing the development of an in vitro diagnostic product will include the requirement that the miRNAs survive in the diseased cell and are easily extracted for analysis, or that the miRNAs are released into blood or other matrices where they must survive long enough to be measured. Protein biomarkers have similar requirements, although many potential protein biomarkers are secreted intentionally at the site of pathology and function, during disease, in a paracrine fashion. Many potential protein biomarkers are designed to function outside the cells within which those proteins are synthesized.

### Detection of Biomarkers Using In Vivo Molecular Imaging Technologies

Any of the described biomarkers (see Table 8) may also be used in molecular imaging tests. For example, an imaging agent can be coupled to any of the described biomarkers, which can be used to aid in prediction of risk of renal insufficiency within 4 years, to monitor response to therapeutic interventions, to select a population for clinical trials among other uses.

In vivo imaging technologies provide non-invasive methods for determining the state of a particular disease or condition in the body of an individual. For example, entire portions of the body, or even the entire body, may be viewed as a three dimensional image, thereby providing valuable information concerning morphology and structures in the body. Such technologies may be combined with the detection of the biomarkers described herein to provide information concerning the renal health status of an individual.

The use of in vivo molecular imaging technologies is expanding due to various advances in technology. These advances include the development of new contrast agents or labels, such as radiolabels and/or fluorescent labels, which can provide strong signals within the body; and the development of powerful new imaging technology, which can detect and analyze these signals from outside the body, with sufficient sensitivity and accuracy to provide useful information. The contrast agent can be visualized in an appropriate imaging system, thereby providing an image of the portion or portions of the body in which the contrast agent is located. The contrast agent may be bound to or associated with a capture reagent, such as a SOMAmer or an antibody, for example, and/or with a peptide or protein, or an oligonucleotide (for example, for the detection of gene expression), or a complex containing any of these with one or more macromolecules and/or other particulate forms.

The contrast agent may also feature a radioactive atom that is useful in imaging. Suitable radioactive atoms include technetium-99m or iodine-123 for scintigraphic studies. Other readily detectable moieties include, for example, spin labels for magnetic resonance imaging (MRI) such as, for example, iodine-123 again, iodine-131, indium-111, fluorine-19, carbon-13, nitrogen-15, oxygen-17, gadolinium, manganese or iron. Such labels are well known in the art and could easily be selected by one of ordinary skill in the art.

Standard imaging techniques include but are not limited to magnetic resonance imaging, computed tomography scanning (coronary calcium score), positron emission tomography (PET), single photon emission computed tomography (SPECT), computed tomography angiography, and the like. For diagnostic in vivo imaging, the type of detection instrument available is a major factor in selecting a given contrast agent, such as a given radionuclide and the particular biomarker that it is used to target (protein, mRNA, and the like). The radionuclide chosen typically has a type of decay that is detectable by a given type of instrument. Also, when selecting a radionuclide for in vivo diagnosis, its half-life should be long enough to enable detection at the time of maximum uptake by the target tissue but short enough that deleterious radiation of the host is minimized.

Exemplary imaging techniques include but are not limited to PET and SPECT, which are imaging techniques in which a radionuclide is synthetically or locally administered to an individual. The subsequent uptake of the radiotracer is measured over time and used to obtain information about the targeted tissue and the biomarker. Because of the high-energy (gamma-ray) emissions of the specific isotopes employed and the sensitivity and sophistication of the instruments used to detect them, the two-dimensional distribution of radioactivity may be inferred from outside of the body.

Commonly used positron-emitting nuclides in PET include, for example, carbon-11, nitrogen-13, oxygen-15, and fluorine-18. Isotopes that decay by electron capture and/or gamma-emission are used in SPECT and include, for example iodine-123 and technetium-99m. An exemplary method for labeling amino acids with technetium-99m is the reduction of pertechnetate ion in the presence of a chelating precursor to form the labile technetium-99m-precursor complex, which, in turn, reacts with the metal binding group of a bifunctionally modified chemotactic peptide to form a technetium-99m-chemotactic peptide conjugate.

Antibodies are frequently used for such in vivo imaging diagnostic methods. The preparation and use of antibodies for in vivo diagnosis is well known in the art. Labeled antibodies which specifically bind any of the biomarkers in Table 8 can be injected into an individual suspected of having an increased risk of renal insufficiency, detectable according to the particular biomarker used, for the purpose of diagnosing or evaluating the disease status or condition of the individual. The label used will be selected in accordance with the imaging modality to be used, as previously described. Localization of the label permits determination of the tissue damage or other indications related to the risk of renal insufficiency. The amount of label within an organ or tissue also allows determination of the involvement of the renal insufficiency biomarkers due to the risk of renal insufficiency in that organ or tissue.

Similarly, SOMAmers may be used for such in vivo imaging diagnostic methods. For example, a SOMAmer that was used to identify a particular biomarker described in Table 8 (and therefore binds specifically to that particular biomarker) may be appropriately labeled and injected into an individual being evaluated for renal insufficiency, detectable according to the particular biomarker, for the purpose of diagnosing or evaluating the levels of tissue damage, atherosclerotic plaques, components of inflammatory response and other factors associated with the risk of renal insufficiency in the individual. The label used will be selected in accordance with the imaging modality to be used, as previously described. Localization of the label permits determination of the site of the processes leading to increased risk. The amount of label within an organ or tissue also allows determination of the infiltration of the pathological process in that organ or tissue. SOMAmer-directed imaging agents could have unique and advantageous characteristics relating to tissue penetration, tissue distribution, kinetics, elimination, potency, and selectivity as compared to other imaging agents.

Such techniques may also optionally be performed with labeled oligonucleotides, for example, for detection of gene expression through imaging with antisense oligonucleotides. These methods are used for in situ hybridization, for example, with fluorescent molecules or radionuclides as the label. Other methods for detection of gene expression include, for example, detection of the activity of a reporter gene.

Another general type of imaging technology is optical imaging, in which fluorescent signals within the subject are detected by an optical device that is external to the subject. These signals may be due to actual fluorescence and/or to bioluminescence. Improvements in the sensitivity of optical detection devices have increased the usefulness of optical imaging for in vivo diagnostic assays.

The use of in vivo molecular biomarker imaging is increasing, including for clinical trials, for example, to more rapidly measure clinical efficacy in trials for new disease or condition therapies and/or to avoid prolonged treatment with a placebo for those diseases, such as multiple sclerosis, in which such prolonged treatment may be considered to be ethically questionable.

For a review of other techniques, see N. Blow, Nature Methods, 6, 465-469, 2009.

### Determination of Biomarker Values Using Mass Spectrometry Methods

A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al. Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS)N, atmospheric pressure photoionization mass spectrometry (APPI-MS), APPI-MS/MS, and APPI-(MS)N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to SOMAmers, antibodies, nucleic acid probes, chimeras, small molecules, an F(ab′)2 fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Determination of Biomarker Values Using a Proximity Ligation Assay

A proximity ligation assay can be used to determine biomarker values. Briefly, a test sample is contacted with a pair of affinity probes that may be a pair of antibodies or a pair of SOMAmers, with each member of the pair extended with an oligonucleotide. The targets for the pair of affinity probes may be two distinct determinates on one protein or one determinate on each of two different proteins, which may exist as homo- or hetero-multimeric complexes. When probes bind to the target determinates, the free ends of the oligonucleotide extensions are brought into sufficiently close proximity to hybridize together. The hybridization of the oligonucleotide extensions is facilitated by a common connector oligonucleotide which serves to bridge together the oligonucleotide extensions when they are positioned in sufficient proximity. Once the oligonucleotide extensions of the probes are hybridized, the ends of the extensions are joined together by enzymatic DNA ligation.

Each oligonucleotide extension comprises a primer site for PCR amplification. Once the oligonucleotide extensions are ligated together, the oligonucleotides form a continuous DNA sequence which, through PCR amplification, reveals information regarding the identity and amount of the target protein, as well as, information regarding protein-protein interactions where the target determinates are on two different proteins. Proximity ligation can provide a highly sensitive and specific assay for real-time protein concentration and interaction information through use of real-time PCR. Probes that do not bind the determinates of interest do not have the corresponding oligonucleotide extensions brought into proximity and no ligation or PCR amplification can proceed, resulting in no signal being produced.

The foregoing assays enable the detection of biomarker values that are useful in methods for prediction of renal insufficiency, where the methods comprise detecting, in a biological sample from an individual, biomarker values that each correspond to a biomarker selected from the group consisting of the biomarkers provided in Table 8, wherein a classification, as described in detail below, using the biomarker values indicates whether the individual has elevated risk of developing renal insufficiency within a 4 year time period. While certain of the described renal insufficiency biomarkers are useful alone for predicting risk of renal insufficiency, methods are also described herein for the grouping of multiple subsets of the renal insufficiency biomarkers that are each useful as a panel of three or more biomarkers. In accordance with any of the methods described herein, biomarker values can be detected and classified individually or they can be detected and classified collectively, as for example in a multiplex assay format.

A biomarker “signature” for a given diagnostic or predictive test contains a set of markers, each marker having different levels in the populations of interest. Different levels, in this context, may refer to different means of the marker levels for the individuals in two or more groups, or different variances in the two or more groups, or a combination of both. For the simplest form of a diagnostic test, these markers can be used to assign an unknown sample from an individual into one of two groups, either at increased risk of renal insufficiency or not. The assignment of a sample into one of two or more groups is known as classification, and the procedure used to accomplish this assignment is known as a classifier or a classification method. Classification methods may also be referred to as scoring methods. There are many classification methods that can be used to construct a diagnostic classifier from a set of biomarker values. In general, classification methods are most easily performed using supervised learning techniques where a data set is collected using samples obtained from individuals within two (or more, for multiple classification states) distinct groups one wishes to distinguish. Since the class (group or population) to which each sample belongs is known in advance for each sample, the classification method can be trained to give the desired classification response. It is also possible to use unsupervised learning techniques to produce a diagnostic classifier.

Common approaches for developing diagnostic classifiers include decision trees; bagging, boosting, forests and random forests; rule inference based learning; Parzen Windows; linear models; logistic; neural network methods; unsupervised clustering; K-means; hierarchical ascending/descending; semi-supervised learning; prototype methods; nearest neighbor; kernel density estimation; support vector machines; hidden Markov models; Boltzmann Learning; and classifiers may be combined either simply or in ways which minimize particular objective functions. For a review, see, e.g., Pattern Classification, R. O. Duda, et al., editors, John Wiley & Sons, 2nd edition, 2001; see also, The Elements of Statistical Learning—Data Mining, Inference, and Prediction, T. Hastie, et al., editors, Springer Science+Business Media, LLC, 2nd edition, 2009; each of which is incorporated by reference in its entirety.

To produce a classifier using supervised learning techniques, a set of samples called training data are obtained. In the context of diagnostic tests, training data includes samples from the distinct groups (classes) to which unknown samples will later be assigned. For example, samples collected from individuals in a control population and individuals in a particular disease, condition or event population can constitute training data to develop a classifier that can classify unknown samples (or, more particularly, the individuals from whom the samples were obtained) as either having the disease, condition or elevated risk of an event or being free from the disease, condition or elevated risk of an event. The development of the classifier from the training data is known as training the classifier. Specific details on classifier training depend on the nature of the supervised learning technique (see, e.g., Pattern Classification, R. O. Duda, et al., editors, John Wiley & Sons, 2nd edition, 2001; see also, The Elements of Statistical Learning—Data Mining, Inference, and Prediction, T. Hastie, et al., editors, Springer Science+Business Media, LLC, 2nd edition, 2009).

Since typically there are many more potential biomarker values than samples in a training set, care must be used to avoid over-fitting. Over-fitting occurs when a statistical model describes random error or noise instead of the underlying relationship. Over-fitting can be avoided in a variety of ways, including, for example, by limiting the number of markers used in developing the classifier, by assuming that the marker responses are independent of one another, by limiting the complexity of the underlying statistical model employed, and by ensuring that the underlying statistical model conforms to the data.

In order to identify a set of biomarkers associated with occurrence of events, the combined set of control and early event samples were analyzed using Principal Component Analysis (PCA). PCA displays the samples with respect to the axes defined by the strongest variations between all the samples, without regard to the case or control outcome, thus mitigating the risk of overfitting the distinction between case and control. Since the occurrence of serious thrombotic events has a strong component of chance involved, requiring unstable plaque to rupture in vital vessels to be reported, one would not expect to see a clear separation between the control and event sample sets. While the observed separation between case and control is not large, it occurs on the second principal component, corresponding to around 10% of the total variation in this set of samples, which indicates that the underlying biological variation is relatively simple to quantify.

In the next set of analyses, biomarkers can be analyzed for those components of difference between samples which were specific to the separation between the control samples and early event samples. One method that may be employed is the use of DSGA (Bair, E. and Tibshirani, R. (2004) Semi-supervised methods to predict patient survival from gene expression data. PLOS Biol., 2, 511-522) to remove (deflate) the first three principal component directions of variation between the samples in the control set. Although the dimensionality reduction is performed on the control set to discover, both the samples in the control and the samples from the early event samples are run through the PCA. Separation of cases from early events can be observed along the horizontal axis.

### Cross Validated Selection of Proteins Relevant to Renal Insufficiency

In order to avoid over-fitting of protein predictive power to idiosyncratic features of a particular selection of samples, a cross-validation and dimensional reduction approach can be taken. Cross-validation involves the multiple selection of sets of samples to determine the association of risk by protein combined with the use of the unselected samples to monitor the ability of the method to apply to samples which were not used in producing the model of risk (The Elements of Statistical Learning—Data Mining, Inference, and Prediction, T. Hastie, et al., editors, Springer Science+Business Media, LLC, 2nd edition, 2009). We applied the supervised PCA method of Tibshirani et al (Bair, E. and Tibshirani, R. (2004) Semi-supervised methods to predict patient survival from gene expression data. PLOS Biol., 2, 511-522.) which is applicable to high dimensional datasets in the modeling of risk of renal insufficiency. The supervised PCA (SPCA) method involves the univariate selection of a set of proteins statistically associated with the observed event hazard in the data and the determination of the correlated component which combines information from all of these proteins. This determination of the correlated component is a dimensionality reduction step which not only combines information across proteins, but also mitigates the likelihood of overfitting by reducing the number of independent variables from the full protein menu of over 1000 proteins down to a few principal components (in this work, we only examined the first principal component).

### Univariate Analysis and Multivariate Analysis of the Relationship of Individual Proteins to Time to Event

The Cox proportional hazard model (Cox, David R (1972). “Regression Models and Life-Tables”. Journal of the Royal Statistical Society. Series B (Methodological) 34 (2): 187-220.)) is widely used in medical statistics. Cox regression avoids fitting a specific function of time to the cumulative survival, and instead employs a model of relative risk referred to a baseline hazard function (which may vary with time). The baseline hazard function describes the common shape of the survival time distribution for all individuals, while the relative risk gives the level of the hazard for a set of covariate values (such as a single individual or group), as a multiple of the baseline hazard. The relative risk is constant with time in the Cox model.

### Kits

Any combination of the biomarkers of Table 8 can be detected using a suitable kit, such as for use in performing the methods disclosed herein. Furthermore, any kit can contain one or more detectable labels as described herein, such as a fluorescent moiety, etc.

In one embodiment, a kit includes (a) one or more capture reagents (such as, for example, at least one SOMAmer or antibody) for detecting one or more biomarkers in a biological sample, wherein the biomarkers include any of the biomarkers set forth in Table 8 and optionally (b) one or more software or computer program products for classifying the individual from whom the biological sample was obtained as either having or not having increased risk of renal insufficiency or for determining the likelihood that the individual has increased risk of renal insufficiency, as further described herein. Alternatively, rather than one or more computer program products, one or more instructions for manually performing the above steps by a human can be provided.

The combination of a solid support with a corresponding capture reagent having a signal generating material is referred to herein as a “detection device” or “kit”. The kit can also include instructions for using the devices and reagents, handling the sample, and analyzing the data. Further the kit may be used with a computer system or software to analyze and report the result of the analysis of the biological sample.

The kits can also contain one or more reagents (e.g., solubilization buffers, detergents, washes, or buffers) for processing a biological sample. Any of the kits described herein can also include, e.g., buffers, blocking agents, mass spectrometry matrix materials, antibody capture agents, positive control samples, negative control samples, software and information such as protocols, guidance and reference data.

In one aspect, the invention provides kits for the analysis of renal insufficiency risk status. The kits include PCR primers for one or more SOMAmers specific to biomarkers selected from Table 8. The kit may further include instructions for use and correlation of the biomarkers with prediction of risk of renal insufficiency risk. The kit may also include a DNA array containing the complement of one or more of the Somamers specific for the biomarkers selected from Table 8, reagents, and/or enzymes for amplifying or isolating sample DNA. The kits may include reagents for real-time PCR, for example, TaqMan probes and/or primers, and enzymes.

For example, a kit can comprise (a) reagents comprising at least capture reagent for quantifying one or more biomarkers in a test sample, wherein said biomarkers comprise the biomarkers set forth in Table 8, or any other biomarkers or biomarkers panels described herein, and optionally (b) one or more algorithms or computer programs for performing the steps of comparing the amount of each biomarker quantified in the test sample to one or more predetermined cutoffs and assigning a score for each biomarker quantified based on said comparison, combining the assigned scores for each biomarker quantified to obtain a total score, comparing the total score with a predetermined score, and using said comparison to determine whether an individual has an increased risk of renal insufficiency. Alternatively, rather than one or more algorithms or computer programs, one or more instructions for manually performing the above steps by a human can be provided.

### Computer Methods and Software

Once a biomarker or biomarker panel is selected, a method for diagnosing an individual can comprise the following: 1) collect or otherwise obtain a biological sample; 2) perform an analytical method to detect and measure the biomarker or biomarkers in the panel in the biological sample; 3) perform any data normalization or standardization required for the method used to collect biomarker values; 4) calculate the marker score; 5) combine the marker scores to obtain a total diagnostic or predictive score; and 6) report the individual's diagnostic or predictive score. In this approach, the diagnostic or predictive score may be a single number determined from the sum of all the marker calculations that is compared to a preset threshold value that is an indication of the presence or absence of disease. Or the diagnostic or predictive score may be a series of bars that each represent a biomarker value and the pattern of the responses may be compared to a pre-set pattern for determination of the presence or absence of disease, condition or the increased risk (or not) of an event.

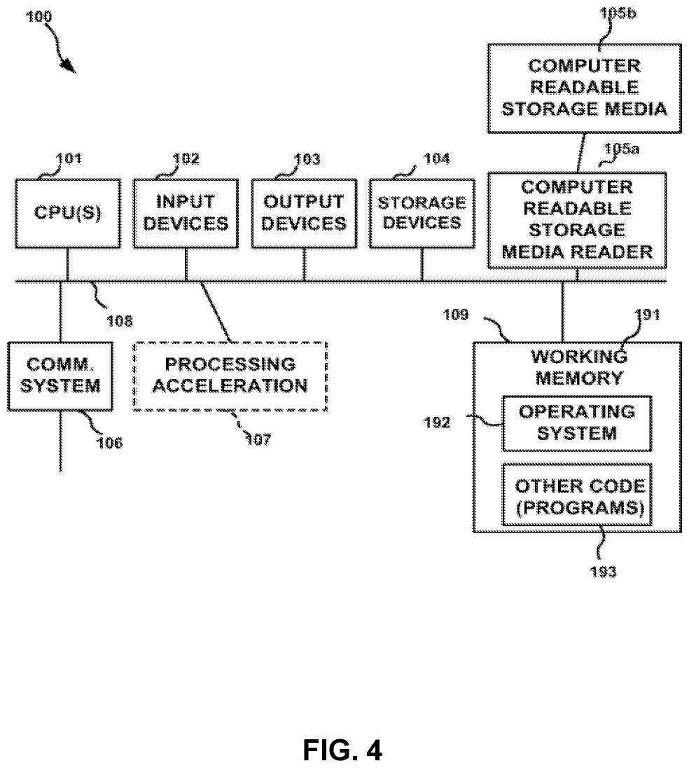

At least some embodiments of the methods described herein can be implemented with the use of a computer. An example of a computer system 100 is shown in FIG. 4. With reference to FIG. 4, system 100 is shown comprised of hardware elements that are electrically coupled via bus 108, including a processor 101, input device 102, output device 103, storage device 104, computer-readable storage media reader 105a, communications system 106, processing acceleration (e.g., DSP or special-purpose processors) 107 and memory 109. Computer-readable storage media reader 105a is further coupled to computer-readable storage media 105b, the combination comprehensively representing remote, local, fixed and/or removable storage devices plus storage media, memory, etc. for temporarily and/or more permanently containing computer-readable information, which can include storage device 104, memory 109 and/or any other such accessible system 100 resource. System 100 also comprises software elements (shown as being currently located within working memory 191) including an operating system 192 and other code 193, such as programs, data and the like.

With respect to FIG. 4, system 100 has extensive flexibility and configurability. Thus, for example, a single architecture might be utilized to implement one or more servers that can be further configured in accordance with currently desirable protocols, protocol variations, extensions, etc. However, it will be apparent to those skilled in the art that embodiments may well be utilized in accordance with more specific application requirements. For example, one or more system elements might be implemented as sub-elements within a system 100 component (e.g., within communications system 106). Customized hardware might also be utilized and/or particular elements might be implemented in hardware, software or both. Further, while connection to other computing devices such as network input/output devices (not shown) may be employed, it is to be understood that wired, wireless, modem, and/or other connection or connections to other computing devices might also be utilized.

In one aspect, the system can comprise a database containing features of biomarkers characteristic of prediction of risk of renal insufficiency. The biomarker data (or biomarker information) can be utilized as an input to the computer for use as part of a computer implemented method. The biomarker data can include the data as described herein.

In one aspect, the system further comprises one or more devices for providing input data to the one or more processors.

The system further comprises a memory for storing a data set of ranked data elements.

In another aspect, the device for providing input data comprises a detector for detecting the characteristic of the data element, e.g., such as a mass spectrometer or gene chip reader.

The system additionally may comprise a database management system. User requests or queries can be formatted in an appropriate language understood by the database management system that processes the query to extract the relevant information from the database of training sets.

The system may be connectable to a network to which a network server and one or more clients are connected. The network may be a local area network (LAN) or a wide area network (WAN), as is known in the art. Preferably, the server includes the hardware necessary for running computer program products (e.g., software) to access database data for processing user requests.

The system may include an operating system (e.g., UNIX or Linux) for executing instructions from a database management system. In one aspect, the operating system can operate on a global communications network, such as the internet, and utilize a global communications network server to connect to such a network.

The system may include one or more devices that comprise a graphical display interface comprising interface elements such as buttons, pull down menus, scroll bars, fields for entering text, and the like as are routinely found in graphical user interfaces known in the art. Requests entered on a user interface can be transmitted to an application program in the system for formatting to search for relevant information in one or more of the system databases. Requests or queries entered by a user may be constructed in any suitable database language.

The graphical user interface may be generated by a graphical user interface code as part of the operating system and can be used to input data and/or to display inputted data. The result of processed data can be displayed in the interface, printed on a printer in communication with the system, saved in a memory device, and/or transmitted over the network or can be provided in the form of the computer readable medium.

The system can be in communication with an input device for providing data regarding data elements to the system (e.g., expression values). In one aspect, the input device can include a gene expression profiling system including, e.g., a mass spectrometer, gene chip or array reader, and the like.

The methods and apparatus for analyzing renal insufficiency risk prediction biomarker information according to various embodiments may be implemented in any suitable manner, for example, using a computer program operating on a computer system. A conventional computer system comprising a processor and a random access memory, such as a remotely-accessible application server, network server, personal computer or workstation may be used. Additional computer system components may include memory devices or information storage systems, such as a mass storage system and a user interface, for example a conventional monitor, keyboard and tracking device. The computer system may be a stand-alone system or part of a network of computers including a server and one or more databases.

The renal insufficiency risk prediction biomarker analysis system can provide functions and operations to complete data analysis, such as data gathering, processing, analysis, reporting and/or diagnosis. For example, in one embodiment, the computer system can execute the computer program that may receive, store, search, analyze, and report information relating to the renal insufficiency risk prediction biomarkers. The computer program may comprise multiple modules performing various functions or operations, such as a processing module for processing raw data and generating supplemental data and an analysis module for analyzing raw data and supplemental data to generate a renal insufficiency risk prediction status and/or diagnosis or risk calculation. Calculation of risk status for renal insufficiency may optionally comprise generating or collecting any other information, including additional biomedical information, regarding the condition of the individual relative to the disease, condition or event, identifying whether further tests may be desirable, or otherwise evaluating the health status of the individual.

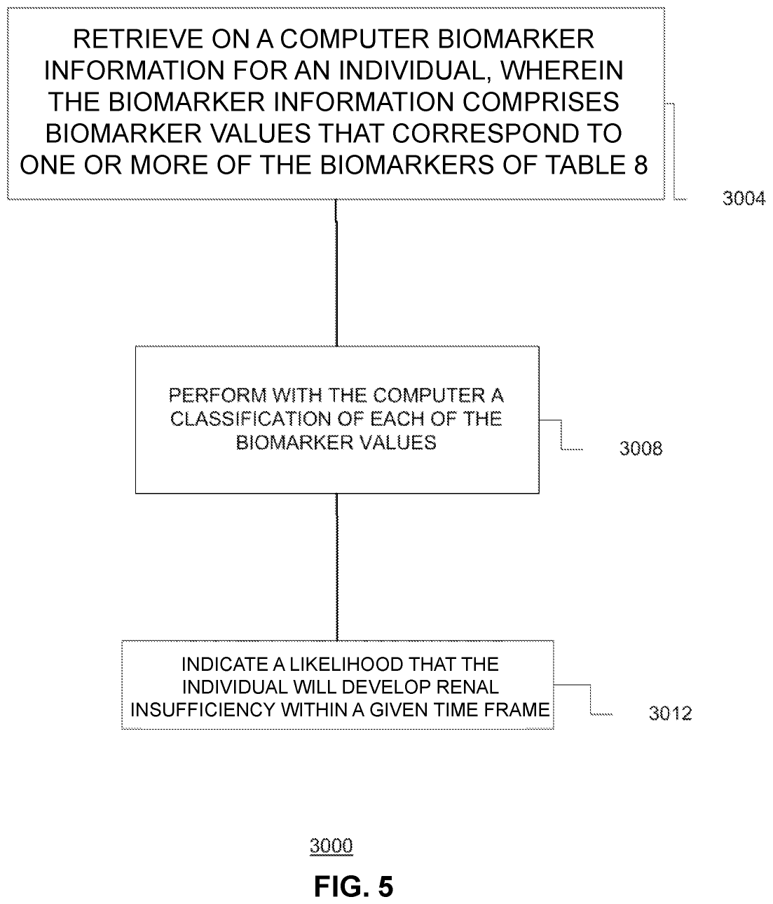

Referring now to FIG. 5, an example of a method of utilizing a computer in accordance with principles of a disclosed embodiment can be seen. In FIG. 5, a flowchart 3000 is shown. In block 3004, biomarker information can be retrieved for an individual. The biomarker information can be retrieved from a computer database, for example, after testing of the individual's biological sample is performed. The biomarker information can comprise biomarker values that each correspond to one or more of the biomarkers of Table 8. In block 3008, a computer can be utilized to classify each of the biomarker values. And, in block 3012, a determination can be made as to the likelihood that an individual has increased risk of renal insufficiency based upon a plurality of classifications. The indication can be output to a display or other indicating device so that it is viewable by a person. Thus, for example, it can be displayed on a display screen of a computer or other output device.

Some embodiments described herein can be implemented so as to include a computer program product. A computer program product may include a computer readable medium having computer readable program code embodied in the medium for causing an application program to execute on a computer with a database.

As used herein, a “computer program product” refers to an organized set of instructions in the form of natural or programming language statements that are contained on a physical media of any nature (e.g., written, electronic, magnetic, optical or otherwise) and that may be used with a computer or other automated data processing system. Such programming language statements, when executed by a computer or data processing system, cause the computer or data processing system to act in accordance with the particular content of the statements. Computer program products include without limitation: programs in source and object code and/or test or data libraries embedded in a computer readable medium. Furthermore, the computer program product that enables a computer system or data processing equipment device to act in pre-selected ways may be provided in a number of forms, including, but not limited to, original source code, assembly code, object code, machine language, encrypted or compressed versions of the foregoing and any and all equivalents.

In one aspect, a computer program product is provided for evaluation of the risk of renal insufficiency. The computer program product includes a computer readable medium embodying program code executable by a processor of a computing device or system, the program code comprising: code that retrieves data attributed to a biological sample from an individual, wherein the data comprises biomarker values that each correspond to one or more of the biomarkers of Table 8; and code that executes a classification method that indicates a renal insufficiency risk status of the individual as a function of the biomarker values.

In still another aspect, a computer program product is provided for indicating a likelihood of risk of renal insufficiency. The computer program product includes a computer readable medium embodying program code executable by a processor of a computing device or system, the program code comprising: code that retrieves data attributed to a biological sample from an individual, wherein the data comprises a biomarker value corresponding to one or more of the biomarkers of Table 8; and code that executes a classification method that indicates a renal insufficiency risk status of the individual as a function of the biomarker value.

While various embodiments have been described as methods or apparatuses, it should be understood that embodiments can be implemented through code coupled with a computer, e.g., code resident on a computer or accessible by the computer. For example, software and databases could be utilized to implement many of the methods discussed above. Thus, in addition to embodiments accomplished by hardware, it is also noted that these embodiments can be accomplished through the use of an article of manufacture comprised of a computer usable medium having a computer readable program code embodied therein, which causes the enablement of the functions disclosed in this description. Therefore, it is desired that embodiments also be considered protected by this patent in their program code means as well. Furthermore, the embodiments may be embodied as code stored in a computer-readable memory of virtually any kind including, without limitation, RAM, ROM, magnetic media, optical media, or magneto-optical media. Even more generally, the embodiments could be implemented in software, or in hardware, or any combination thereof including, but not limited to, software running on a general purpose processor, microcode, PLAs, or ASICs.

It is also envisioned that embodiments could be accomplished as computer signals embodied in a carrier wave, as well as signals (e.g., electrical and optical) propagated through a transmission medium. Thus, the various types of information discussed above could be formatted in a structure, such as a data structure, and transmitted as an electrical signal through a transmission medium or stored on a computer readable medium.

It is also noted that many of the structures, materials, and acts recited herein can be recited as means for performing a function or step for performing a function. Therefore, it should be understood that such language is entitled to cover all such structures, materials, or acts disclosed within this specification and their equivalents, including the matter incorporated by reference.

The biomarker identification process, the utilization of the biomarkers disclosed herein, and the various methods for determining biomarker values are described in detail above with respect to evaluation of risk of a renal insufficiency. However, the application of the process, the use of identified biomarkers, and the methods for determining biomarker values are fully applicable to other specific types of diseases or medical conditions, or to the identification of individuals who may or may not be benefited by an ancillary medical treatment.

### EXAMPLES

The following examples are provided for illustrative purposes only and are not intended to limit the scope of the application as defined by the appended claims. All examples described herein were carried out using standard techniques, which are well known and routine to those of skill in the art. Routine molecular biology techniques described in the following examples can be carried out as described in standard laboratory manuals, such as Sambrook et al., Molecular Cloning: A Laboratory Manual, 3rd. ed., Cold Spring Harbor Laboratory Press, Cold Spring Harbor, N.Y., (2001).

### Example 1. Model Specification

1.1 Endpoint description. Development of PCRI (as a yes or no binary status) within four years of the blood sample. PCRI is defined as at least one of the following events: (1) a 50% decline in estimated glomerular filtration rate (eGFR), (2) a diagnosis that kidney dialysis is needed, (3) development of eGFR<15 ml/min/1.73 m2, (4) development of end stage renal disease (ESRD), or (5) a diagnosis that a kidney transplantation is needed.
1.2 Model Information. The model is a logistic regression model with 10 features with non-zero coefficients. The model was trained on PCRI within four years as the endpoint. The model provides two predictions. (1) PCRI by four years as a yes/no binary variable: In model development, an optimal probability score of 0.3533 was identified as the threshold to classify status as “yes” vs “no” for PCRI within the next four years. (2) The relative risk: This is a continuous value that allows for the model prediction to be interpreted such that higher predictions indicate a greater likelihood of developing PCRI within the next four years. The probability that an individual develops PCRI is generated by the model and is represented by p*. The probability is used to calculate the relative risk,

$$R R = \frac{p *}{q}$$

with p equal to the probability as defined above, and q equal to the probability for the baseline individual in the training cohort. A “baseline” individual is defined as an individual with model feature values set to zero in the training set. Such individual may be referred to as an “average individual” herein and in the model interpretation. By construction, the analytes for a baseline individual are set to zero because the RFU measurements are centered around the mean. For this model, q is equal to 0.309, which was calculated using the training cohort. The reference population ranges from CKD stages 1 to 5 with 80 of the population having mild to severe chronic kidney disease (stages 3-5) and a mean eGFR of 43 ml/min/1.73 m2. The relative risks are then sorted into quintiles based on the relative risks for the full training data set (Table 7). The relative risks and the quintile bins as defined by the training data.

TABLE 6
Scoring rules

| Test Outcome X (probability) | Predicted Class |
|---|---|
| X < 0.3533 | No PCRI within 4 years |
| X ≥ 0.3533 | Yes PCRI within 4 years |

TABLE 7
Scoring rules for PCRI rates and RR's in
each quintile, derived from the training data

| Quintile | PCRI rate (95% CI) | median RR | RR range |
|---|---|---|---|
| 1 | 6.5% (4.8%-8.4%) | 0.216 | [0., 0.36] |
| 2 | 15.6% (12.7%-18.4%) | 0.526 | (0.36, 0.7] |
| 3 (baseline) | 27.4% (24.2%-30.9%) | 0.930 | (0.7, 1.21] |
| 4 | 51.6% (47.9%-55.5%) | 1.57 | (1.21, 1.95] |
| 5 | 76.2% (73.0%-79.5%) | 2.50 | (1.95, 3.24] |

TABLE 8
Features included in final model

| | | Importance |
|---|---|---|
| Target | Target Full Name | (Coefficient) |
| HAVCR1 | Hepatitis A virus cellular receptor 1 | 0.6968902 |
| FSTL3 | Follistatin-related protein 3 | 0.4605888 |
| RGMB | RGM domain family member B | 0.3277384 |
| COL28A1 | Collagen alpha-1(XXVIII) chain | 0.5342980 |
| UBE2G2 | Ubiquitin-conjugating enzyme E2 G2 | 0.4338253 |
| REG1A | Lithostathine-1-alpha | −0.3986397 |
| REG1B | Lithostathine-1-beta | 0.2507715 |
| COL6A3 | Collagen alpha-3(VI) chain | −1.1526061 |
| CST3 | Cystatin-C | 0.0884266 |
| TNFRSF1A | Tumor necrosis factor receptor | 0.5305810 |
| | superfamily member 1A | |

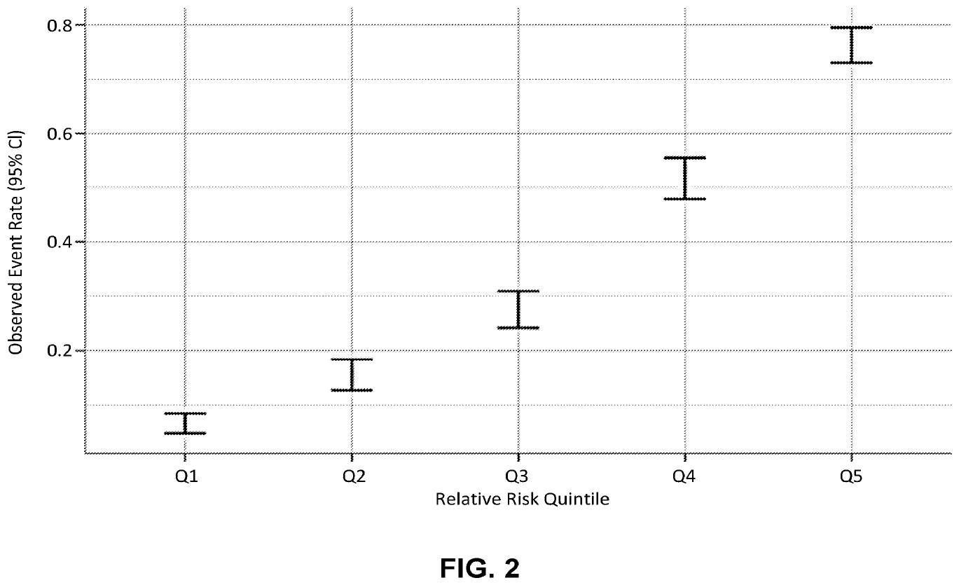

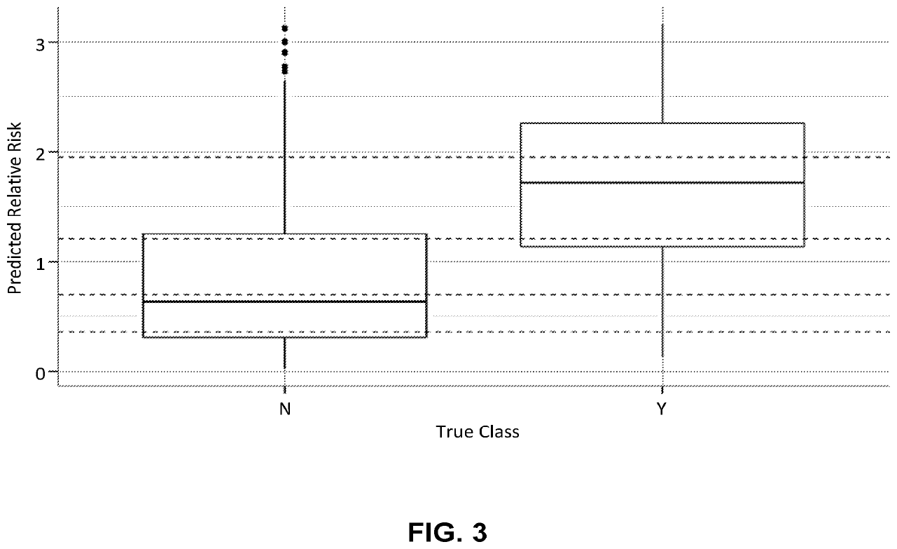

### 1.3 Reporting Business Rules

| Usage | Rule | Rationale |
|---|---|---|
| LDT | PCRI risk is reported as a | This information will help |
| | relative risk compared to | guide clinical decision |
| | average in CRIC training data | making by providing health |
| | set, which included participants | care providers and patients |
| | from CKD Stages 1-5. | with a tool to understand |
| | Possible values for relative risk | the risk for PCRI compared |
| | are 0.01 to 3.24 and may be | to other people with a |
| | converted to percent higher/ | similar disease process. |
| | lower than the reference | |
| | population average risk | |
| | PCRI risk can also be reported in | Providing quintiles provides |
| | quintiles based on the reference | a simple interpretation of |
| | population (FIGS. 2 and 3). | PCRI risk compared to |
| | | other people with a |
| | | similar disease process. |
| RUO | PCRI risk will be reported as the | Reporting the precise |
| | probability up to four decimal | probability of PCRI is |
| | places binary (yes/no) class. | necessary for research use. |

1.4 Hypothetical patient. A hypothetical patient has a predicted probability equal to 0.72 based on the proteomic model. This patient's relative risk is 2.33 and thus their bin is quintile four.

$$R R = \frac{0.72}{0.309} = 2 \text{.33}$$

This relative risk in the example is interpreted as follows: this patient has 2.33 times higher risk alternatively, this patient has a 133% higher risk of developing PCRI compared to the average individual in our reference population (Table 7).

Further AUC values are provided in Tables 9a-9f for selected Table 8 features and combinations of Table 8 features.

TABLE 9a

| X1 | X2 | X3 | X4 | X5 | X6 | X7 | AUC |
|---|---|---|---|---|---|---|---|
| COL28A1 | | | | | | | 0.71264881 |
| UBE2G2 | | | | | | | 0.68253118 |
| REG1B | | | | | | | 0.67968056 |

TABLE 9b

| X1 | X2 | X3 | X4 | X5 | X6 | X7 | AUC |
|---|---|---|---|---|---|---|---|
| COL28A1 | UBE2G2 | | | | | | 0.7339549 |
| UBE2G2 | REG1B | | | | | | 0.72219532 |
| REG1B | COL28A1 | | | | | | 0.72218961 |
| COL28A1 | HAVCR1 | | | | | | 0.782208 |
| COL28A1 | FSTL3 | | | | | | 0.75680799 |
| COL28A1 | RGMB | | | | | | 0.74478181 |
| COL28A1 | REG1A | | | | | | 0.72673071 |
| COL28A1 | COL6A3 | | | | | | 0.70373903 |
| COL28A1 | CST3 | | | | | | 0.72592196 |
| COL28A1 | TNFRSF1A | | | | | | 0.76441209 |
| UBE2G2 | HAVCR1 | | | | | | 0.78973058 |
| UBE2G2 | FSTL3 | | | | | | 0.76989974 |
| UBE2G2 | RGMB | | | | | | 0.74470028 |
| UBE2G2 | REG1A | | | | | | 0.72822349 |
| UBE2G2 | COL6A3 | | | | | | 0.72424982 |
| UBE2G2 | CST3 | | | | | | 0.7445731 |
| UBE2G2 | TNFRSF1A | | | | | | 0.78187211 |
| REG1B | HAVCR1 | | | | | | 0.77857757 |
| REG1B | FSTL3 | | | | | | 0.75921143 |
| REG1B | RGMB | | | | | | 0.72903551 |
| REG1B | REG1A | | | | | | 0.69319911 |
| REG1B | COL6A3 | | | | | | 0.71661636 |
| REG1B | CST3 | | | | | | 0.72903551 |
| REG1B | TNFRSF1A | | | | | | 0.76557713 |

TABLE 9c

| X1 | X2 | X3 | X4 | X5 | X6 | X7 | AUC |
|---|---|---|---|---|---|---|---|
| COL28A1 | UBE2G2 | REG1B | | | | | 0.74102908 |
| COL28A1 | UBE2G2 | HAVCR1 | | | | | 0.79645093 |
| COL28A1 | UBE2G2 | FSTL3 | | | | | 0.76779224 |
| COL28A1 | UBE2G2 | RGMB | | | | | 0.7577105 |
| COL28A1 | UBE2G2 | REG1A | | | | | 0.74350263 |
| COL28A1 | UBE2G2 | COL6A3 | | | | | 0.73528136 |
| COL28A1 | UBE2G2 | CST3 | | | | | 0.74399588 |
| COL28A1 | UBE2G2 | TNFRSF1A | | | | | 0.78256183 |
| UBE2G2 | REG1B | HAVCR1 | | | | | 0.79730371 |
| UBE2G2 | REG1B | FSTL3 | | | | | 0.77068893 |
| UBE2G2 | REG1B | RGMB | | | | | 0.75150215 |
| UBE2G2 | REG1B | REG1A | | | | | 0.72698753 |
| UBE2G2 | REG1B | COL6A3 | | | | | 0.73328148 |
| UBE2G2 | REG1B | CST3 | | | | | 0.74792145 |
| UBE2G2 | REG1B | TNFRSF1A | | | | | 0.78193325 |
| REG1B | COL28A1 | HAVCR1 | | | | | 0.78585964 |
| REG1B | COL28A1 | FSTL3 | | | | | 0.75822576 |
| REG1B | COL28A1 | RGMB | | | | | 0.74558241 |
| REG1B | COL28A1 | REG1A | | | | | 0.723459 |
| REG1B | COL28A1 | COL6A3 | | | | | 0.71576929 |
| REG1B | COL28A1 | CST3 | | | | | 0.72992742 |
| REG1B | COL28A1 | TNFRSF1A | | | | | 0.764642 |

TABLE 9d

| X1 | X2 | X3 | X4 | X5 | X6 | X7 | AUC |
|---|---|---|---|---|---|---|---|
| COL28A1 | UBE2G2 | REG1B | HAVCR1 | | | | 0.79907613 |
| COL28A1 | UBE2G2 | REG1B | FSTL3 | | | | 0.76890265 |
| COL28A1 | UBE2G2 | REG1B | RGMB | | | | 0.75840838 |
| COL28A1 | UBE2G2 | REG1B | REG1A | | | | 0.74123045 |
| COL28A1 | UBE2G2 | REG1B | COL6A3 | | | | 0.74270774 |
| COL28A1 | UBE2G2 | REG1B | CST3 | | | | 0.74674255 |
| COL28A1 | UBE2G2 | REG1B | TNFRSF1A | | | | 0.78264336 |

TABLE 9e

| X1 | X2 | X3 | X4 | X5 | X6 | X7 | AUC |
|---|---|---|---|---|---|---|---|
| COL28A1 | UBE2G2 | REG1B | HAVCR1 | FSTL3 | | | 0.80978318 |
| COL28A1 | UBE2G2 | REG1B | HAVCR1 | RGMB | | | 0.81160126 |
| COL28A1 | UBE2G2 | REG1B | HAVCR1 | REG1A | | | 0.79808393 |
| COL28A1 | UBE2G2 | REG1B | HAVCR1 | COL6A3 | | | 0.79741459 |
| COL28A1 | UBE2G2 | REG1B | HAVCR1 | CST3 | | | 0.802815 |
| COL28A1 | UBE2G2 | REG1B | HAVCR1 | TNFRSF1A | | | 0.81340058 |
| COL28A1 | UBE2G2 | REG1B | FSTL3 | REG1A | | | 0.76963966 |
| COL28A1 | UBE2G2 | REG1B | FSTL3 | COL6A3 | | | 0.77194201 |
| COL28A1 | UBE2G2 | REG1B | FSTL3 | CST3 | | | 0.77019568 |
| COL28A1 | UBE2G2 | REG1B | FSTL3 | TNFRSF1A | | | 0.78897808 |
| COL28A1 | UBE2G2 | REG1B | RGMB | REG1A | | | 0.7573314 |
| COL28A1 | UBE2G2 | REG1B | RGMB | COL6A3 | | | 0.76121294 |
| COL28A1 | UBE2G2 | REG1B | RGMB | CST3 | | | 0.76114446 |
| COL28A1 | UBE2G2 | REG1B | RGMB | TNFRSF1A | | | 0.78617597 |
| COL28A1 | UBE2G2 | REG1B | REG1A | COL6A3 | | | 0.7438157 |
| COL28A1 | UBE2G2 | REG1B | REG1A | CST3 | | | 0.74585961 |
| COL28A1 | UBE2G2 | REG1B | REG1A | TNFRSF1A | | | 0.78220637 |
| COL28A1 | UBE2G2 | REG1B | COL6A3 | CST3 | | | 0.74900332 |
| COL28A1 | UBE2G2 | REG1B | COL6A3 | TNFRSF1A | | | 0.7816797 |

TABLE 9f

| X1 | X2 | X3 | X4 | X5 | X6 | X7 | AUC |
|---|---|---|---|---|---|---|---|
| COL28A1 | UBE2G2 | REG1B | COL6A3 | HAVCR1 | FSTL3 | | 0.80963073 |
| COL28A1 | UBE2G2 | REG1B | COL6A3 | HAVCR1 | RGMB | | 0.80991852 |
| COL28A1 | UBE2G2 | REG1B | COL6A3 | HAVCR1 | REG1A | | 0.79725153 |
| COL28A1 | UBE2G2 | REG1B | COL6A3 | HAVCR1 | CST3 | | 0.80121623 |
| COL28A1 | UBE2G2 | REG1B | COL6A3 | HAVCR1 | TNFRSF1A | | 0.81164528 |
| COL28A1 | UBE2G2 | REG1B | HAVCR1 | TNFRSF1A | FSTL3 | | 0.81652554 |
| COL28A1 | UBE2G2 | REG1B | HAVCR1 | TNFRSF1A | RGMB | | 0.8182148 |
| COL28A1 | UBE2G2 | REG1B | HAVCR1 | TNFRSF1A | REG1A | | 0.81293342 |
| COL28A1 | UBE2G2 | REG1B | HAVCR1 | TNFRSF1A | CST3 | | 0.81364272 |
| COL28A1 | UBE2G2 | REG1B | HAVCR1 | TNFRSF1A | TNFRSF1A | | 0.81340058 |
| COL28A1 | UBE2G2 | REG1B | TNFRSF1A | FSTL3 | RGMB | | 0.79228648 |
| COL28A1 | UBE2G2 | REG1B | TNFRSF1A | FSTL3 | REG1A | | 0.7887017 |
| COL28A1 | UBE2G2 | REG1B | TNFRSF1A | FSTL3 | CST3 | | 0.78898542 |
| COL28A1 | UBE2G2 | REG1B | TNFRSF1A | FSTL3 | TNFRSF1A | | 0.78897808 |
| COL28A1 | UBE2G2 | REG1B | FSTL3 | REG1A | RGMB | | 0.77836967 |
| COL28A1 | UBE2G2 | REG1B | FSTL3 | REG1A | CST3 | | 0.77037178 |
| COL28A1 | UBE2G2 | REG1B | FSTL3 | REG1A | TNFRSF1A | | 0.7887017 |

### Example 2. Datasets for Test Development and Validation

2.1 Development and validation cohort(s). CRIC is a multi-site observational study initiated to explore the relationship between chronic renal insufficiency and cardiovascular disease and has since expanded to measure many outcomes that are thought to be associated with renal insufficiency such as cognitive decline and frailty. CRIC enrolled patients ages 21 to 74 years of age, half of whom have diabetes mellitus. Participants had annual in-person follow-up visits (where urine and plasma were collected and stored) and telephone interviews every 6 months, where study outcomes and general health status were ascertained. Study recruitment began in 2003 and recruitment lasted for about 2.5 years at 13 clinical sites in the United States; investigators continue to monitor this cohort. The SomaLogic CRIC dataset includes clinical data and second annual visit samples (collected July 2003 through December 2009) for 3413 participants with kidney disease who were not yet experiencing end stage renal disease by the second annual visit.
2.2 Dataset Stratification. For this test, the cohort was split independently into training (70%), verification (15%), and validation (15%) sets, allowing identification of a robust model while mitigating overfitting issues. The validation data set was not used in the POC or refinement stages.

### 2.2.1 Model Development Data

TABLE 10
Demographic data for training dataset.

| Covariate | Measure | Total | |
|---|---|---|---|
| Sample size | n | 2315 | |
| Age | Mean (SD) | 58.8 | (10.7) |
| | Median | 60 | |
| | Range | 22.0-76.0 | |
| Gender‡ | Male | 1270 | (56%) |
| | Female | 999 | (44%) |
| Ethnicity | Caucasian | 1005 | (43%) |
| | Black | 933 | (40%) |
| | Other | 377 | (17%) |
| Proteinuria (g/24 hrs) | Mean (SD) | 1.0 | (2.1) |
| | Median | 0.2 | |
| | Range | 0.0-29.5 | |
| Diabetes | Yes | 1158 | (50%) |
| | No | 1157 | (50%) |
| eGFR at Baseline | Mean (SD) | 42.6 | (16.7) |
| (mL/min/1.73 m\hat{ }2)* | Median | 41.2 | |
| | Range | 7.8-117.0 | |
| | >=60 | 372 | (16%) |
| | <60 | 1898 | (84%) |
| PCRI at 4 years | Yes | 821 | (34%) |
| | No | 1494 | (66%) |
| eGFR at 4 years (IE6) | Mean (SD) | 45.5 | (19.8) |
| (mL/min/1.73 m\hat{ }2)* | Median | 43.4 | |
| | Range | 5.9-118.5 | |
| eGFR at 6 years (IE8) | Mean (SD) | 46.0 | (19.8) |
| (mL/min/1.73 m\hat{ }2) | Median | 44.7 | |
| | Range | 4.7-149.7 | |
| CKD Stage at Baseline† | Stage I | 10 | (0.4%) |
| | Stage II | 371 | (16%) |
| | Stage IIIa | 561 | (24%) |
| | Stage IIIb | 766 | (33%) |
| | Stage IV | 501 | (22%) |
| | Stage V | 63 | (3%) |

‡n missing = 46 (2%)
*n missing = 45 (2%)
†n missing = 43 (2%)

TABLE 11
Demographic data for verification dataset.

| Covariate | Measure | Total | |
|---|---|---|---|
| Sample size | n | 496 | |
| Age | Mean (SD) | 59.8 | (10.8) |
| | Median | 62 | |
| | Range | 22.0-76.0 | |
| Gender | Male | 266 | (54%) |
| | Female | 230 | (46%) |
| Ethnicity | Caucasian | 201 | (41%) |
| | Black | 207 | (42%) |
| | Other | 88 | (17%) |
| Proteinuria (g/24 hrs) | Mean (SD) | 0.85 | (1.65) |
| | Median | 0.1 | |
| | Range | 0.0-13.8 | |
| Diabetes‡ | Yes | 241 | (50%) |
| | No | 245 | (50%) |
| eGFR at Baseline* | Mean (SD) | 42.5 | (16.3) |
| (mL/min/1.73 m\hat{ }2) | Median | 41.3 | |
| | Range | 9.2-90.4 | |
| | >=60 | 78 | (16%) |
| | <60 | 409 | (84%) |
| PCRI at 4 years | Yes | 176 | (35%) |
| | No | 320 | (65%) |
| eGFR at 4 years (IE6) | Mean (SD) | 45.3 | (19.8) |
| (mL/min/1.73 m\hat{ }2) | Median | 42.4 | |
| | Range | 8.3-106.0 | |
| eGFR at 6 years (IE8) | Mean (SD) | 46.2 | (18.9) |
| (mL/min/1.73 m\hat{ }2) | Median | 46.1 | |
| | Range | 7.4-114.8 | |
| CKD Stage at Baseline† | Stage I | 2 | (0.5%) |
| | Stage II | 77 | (16%) |
| | Stage IIIa | 123 | (25%) |
| | Stage IIIb | 154 | (31%) |
| | Stage IV | 119 | (24%) |
| | Stage V | 12 | (2.5%) |

‡n missing = 10 (2%)
*n missing = 9 (2%)
†tn missing = 9 (2%)

### 2.2.2 Model Validation Data

TABLE 12
Demographic data for validation dataset

| Covariate | Measure | Total | |
|---|---|---|---|
| Sample size | n | 494 | |
| Age | Mean (SD) | 58.86 | (10.4) |
| | Median | 60 | |
| | Range | 23-75 | |
| Gender | Male | 278 | (56%) |
| | Female | 216 | (44%) |
| Ethnicity | Caucasian | 223 | (45%) |
| | Black | 200 | (40%) |
| | Other | 71 | (15%) |
| Proteinuria (g/24 hrs) | Mean (SD) | 0.97 | (2.13) |
| | Median | 0.2 | |
| | Range | 0.0-18.4 | |
| Diabetes | Yes | 244 | (49%) |
| | No | 250 | (51%) |
| eGFR at Baseline* | Mean (SD) | 42.3 | (15.8) |
| (mL/min/1.73 m\hat{ }2) | Median | 41.6 | |
| | Range | 10.6-86.4 | |
| | >=60 | 69 | (14%) |
| | <60 | 411 | (86%) |
| PCRI at 4 years | Yes | 175 | (35%) |
| | No | 319 | (65%) |
| eGFR at 4 years (IE6) | Mean (SD) | 45.2 | (20.8) |
| (mL/min/1.73 m\hat{ }2) | Median | 41 | |
| | Range | 9.5-108.3 | |
| eGFR at 6 years (IE8) | Mean (SD) | 42.4 | (20.6) |
| (mL/min/1.73 m\hat{ }2) | Median | 39.4 | |
| | Range | 8.3-99.5 | |
| CKD Stage at Baseline† | Stage I | 0 | (0%) |
| | Stage II | 69 | (14%) |
| | Stage IIIa | 131 | (27%) |
| | Stage IIIb | 162 | (33%) |
| | Stage IV | 109 | (22%) |
| | Stage V | 8 | (1.8%) |

*n missing = 20 (4%)
†n missing = 21 (4%)

### Example 3. Results from Development

3.1 Data QC and Pre-Analytics Results. Previous data QC and a feasibility POC has been conducted on the CRIC cohort for this endpoint. The samples for this test were run on assay version 4.0 from Jan. 21, 2019 until Sep. 30, 2019.
3.2 POC Approach and Results. The model that performed the best was a logistic regression model, which exceeded the passing criterion of an AUC≥0.65, 0.7, 0.75, or 0.77.
3.3 Refinement Approach and Results. The initial features used were the top 200 aptamers from univariate results, sorted by rank. Only features with greater than 0.75 correlation between assay versions 4.0 and 4.1 were used in model development in an effort to increase model flexibility across assay versions. (Actual correlation values for the final 10 features in the model are in Table 13). The feature list was further refined through repeated use of elastic net logistic regression—after each round of elastic net regression, features with absolute value of their coefficients below a threshold value were dropped, and the model was re-fit. This threshold was initially set at 0.01 and increased over iterations to 0.05. When the final feature set was selected, an un-penalized logistic regression model was fit with the remaining 10 features.

TABLE 13
CCC between assay version 4.0 and 4.1
for the 10 features in the final model.

| Feature Name | CCC |
|---|---|
| COL28A1 | 0.924 |
| COL6A3 | 0.868 |
| REG1A | 0.906 |
| REG1B | 0.951 |
| CST3 | 0.856 |
| TNFRSF1A | 0.832 |
| RGMB | 0.837 |
| FSTL3 | 0.871 |
| HAVCR1 | 0.954 |
| UBE2G2 | 0.805 |

The final model chosen is a logistic regression model with 10 features, which achieved an AUC of 0.82 on both the training and verification data. The equation for the logistic regression model is:

probability of CKD=inv.logit(intercept+sum(coefficient*feature level))

For this model, the intercept=−0.803923, and the feature names and coefficients are shown in Table 8. The feature level is the RFU (relative fluorescence units) as measured in a sample by a proteomic assay, for example, an aptamer-based assay. The KFRE was used to evaluate comparative performance; it achieved an AUC of 0.77 (95% CI: 0.75, 0.78) on the entire CRIC cohort. Table 14 shows the AUC and 95% CI for training and verification data from the final model.

TABLE 14
AUC and 95% CI for training and verification data from the final model

| | | | Model | AUC | | | Balanced |
|---|---|---|---|---|---|---|---|
| Dataset | n | KFRE AUC | AUC | (95% CI) | Sensitivity | Specificity | Accuracy |
| Training | 2315 | 0.77 | 0.82 | (0.80, 0.84) | 0.747 | 0.759 | 0.753 |
| Verification | 496 | (95% CI | 0.82 | (0.78, 0.86) | 0.744 | 0.756 | 0.75 |
| | | 0.75, 0.78) | | | | | |

The optimal decision threshold was determined by maximizing the F1 score, which is the harmonic mean of sensitivity and specificity, and was found at p=0.3533. This probability was used as the threshold to decide between a “yes” or “no” status for developing PCRI within the next four years. Predicted probabilities <0.3533 are labeled as “no” and predicted probabilities >0.3533 are labelled as “yes.” Also included were tests for model robustness in the refinement process. In some embodiments, p=0.3, 0.31, 0.32, 0.33, 0.34, 0.35 or 0.3533.

TABLE 15
Probability and relative risk ranges used to determine quintiles

| Quintile | probability range | relative risk range |
|---|---|---|
| 1 | ≤0.1864 | [0.0, 0.36] |
| 2 | (0.1865, 0.3361] | (0.36, 0.7] |
| 3 | (0.3361, 0.5219] | (0.7, 1.21] |
| 4 | (0.5219, 0.7365] | (1.21, 1.95] |
| 5 | >0.7365* | (1.95, 3.24] |

*0.9999 is the highest possible probability score

### Example 4. Model Validation Plan and Results

4.1 Clinical Validation Plan. The model was validated by calculating the AUC for the PCRI yes/no at four years endpoint on the 15% hold out validation dataset. Acceptance criteria was that the AUC was greater than or equal to 0.65, 0.7, 0.75, or 0.77

Clinical Results on Validation Data. The required passing criterion was that the AUC of our model in the full validation cohort have at least the same AUC as the KFRE in the full CRIC cohort (AUC>0.77), Validation results are in Table 19. See FIG. 3.

TABLE 19
AUC and 95% CI on training, verification and validation cohorts.

| | | | Model | AUC | | | |
|---|---|---|---|---|---|---|---|
| Dataset | n | KFRE AUC | AUC | (95% CI) | Sensitivity | Specificity | Accuracy |
| Training | 2315 | 0.77 | 0.82 | (0.80, 0.84) | 0.747 | 0.759 | 0.754 |
| Verification | 496 | (95% CI | 0.82 | (0.78, 0.86) | 0.744 | 0.756 | 0.752 |
| Validation | 494 | 0.75, 0.78) | 0.79 | (0.74, 0.82) | 0.743 | 0.718 | 0.727 |

### REFERENCES

All references listed below, or anywhere else throughout this description, are hereby incorporated by reference herein in their entireties:
1. Hannan M, Ansari S, Meza N, et al. Risk Factors for CKD Progression: Overview of Findings from the CRIC Study. Clin J Am Soc Nephrol 2020; 11:07830520.
2. Feldman H I, Appel L J, Chertow G M, et al. The Chronic Renal Insufficiency Cohort (CRIC) Study: Design and Methods. J Am Soc Nephrol 2003; 14:S148-153.
3. National Institutes of Health NIoDaDaKD. 2019 USRDS Annual Data Report: Epidemiology of Kidney Disease in the United States. 2019:64.
4. Levey A S, Coresh J. Chronic kidney disease. Lancet 2012; 379:165-180. doi: 110.1016/S0140-6736(1011)60178-60175. Epub 6211 August 60115.
5. Chapter 4: Other complications of CKD: CVD, medication dosage, patient safety, infections, hospitalizations, and caveats for investigating complications of CKD. 2011) 2013; 3:91-111. doi: 110.1038/kisup.2012.1067.
6. Chapter 3: Management of progression and complications of CKD. 2011) 2013; 3:73-90. doi: 10.1038/kisup.2012.1066.
7. Chapter 2: Definition, identification, and prediction of CKD progression. 2011) 2013; 3:63-72. doi: 10.1038/kisup.2012.1065.
8. Tangri N, Stevens L A, Griffith J, et al. A predictive model for progression of chronic kidney disease to kidney failure. JAMA 2011; 305:1553-1559. doi: 1510.1001/jama.2011.1451. Epub 211 April 1511.
9. Centers for Disease Control and Prevention. Chronic Kidney Disease Surveillance System-United States. http://www.cdc.gov/ckd, 2021.
10. Major R W, Shepherd D, Medcalf J F, et al. The Kidney Failure Risk Equation for prediction of end stage renal disease in U K primary care: An external validation and clinical impact projection cohort study. PLoS Med 2019; 16:e1002955. doi: 1002910.1001371/journal.pmed.1002955. eCollection 1002019 November
11. Lash J P, Go A S, Appel L J, et al. Chronic Renal Insufficiency Cohort (CRIC) Study: baseline characteristics and associations with kidney function. Clin J Am Soc Nephrol 2009; 4:1302-1311.
12. Fuentes A V, Pineda M D, Venkata K C N. Comprehension of Top 200 Prescribed Drugs in the U S as a Resource for Pharmacy Teaching, Training and Practice. Pharmacy (Basel) 2018; 6:43. doi: 10.3390/pharmacy6020043.
13. Song, et al. (2019) “Understanding kidney injury molecule 1: a novel immune factor in kidney pathophysiology” Am J Transl Res 11(3): 1219-1229
14. Kralisch, et al. (2017) “FSTL3 is increased in renal dysfunction” Nephrol Dial Transplant 32: 1637-1644
15. Liu, et al. (2018) “RGMb protects against acute kidney injury by inhibiting tubular cell necroptosis via an MLKL-dependent mechanism” Proc Natl Acad Sci USA 115(7): E1475-E1484
16. Li, et al. (2017) “Elevated serum level of pancreatic stone protein/regenerating protein (PSP/reg) is observed in diabetic kidney disease” Oncotarget 8(24): 38145-38151
17. Yamada, et al. (2018) “Identification of 13 novel susceptibility loci for early-onset myocardial infarction, hypertension, or chronic kidney disease” Int J Mol Med 42(5): 2415-2436
18. Peralta, et al. (2011) “Cystatin C identifies chronic kidney disease patients at higher risk for complications” J Am Soc Nephrol 22(1): 147-155
19. Nadkarni, et al. (2018) “Plasma biomarkers are associated with renal outcomes in individuals with APOL1 risk variants” Kidney Int. 93(6): 1409-1416
20. Garrod, et al. (1990) “Early expression of desmosomal components during kidney tubule morphogenesis in human and murine embryos” 108(2): 313-321
21. Mohammed, et al. (2019) “Cardiac Biomarkers and Cardiovascular Outcome in Children with Chronic Kidney Disease” Iran J Kidney Dis. 13(2): 120-128
22. Schneider, et al. (1994) “Human cyclophilin C: primary structure, tissue distribution, and determination of binding specificity for cyclosporins” Biochemistry 33(27): 8218-8224
23. Friedman, et al. (1994) “An analysis of the expression of cyclophilin C reveals tissue restriction and an intriguing pattern in the mouse kidney” Am J Pathol. 144(6): 1247-56
24. Jantti, et al. (Aug. 28, 2020) “Trophic activities of endoplasmic reticulum proteins CDNF and MANF” Cell and Tissue Research 382: 83-100
25. Zhang, et al. (2019) “Follicle-stimulating hormone promotes renal tubulointerstitial fibrosis in aging women via the AKT/GSK-30/0-catenin pathway” Aging Cell 18(5): E12997
26. Tian, et al. (1992) “The variations of PRL, L H, FSH and testosterone in renal disease” Hua Xi Yi Ke Da Xue Xue Bao 23(1): 94-97
27. Koyun, et al. (2009) “Evaluation of reproductive functions in male adolescents following renal transplantation” Pediatr Transplant 13(6): 697-700
28. Colak, et al. (2014) “The relation between serum testosterone levels and cardiovascular risk factors in patients with kidney transplantation and chronic kidney disease” Saudi J Kidney Dis Transpl. 25(5): 951-959

What is claimed is:

1. A method comprising:
a. measuring the level of COL28A1 protein, and the level of at least one, two, three, four, five, six, seven, eight, or nine proteins selected from the group consisting of HAVCR1, FSTL3, RGMB, UBE2G2, REG1A, REG1B, COL6A3, CST3, and TNFRSF1A in a sample from a human subject; and
b. identifying the human subject as being at relative risk for developing progressive chronic renal insufficiency within a 4 year period based on the level of COL28A1 and the level of the at least one, two, three, four, five, six, seven, eight, or nine proteins.

2. A method comprising:
a. measuring the level of UBE2G2 protein, and the level of at least one, two, three, four, five, six, seven, eight, or nine proteins selected from the group consisting of HAVCR1, FSTL3, RGMB, COL28A1, REG1A, REG1B, COL6A3, CST3, and TNFRSF1A in a sample from a human subject; and
b. identifying the human subject as being at relative risk for developing progressive chronic renal insufficiency within a 4 year period based on the level of UBE2G2 and the level of the at least one, two, three, four, five, six, seven, eight, or nine proteins.

3. A method comprising:
a. measuring the level of REG1B protein, and the level of at least one, two, three, four, five, six, seven, eight, or nine proteins selected from the group consisting of HAVCR1, FSTL3, RGMB, COL28A1, UBE2G2, REG1A, COL6A3, CST3, and TNFRSF1A in a sample from a human subject; and
b. identifying the human subject as being at relative risk for developing progressive chronic renal insufficiency within a 4 year period based on the level of REG1B and the level of the at least one, two, three, four, five, six, seven, eight, or nine proteins.

4. The method of claim 1, wherein the method comprises measuring COL28A1 and HAVCR1, COL28A1 and FSTL3, COL28A1 and RGMB, COL28A1 and UBE2G2, COL28A1 and REG1A, COL28A1 and REG1B, COL28A1 and COL6A3, COL28A1 and CST3, or COL28A1 and TNFRSF1A.

5. The method of claim 1, wherein the method comprises measuring COL28A1, HAVCR1, and FSTL3; COL28A1, HAVCR1, and RGMB; COL28A1, HAVCR1, and UBE2G2; COL28A1, HAVCR1, and REG1A; COL28A1, HAVCR1, and REG1B; COL28A1, HAVCR1, and COL6A3; COL28A1, HAVCR1, and CST3; COL28A1, HAVCR1, and TNFRSF1A; COL28A1, FSTL3, and RGMB; COL28A1, FSTL3, and UBE2G2; COL28A1, FSTL3, and REG1A; COL28A1, FSTL3, and REG1B; COL28A1, FSTL3, and COL6A3; COL28A1, FSTL3, and CST3; COL28A1, FSTL3, and TNFRSF1A; COL28A1, RGMB, and UBE2G2; COL28A1, RGMB, and REG1A; COL28A1, RGMB, and REG1B; COL28A1, RGMB, and COL6A3; COL28A1, RGMB, and CST3; COL28A1, RGMB, and TNFRSF1A; COL28A1, UBE2G2, and REG1A; COL28A1, UBE2G2, and REG1B; COL28A1, UBE2G2, and COL6A3; COL28A1, UBE2G2, and CST3; COL28A1, UBE2G2, and TNFRSF1A; COL28A1, REG1A, and REG1B; COL28A1, REG1A, and COL6A3; COL28A1, REG1A, and CST3; COL28A1, REG1A, and TNFRSF1A; COL28A1, REG1B, and COL6A3; COL28A1, REG1B, and CST3; COL28A1, REG1B, and TNFRSF1A; COL28A1, COL6A3, and CST3; COL28A1, COL6A3, and TNFRSF1A; or COL28A1, CST3, and TNFRSF1A.

6. The method of claim 2, wherein the method comprises measuring UBE2G2 and HAVCR1, UBE2G2 and FSTL3, UBE2G2 and RGMB, UBE2G2 and COL28A1, UBE2G2 and REG1A, UBE2G2 and REG1B, UBE2G2 and COL6A3, UBE2G2 and CST3, or UBE2G2 and TNFRSF1A.

7. The method of claim 2, wherein the method comprises measuring UBE2G2, HAVCR1, and FSTL3; UBE2G2, HAVCR1, and RGMB; UBE2G2, HAVCR1, and COL28A1; UBE2G2, HAVCR1, and REG1A; UBE2G2, HAVCR1, and REG1B; UBE2G2, HAVCR1, and COL6A3; UBE2G2, HAVCR1, and CST3; UBE2G2, HAVCR1, and TNFRSF1A; UBE2G2, FSTL3, and RGMB; UBE2G2, FSTL3, and COL28A1; UBE2G2, FSTL3, and REG1A; UBE2G2, FSTL3, and REG1B; UBE2G2, FSTL3, and COL6A3; UBE2G2, FSTL3, and CST3; UBE2G2, FSTL3, and TNFRSF1A; UBE2G2, RGMB, and COL28A1; UBE2G2, RGMB, and REG1A; UBE2G2, RGMB, and REG1B; UBE2G2, RGMB, and COL6A3; UBE2G2, RGMB, and CST3; UBE2G2, RGMB, and TNFRSF1A; UBE2G2, COL28A1, and REG1A; UBE2G2, COL28A1, and REG1B; UBE2G2, COL28A1, and COL6A3; UBE2G2, COL28A1, and CST3; UBE2G2, COL28A1, and TNFRSF1A; UBE2G2, REG1A, and REG1B; UBE2G2, REG1A, and COL6A3; UBE2G2, REG1A, and CST3; UBE2G2, REG1A, and TNFRSF1A; UBE2G2, REG1B, and COL6A3; UBE2G2, REG1B, and CST3; UBE2G2, REG1B, and TNFRSF1A; UBE2G2, COL6A3, and CST3; UBE2G2, COL6A3, and TNFRSF1A; or UBE2G2, CST3, and TNFRSF1A.

8. The method of claim 3, wherein the method comprises measuring REG1B and HAVCR1, REG1B and FSTL3, REG1B and RGMB, REG1B and COL28A1, REG1B and UBE2G2, REG1B and REG1A, REG1B and COL6A3, REG1B and CST3, or REG1B and TNFRSF1A.

9. The method of claim 3, wherein the method comprises measuring REG1B, HAVCR1, and FSTL3; REG1B, HAVCR1, and RGMB; REG1B, HAVCR1, and COL28A1; REG1B, HAVCR1, and UBE2G2; REG1B, HAVCR1, and REG1A; REG1B, HAVCR1, and COL6A3; REG1B, HAVCR1, and CST3; REG1B, HAVCR1, and TNFRSF1A; REG1B, FSTL3, and RGMB; REG1B, FSTL3, and COL28A1; REG1B, FSTL3, and UBE2G2; REG1B, FSTL3, and REG1A; REG1B, FSTL3, and COL6A3; REG1B, FSTL3, and CST3; REG1B, FSTL3, and TNFRSF1A; REG1B, RGMB, and COL28A1; REG1B, RGMB, and UBE2G2; REG1B, RGMB, and REG1A; REG1B, RGMB, and COL6A3; REG1B, RGMB, and CST3; REG1B, RGMB, and TNFRSF1A; REG1B, COL28A1, and UBE2G2; REG1B, COL28A1, and REG1A; REG1B, COL28A1, and COL6A3; REG1B, COL28A1, and CST3; REG1B, COL28A1, and TNFRSF1A; REG1B, UBE2G2, and REG1A; REG1B, UBE2G2, and COL6A3; REG1B, UBE2G2, and CST3; REG1B, UBE2G2, and TNFRSF1A; REG1B, REG1A, and COL6A3; REG1B, REG1A, and CST3; REG1B, REG1A, and TNFRSF1A; REG1B, COL6A3, and CST3; REG1B, COL6A3, and TNFRSF1A; or REG1B, CST3, and TNFRSF1A.

10. The method of claim 1, wherein the method comprises measuring COL28A1 and UBE2G2, and at least one of the following proteins selected from HAVCR1, FSTL3, RGMB, REG1A, REG1B, COL6A3, CST3, and TNFRSF1A.

11. The method of claim 1, wherein the method comprises measuring COL28A1 and REG1B, and at least one of the following proteins selected from HAVCR1, FSTL3, RGMB, UBE2G2, REG1A, COL6A3, CST3, and TNFRSF1A.

12. The method of claim 2, wherein the method comprises measuring UBE2G2 and REG1B, and at least one of the following proteins selected from HAVCR1, FSTL3, RGMB, COL28A1, REG1A, COL6A3, CST3, and TNFRSF1A.

13. The method of any one of claims 1 to 12, wherein progressive chronic renal insufficiency within a 4 year period indicates the development of one or more of a 50% decline in estimated glomerular filtration rate (eGFR), a diagnosis that kidney dialysis is needed, development of eGFR<15 ml/min/1.73 m², development of end stage renal disease (ESRD) or a diagnosis that a kidney transplantation is needed.

14. The method of any one of claims 1 to 13, wherein the measuring is performed using mass spectrometry, an aptamer based assay and/or an antibody based assay.

15. The method of any one of claims 1 to 14, wherein the sample is selected from blood, plasma, serum or urine.

16. A method comprising:
a. contacting a sample from a human subject with a set of capture reagents, wherein each capture reagent has affinity for a different protein of the set of proteins comprising COL28A1 protein, and at least one, two, three, four, five, six, seven, eight, or nine proteins selected from the group consisting of HAVCR1, FSTL3, RGMB, UBE2G2, REG1A, REG1B, COL6A3, CST3, and TNFRSF1A; and
b. measuring the level of each protein of the set of proteins with the set of capture reagents.

17. A method comprising:
a. contacting a sample from a human subject with a set of capture reagents, wherein each capture reagent has affinity for a different protein of the set of proteins comprising UBE2G2 protein, and at least one, two, three, four, five, six, seven, eight, or nine proteins selected from the group consisting of HAVCR1, FSTL3, RGMB, COL28A1, REG1A, REG1B, COL6A3, CST3, and TNFRSF1A; and
b. measuring the level of each protein of the set of proteins with the set of capture reagents.

18. A method comprising:
a. contacting a sample from a human subject with a set of capture reagents, wherein each capture reagent has affinity for a different protein of the set of proteins comprising REG1B protein, and at least one, two, three, four, five, six, seven, eight, or nine proteins selected from the group consisting of HAVCR1, FSTL3, RGMB, COL28A1, UBE2G2, REG1A, COL6A3, CST3, and TNFRSF1A; and
b. measuring the level of each protein of the set of proteins with the set of capture reagents.

19. The method of claim 16, wherein the method comprises measuring COL28A1 and HAVCR1, COL28A1 and FSTL3, COL28A1 and RGMB, COL28A1 and UBE2G2, COL28A1 and REG1A, COL28A1 and REG1B, COL28A1 and COL6A3, COL28A1 and CST3, or COL28A1 and TNFRSF1A.

20. The method of claim 16, wherein the method comprises measuring COL28A1, HAVCR1, and FSTL3; COL28A1, HAVCR1, and RGMB; COL28A1, HAVCR1, and UBE2G2; COL28A1, HAVCR1, and REG1A; COL28A1, HAVCR1, and REG1B; COL28A1, HAVCR1, and COL6A3; COL28A1, HAVCR1, and CST3; COL28A1, HAVCR1, and TNFRSF1A; COL28A1, FSTL3, and RGMB; COL28A1, FSTL3, and UBE2G2; COL28A1, FSTL3, and REG1A; COL28A1, FSTL3, and REG1B; COL28A1, FSTL3, and COL6A3; COL28A1, FSTL3, and CST3; COL28A1, FSTL3, and TNFRSF1A; COL28A1, RGMB, and UBE2G2; COL28A1, RGMB, and REG1A; COL28A1, RGMB, and REG1B; COL28A1, RGMB, and COL6A3; COL28A1, RGMB, and CST3; COL28A1, RGMB, and TNFRSF1A; COL28A1, UBE2G2, and REG1A; COL28A1, UBE2G2, and REG1B; COL28A1, UBE2G2, and COL6A3; COL28A1, UBE2G2, and CST3; COL28A1, UBE2G2, and TNFRSF1A; COL28A1, REG1A, and REG1B; COL28A1, REG1A, and COL6A3; COL28A1, REG1A, and CST3; COL28A1, REG1A, and TNFRSF1A; COL28A1, REG1B, and COL6A3; COL28A1, REG1B, and CST3; COL28A1, REG1B, and TNFRSF1A; COL28A1, COL6A3, and CST3; COL28A1, COL6A3, and TNFRSF1A; or COL28A1, CST3, and TNFRSF1A.

21. The method of claim 17, wherein the method comprises measuring UBE2G2 and HAVCR1, UBE2G2 and FSTL3, UBE2G2 and RGMB, UBE2G2 and COL28A1, UBE2G2 and REG1A, UBE2G2 and REG1B, UBE2G2 and COL6A3, UBE2G2 and CST3, or UBE2G2 and TNFRSF1A.

22. The method of claim 17, wherein the method comprises measuring UBE2G2, HAVCR1, and FSTL3; UBE2G2, HAVCR1, and RGMB; UBE2G2, HAVCR1, and COL28A1; UBE2G2, HAVCR1, and REG1A; UBE2G2, HAVCR1, and REG1B; UBE2G2, HAVCR1, and COL6A3; UBE2G2, HAVCR1, and CST3; UBE2G2, HAVCR1, and TNFRSF1A; UBE2G2, FSTL3, and RGMB; UBE2G2, FSTL3, and COL28A1; UBE2G2, FSTL3, and REG1A; UBE2G2, FSTL3, and REG1B; UBE2G2, FSTL3, and COL6A3; UBE2G2, FSTL3, and CST3; UBE2G2, FSTL3, and TNFRSF1A; UBE2G2, RGMB, and COL28A1; UBE2G2, RGMB, and REG1A; UBE2G2, RGMB, and REG1B; UBE2G2, RGMB, and COL6A3; UBE2G2, RGMB, and CST3; UBE2G2, RGMB, and TNFRSF1A; UBE2G2, COL28A1, and REG1A; UBE2G2, COL28A1, and REG1B; UBE2G2, COL28A1, and COL6A3; UBE2G2, COL28A1, and CST3; UBE2G2, COL28A1, and TNFRSF1A; UBE2G2, REG1A, and REG1B; UBE2G2, REG1A, and COL6A3; UBE2G2, REG1A, and CST3; UBE2G2, REG1A, and TNFRSF1A; UBE2G2, REG1B, and COL6A3; UBE2G2, REG1B, and CST3; UBE2G2, REG1B, and TNFRSF1A; UBE2G2, COL6A3, and CST3; UBE2G2, COL6A3, and TNFRSF1A; or UBE2G2, CST3, and TNFRSF1A.

23. The method of claim 18, wherein the method comprises measuring REG1B and HAVCR1, REG1B and FSTL3, REG1B and RGMB, REG1B and COL28A1, REG1B and UBE2G2, REG1B and REG1A, REG1B and COL6A3, REG1B and CST3, or REG1B and TNFRSF1A.

24. The method of claim 18, wherein the method comprises measuring REG1B, HAVCR1, and FSTL3; REG1B, HAVCR1, and RGMB; REG1B, HAVCR1, and COL28A1; REG1B, HAVCR1, and UBE2G2; REG1B, HAVCR1, and REG1A; REG1B, HAVCR1, and COL6A3; REG1B, HAVCR1, and CST3; REG1B, HAVCR1, and TNFRSF1A; REG1B, FSTL3, and RGMB; REG1B, FSTL3, and COL28A1; REG1B, FSTL3, and UBE2G2; REG1B, FSTL3, and REG1A; REG1B, FSTL3, and COL6A3; REG1B, FSTL3, and CST3; REG1B, FSTL3, and TNFRSF1A; REG1B, RGMB, and COL28A1; REG1B, RGMB, and UBE2G2; REG1B, RGMB, and REG1A; REG1B, RGMB, and COL6A3; REG1B, RGMB, and CST3; REG1B, RGMB, and TNFRSF1A; REG1B, COL28A1, and UBE2G2; REG1B, COL28A1, and REG1A; REG1B, COL28A1, and COL6A3; REG1B, COL28A1, and CST3; REG1B, COL28A1, and TNFRSF1A; REG1B, UBE2G2, and REG1A; REG1B, UBE2G2, and COL6A3; REG1B, UBE2G2, and CST3; REG1B, UBE2G2, and TNFRSF1A; REG1B, REG1A, and COL6A3; REG1B, REG1A, and CST3; REG1B, REG1A, and TNFRSF1A; REG1B, COL6A3, and CST3; REG1B, COL6A3, and TNFRSF1A; or REG1B, CST3, and TNFRSF1A.

25. The method of claim 16, wherein the method comprises measuring COL28A1 and UBE2G2, and at least one of the following proteins selected from HAVCR1, FSTL3, RGMB, REG1A, REG1B, COL6A3, CST3, and TNFRSF1A.

26. The method of claim 16, wherein the method comprises measuring COL28A1 and REG1B, and at least one of the following proteins selected from HAVCR1, FSTL3, RGMB, UBE2G2, REG1A, COL6A3, CST3, and TNFRSF1A.

27. The method of claim 17, wherein the method comprises measuring UBE2G2 and REG1B, and at least one of the following proteins selected from HAVCR1, FSTL3, RGMB, COL28A1, REG1A, COL6A3, CST3, and TNFRSF1A.

28. The method of any one of claims 16 to 27, wherein the protein levels are used to identify a human subject as being at relative risk for developing progressive chronic renal insufficiency within a 4 year period.

29. The method of claim 28, wherein progressive chronic renal insufficiency within a 4 year period indicates the development of one or more of a 50% decline in estimated glomerular filtration rate (eGFR), a diagnosis that kidney dialysis is needed, development of eGFR<15 ml/min/1.73 m², development of end stage renal disease (ESRD), or a diagnosis that a kidney transplantation is needed.

30. The method of any one of claims 16 to 29, wherein the set of capture reagents is selected from aptamers, antibodies and a combinations of aptamers and antibodies.

31. The method of any one of claims 16 to 30, wherein the sample is selected from blood, plasma, serum or urine.

32. A method comprising:
a) contacting a sample from a human subject with two capture reagents, wherein one capture reagent has affinity for a COL28A1 protein and the second capture reagent has affinity for a UBE2G2 protein; and
b) measuring the level of each protein with the two capture reagents.

33. A method comprising:
a) contacting a sample from a human subject with two capture reagents, wherein one capture reagent has affinity for a COL28A1 protein and the second capture reagent has affinity for a REG1B protein; and
b) measuring the level of each protein with the two capture reagents.

34. A method comprising:
a) contacting a sample from a human subject with two capture reagents, wherein one capture reagent has affinity for a UBE2G2 protein and the second capture reagent has affinity for a REG1B protein; and
b) measuring the level of each protein with the two capture reagents.

35. The method of any one of claims 32 to 34, further comprising measuring the level of a HAVCR1 protein with a capture reagent having affinity for the HAVCR1 protein.

36. The method of any one of claims 32 to 35, further comprising measuring the level of a FSTL3 protein with a capture reagent having affinity for the FSTL3 protein.

37. The method of any one of claims 32 to 36, further comprising measuring the level of a RGMB protein with a capture reagent having affinity for the RGMB protein.

38. The method of any one of claims 32 to 37, further comprising measuring the level of a REG1A protein with a capture reagent having affinity for the REG1A protein.

39. The method of any one of claims 32 to 38, further comprising measuring the level of a COL6A3 protein with a capture reagent having affinity for the COL6A3 protein.

40. The method of any one of claims 32 to 39, further comprising measuring the level of a CST3 protein with a capture reagent having affinity for the CST3 protein.

41. The method of any one of claims 32 to 40, further comprising measuring the level of a TNFRSF1A protein with a capture reagent having affinity for the TNFRSF1A protein.

42. A method comprising:
a) measuring the level of COL28A1 and UBE2G2 in a sample from a human subject; and
b) identifying the human subject as being at relative risk for developing progressive chronic renal insufficiency within a 4 year period based on the level of COL28A1 and UBE2G2.

43. A method comprising:
a) measuring the level of COL28A1 and REG1B in a sample from a human subject; and
b) identifying the human subject as being at relative risk for developing progressive chronic renal insufficiency within a 4 year period based on the level of COL28A1 and REG1B.

44. A method comprising:
a) measuring the level of UBE2G2 and REG1B in a sample from a human subject; and
b) identifying the human subject as being at relative risk for developing progressive chronic renal insufficiency within a 4 year period based on the level of UBE2G2 and REG1B.

45. The method of any one of claims 42 to 44, further comprising measuring the level of a HAVCR1 protein.

46. The method of any one of claims 42 to 45, further comprising measuring the level of a FSTL3 protein.

47. The method of any one of claims 42 to 46, further comprising measuring the level of a RGMB protein.

48. The method of any one of claims 42 to 47, further comprising measuring the level of a REG1A protein.

49. The method of any one of claims 42 to 48, further comprising measuring the level of a COL6A3 protein.

50. The method of any one of claims 42 to 49, further comprising measuring the level of a CST3 protein.

51. The method of any one of claims 42 to 50, further comprising measuring the level of a TNFRSF1A protein.

52. The method of any one of claims 42 to 51, wherein the measuring is performed using mass spectrometry, an aptamer based assay and/or an antibody based assay.

53. A method comprising:
a) contacting a sample from a human subject with three capture reagents, wherein each of the three capture reagents has affinity for a protein selected from COL28A1, UBE2G2 and REG1B; and
b) measuring the level of each protein with the three capture reagents.

54. The method of claim 53, further comprising measuring the level of a HAVCR1 protein with a capture reagent having affinity for the HAVCR1 protein.

55. The method of claim 53 or 54, further comprising measuring the level of a FSTL3 protein with a capture reagent having affinity for the FSTL3 protein.

56. The method of any one of claims 53 to 55, further comprising measuring the level of a RGMB protein with a capture reagent having affinity for the RGMB protein.

57. The method of any one of claims 53 to 56, further comprising measuring the level of a REG1A protein with a capture reagent having affinity for the REG1A protein.

58. The method of any one of claims 53 to 57, further comprising measuring the level of a COL6A3 protein with a capture reagent having affinity for the COL6A3 protein.

59. The method of any one of claims 53 to 58, further comprising measuring the level of a CST3 protein with a capture reagent having affinity for the CST3 protein.

60. The method of any one of claims 53 to 59, further comprising measuring the level of a TNFRSF1A protein with a capture reagent having affinity for the TNFRSF1A protein.

61. A method comprising:
a) measuring the level of COL28A1, UBE2G2 and REG1B in a sample from a human subject; and
b) identifying the human subject as being at relative risk for developing progressive chronic renal insufficiency within a 4 year period based on the level of COL28A1, UBE2G2 and REG1B.

62. The method of claim 61, further comprising measuring the level of a HAVCR1 protein.

63. The method of claim 61 or 62, further comprising measuring the level of a FSTL3 protein.

64. The method of any one of claims 61 to 63, further comprising measuring the level of a RGMB protein.

65. The method of any one of claims 61 to 64, further comprising measuring the level of a REG1A protein.

66. The method of any one of claims 61 to 65, further comprising measuring the level of a COL6A3 protein.

67. The method of any one of claims 61 to 66, further comprising measuring the level of a CST3 protein.

68. The method of any one of claims 61 to 67, further comprising measuring the level of a TNFRSF1A protein.

69. The method of any one of claims 61 to 68, wherein the measuring is performed using mass spectrometry, an aptamer based assay and/or an antibody based assay.

70. A method comprising:
a) measuring the level of at least three, four, five, six, seven, eight, nine or ten proteins selected from the group consisting of HAVCR1, FSTL3, RGMB, COL28A1, UBE2G2, REG1A, REG1B, COL6A3, CST3, and TNFRSF1A in a sample from a human subject; and
b) identifying the human subject as being at relative risk for developing progressive chronic renal insufficiency within a 4 year period based on the level of the at least three, four, five, six, seven, eight, nine or ten proteins.

71. The method of claim 70, wherein the measuring is performed using mass spectrometry, an aptamer based assay and/or an antibody based assay.

72. The method of claim 70 or 71, wherein the sample is selected from blood, plasma, serum or urine.

73. The method of any one of claims 70 to 72, wherein the method comprises measuring HAVCR1, FSTL3, and RGMB; HAVCR1, FSTL3, and COL28A1; HAVCR1, FSTL3, and UBE2G2; HAVCR1, FSTL3, and REG1A; HAVCR1, FSTL3, and REG1B; HAVCR1, FSTL3, and COL6A3; HAVCR1, FSTL3, and CST3; HAVCR1, FSTL3, and TNFRSF1; HAVCR1, RGMB, and COL28A1; HAVCR1, RGMB, and UBE2G2; HAVCR1, RGMB, and REG1A; HAVCR1, RGMB, and REG1B; HAVCR1, RGMB, and COL6A3; HAVCR1, RGMB, and CST3; HAVCR1, RGMB, and TNFRSF1A; HAVCR1, COL28A1, and UBE2G2; HAVCR1, COL28A1, and REG1A; HAVCR1, COL28A1, and REG1B; HAVCR1, COL28A1, and COL6A3; HAVCR1, COL28A1, and CST3; HAVCR1, COL28A1, and TNFRSF1A; HAVCR1, UBE2G2, and REG1A; HAVCR1, UBE2G2, and REG1B; HAVCR1, UBE2G2, and COL6A3; HAVCR1, UBE2G2, and CST3; HAVCR1, UBE2G2, and TNFRSF1A; HAVCR1, REG1A, and REG1B; HAVCR1, REG1A, and COL6A3; HAVCR1, REG1A, and CST3; HAVCR1, REG1A, and TNFRSF1A; HAVCR1, REG1B, and COL6A3; HAVCR1, REG1B, and CST3; HAVCR1, REG1B, and TNFRSF1A; HAVCR1, COL6A3, and CST3; HAVCR1, COL6A3, and TNFRSF1A; HAVCR1, CST3, and TNFRSF1A; FSTL3, RGMB, and COL28A1; FSTL3, RGMB, and UBE2G2; FSTL3, RGMB, and REG1A; FSTL3, RGMB, and REG1B; FSTL3, RGMB, and COL6A3; FSTL3, RGMB, and CST3; FSTL3, RGMB, and TNFRSF1A; FSTL3, COL28A1, and UBE2G2; FSTL3, COL28A1, and REG1A; FSTL3, COL28A1, and REG1B; FSTL3, COL28A1, and COL6A3; FSTL3, COL28A1, and CST3; FSTL3, COL28A1, and TNFRSF1A; FSTL3, UBE2G2, and REG1A; FSTL3, UBE2G2, and REG1B; FSTL3, UBE2G2, and COL6A3; FSTL3, UBE2G2, and CST3; FSTL3, UBE2G2, and TNFRSF1A; FSTL3, REG1A, and REG1B; FSTL3, REG1A, and COL6A3; FSTL3, REG1A, and CST3; FSTL3, REG1A, and TNFRSF1A; FSTL3, REG1B, and COL6A3; FSTL3, REG1B, and CST3; FSTL3, REG1B, and TNFRSF1A; FSTL3, COL6A3, and CST3; FSTL3, COL6A3, and TNFRSF1A; FSTL3, CST3, and TNFRSF1A; RGMB, COL28A1, and UBE2G2; RGMB, COL28A1, and REG1A; RGMB, COL28A1, and REG1B; RGMB, COL28A1, and COL6A3; RGMB, COL28A1, and CST3; RGMB, COL28A1, and TNFRSF1A; RGMB, UBE2G2, and REG1A; RGMB, UBE2G2, and REG1B; RGMB, UBE2G2, and COL6A3; RGMB, UBE2G2, and CST3; RGMB, UBE2G2, and TNFRSF1A; RGMB, REG1A, and REG1B; RGMB, REG1A, and COL6A3; RGMB, REG1A, and CST3; RGMB, REG1A, and TNFRSF1A; RGMB, REG1B, and COL6A3; RGMB, REG1B, and CST3; RGMB, REG1B, and TNFRSF1A; RGMB, COL6A3, and CST3; RGMB, COL6A3, and TNFRSF1A; RGMB, CST3, and TNFRSF1A; COL28A1, UBE2G2, and REG1A; COL28A1, UBE2G2, and REG1B; COL28A1, UBE2G2, and COL6A3; COL28A1, UBE2G2, and CST3; COL28A1, UBE2G2, and TNFRSF1A; COL28A1, REG1A, and REG1B; COL28A1, REG1A, and COL6A3; COL28A1, REG1A, and CST3; COL28A1, REG1A, and TNFRSF1A; COL28A1, REG1B, and COL6A3; COL28A1, REG1B, and CST3; COL28A1, REG1B, and TNFRSF1A; COL28A1, COL6A3, and CST3; COL28A1, COL6A3, and TNFRSF1A; COL28A1, CST3, and TNFRSF1A; UBE2G2, REG1A, and REG1B; UBE2G2, REG1A, and COL6A3; UBE2G2, REG1A, and CST3; UBE2G2, REG1A, and TNFRSF1A; UBE2G2, REG1B, and COL6A3; UBE2G2, REG1B, and CST3; UBE2G2, REG1B, and TNFRSF1A; UBE2G2, COL6A3, and CST3; UBE2G2, COL6A3, and TNFRSF1A; UBE2G2, CST3, and TNFRSF1A; REG1A, REG1B, and COL6A3; REG1A, REG1B, and CST3; REG1A, REG1B, and TNFRSF1A; REG1A, COL6A3, and CST3; REG1A, COL6A3, and TNFRSF1A; REG1A, CST3, and TNFRSF1A; REG1B, COL6A3, and CST3; REG1B, COL6A3, and TNFRSF1A; REG1B, CST3, and TNFRSF1A; or COL6A3, CST3, and TNFRSF1A.

74. The method of any one of claims 70 to 73, further comprising measuring one or more of COL28A1, UBE2G2, and REG1B.

75. A method comprising:
a) contacting a sample from a human subject with a set of capture reagents, wherein each capture reagent has affinity for a different protein of the set of proteins comprising three, four, five, six, seven, eight, nine or ten proteins selected from the group consisting of HAVCR1, FSTL3, RGMB, COL28A1, UBE2G2, REG1A, REG1B, COL6A3, CST3, and TNFRSF1A in a sample from a subject; and
b) measuring the level of each protein of the set of proteins with the set of capture reagents.

76. The method of claim 75, wherein the set of capture reagents are selected from aptamers, antibodies and a combinations of aptamers and antibodies.

77. The method of claim 75, wherein the sample is selected from blood, plasma, serum or urine.

78. The method of any one of claims 75 to 77, wherein the method comprises measuring HAVCR1, FSTL3, and RGMB; HAVCR1, FSTL3, and COL28A1; HAVCR1, FSTL3, and UBE2G2; HAVCR1, FSTL3, and REG1A; HAVCR1, FSTL3, and REG1B; HAVCR1, FSTL3, and COL6A3; HAVCR1, FSTL3, and CST3; HAVCR1, FSTL3, and TNFRSF1; HAVCR1, RGMB, and COL28A1; HAVCR1, RGMB, and UBE2G2; HAVCR1, RGMB, and REG1A; HAVCR1, RGMB, and REG1B; HAVCR1, RGMB, and COL6A3; HAVCR1, RGMB, and CST3; HAVCR1, RGMB, and TNFRSF1A; HAVCR1, COL28A1, and UBE2G2; HAVCR1, COL28A1, and REG1A; HAVCR1, COL28A1, and REG1B; HAVCR1, COL28A1, and COL6A3; HAVCR1, COL28A1, and CST3; HAVCR1, COL28A1, and TNFRSF1A; HAVCR1, UBE2G2, and REG1A; HAVCR1, UBE2G2, and REG1B; HAVCR1, UBE2G2, and COL6A3; HAVCR1, UBE2G2, and CST3; HAVCR1, UBE2G2, and TNFRSF1A; HAVCR1, REG1A, and REG1B; HAVCR1, REG1A, and COL6A3; HAVCR1, REG1A, and CST3; HAVCR1, REG1A, and TNFRSF1A; HAVCR1, REG1B, and COL6A3; HAVCR1, REG1B, and CST3; HAVCR1, REG1B, and TNFRSF1A; HAVCR1, COL6A3, and CST3; HAVCR1, COL6A3, and TNFRSF1A; HAVCR1, CST3, and TNFRSF1A; FSTL3, RGMB, and COL28A1; FSTL3, RGMB, and UBE2G2; FSTL3, RGMB, and REG1A; FSTL3, RGMB, and REG1B; FSTL3, RGMB, and COL6A3; FSTL3, RGMB, and CST3; FSTL3, RGMB, and TNFRSF1A; FSTL3, COL28A1, and UBE2G2; FSTL3, COL28A1, and REG1A; FSTL3, COL28A1, and REG1B; FSTL3, COL28A1, and COL6A3; FSTL3, COL28A1, and CST3; FSTL3, COL28A1, and TNFRSF1A; FSTL3, UBE2G2, and REG1A; FSTL3, UBE2G2, and REG1B; FSTL3, UBE2G2, and COL6A3; FSTL3, UBE2G2, and CST3; FSTL3, UBE2G2, and TNFRSF1A; FSTL3, REG1A, and REG1B; FSTL3, REG1A, and COL6A3; FSTL3, REG1A, and CST3; FSTL3, REG1A, and TNFRSF1A; FSTL3, REG1B, and COL6A3; FSTL3, REG1B, and CST3; FSTL3, REG1B, and TNFRSF1A; FSTL3, COL6A3, and CST3; FSTL3, COL6A3, and TNFRSF1A; FSTL3, CST3, and TNFRSF1A; RGMB, COL28A1, and UBE2G2; RGMB, COL28A1, and REG1A; RGMB, COL28A1, and REG1B; RGMB, COL28A1, and COL6A3; RGMB, COL28A1, and CST3; RGMB, COL28A1, and TNFRSF1A; RGMB, UBE2G2, and REG1A; RGMB, UBE2G2, and REG1B; RGMB, UBE2G2, and COL6A3; RGMB, UBE2G2, and CST3; RGMB, UBE2G2, and TNFRSF1A; RGMB, REG1A, and REG1B; RGMB, REG1A, and COL6A3; RGMB, REG1A, and CST3; RGMB, REG1A, and TNFRSF1A; RGMB, REG1B, and COL6A3; RGMB, REG1B, and CST3; RGMB, REG1B, and TNFRSF1A; RGMB, COL6A3, and CST3; RGMB, COL6A3, and TNFRSF1A; RGMB, CST3, and TNFRSF1A; COL28A1, UBE2G2, and REG1A; COL28A1, UBE2G2, and REG1B; COL28A1, UBE2G2, and COL6A3; COL28A1, UBE2G2, and CST3; COL28A1, UBE2G2, and TNFRSF1A; COL28A1, REG1A, and REG1B; COL28A1, REG1A, and COL6A3; COL28A1, REG1A, and CST3; COL28A1, REG1A, and TNFRSF1A; COL28A1, REG1B, and COL6A3; COL28A1, REG1B, and CST3; COL28A1, REG1B, and TNFRSF1A; COL28A1, COL6A3, and CST3; COL28A1, COL6A3, and TNFRSF1A; COL28A1, CST3, and TNFRSF1A; UBE2G2, REG1A, and REG1B; UBE2G2, REG1A, and COL6A3; UBE2G2, REG1A, and CST3; UBE2G2, REG1A, and TNFRSF1A; UBE2G2, REG1B, and COL6A3; UBE2G2, REG1B, and CST3; UBE2G2, REG1B, and TNFRSF1A; UBE2G2, COL6A3, and CST3; UBE2G2, COL6A3, and TNFRSF1A; UBE2G2, CST3, and TNFRSF1A; REG1A, REG1B, and COL6A3; REG1A, REG1B, and CST3; REG1A, REG1B, and TNFRSF1A; REG1A, COL6A3, and CST3; REG1A, COL6A3, and TNFRSF1A; REG1A, CST3, and TNFRSF1A; REG1B, COL6A3, and CST3; REG1B, COL6A3, and TNFRSF1A; REG1B, CST3, and TNFRSF1A; or COL6A3, CST3, and TNFRSF1A.

79. The method of any one of claims 75 to 78, further comprising measuring one or more of COL28A1, UBE2G2, and REG1B.

80. A method comprising:
a) measuring the level of HAVCR1 protein, and the level of at least one, two, three, four, five, six, seven, eight, or nine proteins selected from the group consisting of FSTL3, RGMB, COL28A1, UBE2G2, REG1A, REG1B, COL6A3, CST3, and TNFRSF1A in a sample from a human subject; and
b) identifying the human subject as being at relative risk for developing progressive chronic renal insufficiency within a 4 year period based on the level of HAVCR1 and the level of the at least one, two, three, four, five, six, seven, eight, or nine proteins.

81. A method comprising:
a) measuring the level of FSTL3 protein, and the level of at least one, two, three, four, five, six, seven, eight, or nine proteins selected from the group consisting of HAVCR1, RGMB, COL28A1, UBE2G2, REG1A, REG1B, COL6A3, CST3, and TNFRSF1A in a sample from a human subject; and
b) identifying the human subject as being at relative risk for developing progressive chronic renal insufficiency within a 4 year period based on the level of FSTL3 and the level of the at least one, two, three, four, five, six, seven, eight, or nine proteins.

82. A method comprising:
a) measuring the level of RGMB protein, and the level of at least one, two, three, four, five, six, seven, eight, or nine proteins selected from the group consisting of HAVCR1, FSTL3, COL28A1, UBE2G2, REG1A, REG1B, COL6A3, CST3, and TNFRSF1A in a sample from a human subject; and
b) identifying the human subject as being at relative risk for developing progressive chronic renal insufficiency within a 4 year period based on the level of RGMB and the level of the at least one, two, three, four, five, six, seven, eight, or nine proteins.

83. A method comprising:
a) measuring the level of REG1A protein, and the level of at least one, two, three, four, five, six, seven, eight, or nine proteins selected from the group consisting of HAVCR1, FSTL3, RGMB, COL28A1, UBE2G2, REG1B, COL6A3, CST3, and TNFRSF1A in a sample from a human subject; and
b) identifying the human subject as being at relative risk for developing progressive chronic renal insufficiency within a 4 year period based on the level of REG1A and the level of the at least one, two, three, four, five, six, seven, eight, or nine proteins.

84. A method comprising:
a) measuring the level of COL6A3 protein, and the level of at least one, two, three, four, five, six, seven, eight, or nine proteins selected from the group consisting of HAVCR1, FSTL3, RGMB, COL28A1, UBE2G2, REG1A, REG1B, CST3, and TNFRSF1A in a sample from a human subject; and
b) identifying the human subject as being at relative risk for developing progressive chronic renal insufficiency within a 4 year period based on the level of COL6A3 and the level of the at least one, two, three, four, five, six, seven, eight, or nine proteins.

85. A method comprising:
a) measuring the level of CST3 protein, and the level of at least one, two, three, four, five, six, seven, eight, or nine proteins selected from the group consisting of HAVCR1, FSTL3, RGMB, COL28A1, UBE2G2, REG1A, REG1B, COL6A3, and TNFRSF1A in a sample from a human subject; and
b) identifying the human subject as being at relative risk for developing progressive chronic renal insufficiency within a 4 year period based on the level of CST3 and the level of the at least one, two, three, four, five, six, seven, eight, or nine proteins.

86. A method comprising:
a) measuring the level of TNFRSF1A protein, and the level of at least one, two, three, four, five, six, seven, eight, or nine proteins selected from the group consisting of HAVCR1, FSTL3, RGMB, COL28A1, UBE2G2, REG1A, REG1B, COL6A3, and CST3 in a sample from a human subject; and
b) identifying the human subject as being at relative risk for developing progressive chronic renal insufficiency within a 4 year period based on the level of TNFRSF1A and the level of the at least one, two, three, four, five, six, seven, eight, or nine proteins.

87. The method of any one of claims 80 to 86, wherein the measuring is performed using mass spectrometry, an aptamer based assay and/or an antibody based assay.

88. The method of any one of claims 80 to 87, wherein the sample is selected from blood, plasma, serum or urine.

89. A method comprising:
a) contacting a sample from a human subject with a set of capture reagents, wherein each capture reagent has affinity for a different protein of the set of proteins comprising HAVCR1 protein, and at least one, two, three, four, five, six, seven, eight, or nine proteins selected from the group consisting of FSTL3, RGMB, COL28A1, UBE2G2, REG1A, REG1B, COL6A3, CST3, and TNFRSF1A; and
b) measuring the level of each protein of the set of proteins with the set of capture reagents.

90. A method comprising:
a) contacting a sample from a human subject with a set of capture reagents, wherein each capture reagent has affinity for a different protein of the set of proteins comprising FSTL3 protein, and at least one, two, three, four, five, six, seven, eight, or nine proteins selected from the group consisting of HAVCR1, RGMB, COL28A1, UBE2G2, REG1A, REG1B, COL6A3, CST3, and TNFRSF1A; and
b) measuring the level of each protein of the set of proteins with the set of capture reagents.

91. A method comprising:
a) contacting a sample from a human subject with a set of capture reagents, wherein each capture reagent has affinity for a different protein of the set of proteins comprising RGMB protein, and at least one, two, three, four, five, six, seven, eight, or nine proteins selected from the group consisting of HAVCR1, FSTL3, COL28A1, UBE2G2, REG1A, REG1B, COL6A3, CST3, and TNFRSF1A; and
b) measuring the level of each protein of the set of proteins with the set of capture reagents.

92. A method comprising:
a) contacting a sample from a human subject with a set of capture reagents, wherein each capture reagent has affinity for a different protein of the set of proteins comprising REG1A protein, and at least one, two, three, four, five, six, seven, eight, or nine proteins selected from the group consisting of HAVCR1, FSTL3, RGMB, COL28A1, UBE2G2, REG1B, COL6A3, CST3, and TNFRSF1A; and
b) measuring the level of each protein of the set of proteins with the set of capture reagents.

93. A method comprising:
a) contacting a sample from a human subject with a set of capture reagents, wherein each capture reagent has affinity for a different protein of the set of proteins comprising COL6A3 protein, and at least one, two, three, four, five, six, seven, eight, or nine proteins selected from the group consisting of HAVCR1, FSTL3, RGMB, COL28A1, UBE2G2, REG1A, REG1B, CST3, and TNFRSF1A; and
b) measuring the level of each protein of the set of proteins with the set of capture reagents.

94. A method comprising:
a) contacting a sample from a human subject with a set of capture reagents, wherein each capture reagent has affinity for a different protein of the set of proteins comprising CST3 protein, and at least one, two, three, four, five, six, seven, eight, or nine proteins selected from the group consisting of HAVCR1, FSTL3, RGMB, COL28A1, UBE2G2, REG1A, REG1B, COL6A3, and TNFRSF1A; and
b) measuring the level of each protein of the set of proteins with the set of capture reagents.

95. A method comprising:
a) contacting a sample from a human subject with a set of capture reagents, wherein each capture reagent has affinity for a different protein of the set of proteins comprising TNFRSF1A protein, and at least one, two, three, four, five, six, seven, eight, or nine proteins selected from the group consisting of HAVCR1, FSTL3, RGMB, COL28A1, UBE2G2, REG1A, REG1B, COL6A3, and CST3; and
b) measuring the level of each protein of the set of proteins with the set of capture reagents

96. The method of any one of claims 89 to 95, wherein the set of capture reagents is selected from aptamers, antibodies and a combinations of aptamers and antibodies.

97. The method of any one of claims 89 to 96, wherein the sample is selected from blood, plasma, serum or urine.

98. The method of any one of claims 16 to 27, 32 to 41, 53 to 60, 75 to 79, and 89 to 97, further comprising identifying the human subject as being at relative risk for developing progressive chronic renal insufficiency within a 4 year period based on the level of each protein measured.

99. The method of any one of claims 1 to 15, 28 to 31, 42 to 52, 61 to 74, 80 to 88, and 98, wherein the relative risk for developing progressive chronic renal insufficiency within a 4 year period is based on input of the level of each protein measured in a statistical model.

100. The method of claim 99, wherein the model is a linear regression model.

101. The method of claim 99 or 100, wherein the model has an area under the curve (AUC) selected from 0.65, 0.7, 0.75, 0.77, or greater.

102. The method of any one of claims 99 to 101, where the model provides a binary prediction and/or a relative risk prediction for developing progressive chronic renal insufficiency within a 4 year period.

103. The method of any one of claims 99 to 102, wherein the model is based on the level of each of the proteins selected from HAVCR1, FSTL3, RGMB, COL28A1, UBE2G2, REG1A, REG1B, COL6A3, CST3, and TNFRSF1A.

104. The method of claim 103, wherein the model provides a binary prediction with a probability cut point X, wherein X<0.3, 0.31, 0.32, 0.32, 0.33, 0.34, 0.35, or 0.3533 predicts no risk for developing progressive chronic renal insufficiency within a 4 year period and X≥0.3, 0.31, 0.32, 0.33, 0.34, 0.35, or 0.3533 predicts risk for developing progressive chronic renal insufficiency within a 4 year period.

105. The method of claim 104, wherein the risk of developing progressive chronic renal insufficiency indicates a risk of an event selected from a 50% decline in estimated glomerular filtration rate (eGFR); a diagnosis that kidney dialysis is needed; a development of eGFR<15 ml/min/1.73 m2; a development of end stage renal disease (ESRD); and a diagnosis that a kidney transplantation is needed.

106. The method of claim 103, wherein the model provides the relative risk prediction for developing progressive chronic renal insufficiency within a 4 year period based on the level of each of the proteins selected from HAVCR1, FSTL3, RGMB, COL28A1, UBE2G2, REG1A, REG1B, COL6A3, CST3, and TNFRSF1A.

107. The method of claim 98, wherein the level of each protein measured is determined from a relative florescence unit (RFU) or a protein concentration.

108. The method of claim 103, wherein the model provides for a relative risk for developing progressive chronic renal insufficiency within a 4 year period.

109. The method of claim 103, wherein the relative risk is selective from mild and severe.

110. The method of claim 103, wherein the relative risk is a probability calculation.

111. The method of claim 103, wherein the relative risk is a value range used to predict the development of progressive chronic renal insufficiency within a 4 year period.
